(19) United States
(12) Patent Application Publication
(10) Pub. No.: US 2024/0254135 A1
(43) Pub. Date: Aug. 1, 2024

(54) POLYPHENOL COMPOUNDS

(71) Applicant: Epirium Bio Inc., San Diego, CA (US)

(72) Inventors: Sundeep DUGAR, San Jose, CA (US); Bhavesh DAVE, Maharashtra (IN); Sohail AHMAD, Uttar Pradesh (IN); Somdutta SEN, Manesar (IN)

(21) Appl. No.: 18/391,125

(22) Filed: Dec. 20, 2023

Related U.S. Application Data
(60) Provisional application No. 63/434,416, filed on Dec. 21, 2022.

(51) Int. Cl. C07D 491/052; C07D 215/20; C07D 311/62; C07D 405/04

(52) U.S. Cl. CPC C07D 491/052; C07D 215/20; C07D 311/62; C07D 405/04

(57) ABSTRACT

The present application discloses derivatives of polyphenols, pharmaceutically acceptable salts, stereoisomers or tautomers thereof, and processes for preparation thereof. The compounds and compositions described herein can be used in therapy.

### FIELD

The present disclosure relates generally to polyphenols and related compounds. Process for preparation of the compounds, compositions comprising the compounds and methods of use are also provided.

### BACKGROUND

Polyphenolic natural products are of current interest because of their various biological activities, their occurrence in foodstuffs, and hence their relevance for human health. Polyphenolic natural products have two or more hydroxyl groups on their aromatic rings.

Representative examples include: (−)-epiafzelechin, (+)-catechin, (−)-epicatechin, (−)-gallocatechin, (−)-epigallocatechin, their respective 3-gallate esters, as well as two 3-(30-methyl)-gallate esters, herein referred to collectively as “catechins”. (+)-Catechin, (−)-catechins, (+)-epicatechin and (−)-epicatechin are flavon-3-ols. These flavonols are present in the human diet in chocolate, fruits, vegetables and wine and have found use in the treatment of acute coronary syndromes, including but not limited to myocardial infarction and angina; acute ischemic events in other organs and tissues, renal injury, renal ischemia and diseases of the aorta and its branches; injuries arising from medical interventions, including but not limited to coronary artery bypass grafting (CABG) procedures and aneurysm repair; cancer; and metabolic diseases, diabetes mellitus and other such disorders.

Though such polyphenols are used widely, in many instances they have drawbacks such as low potency, and undesirable pharmacodynamic or pharmacokinetic profiles. Hence there is a need to develop new polyphenol compounds.

### SUMMARY

Provided herein are compounds, salts thereof, pharmaceutical compositions of the foregoing and methods of making and using the same. In one aspect, provided is a compound of formula (I):

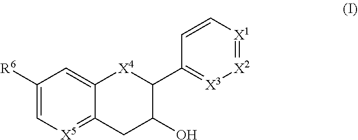

or a pharmaceutically acceptable salt thereof, wherein X¹, X², X³, X⁴, R⁴, X⁵, and R⁶ are as detailed herein.

In one aspect, provided is a compound of formula (II):

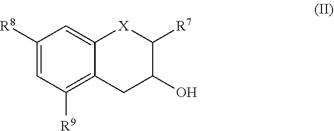

or a pharmaceutically acceptable salt thereof, wherein X, R⁷, R⁸, and R⁹ are as detailed herein.

In one aspect, provided is a compound of formula (III):

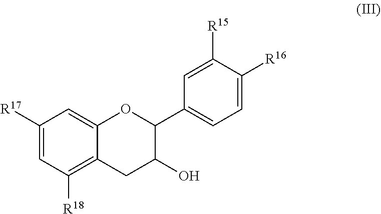

or a pharmaceutically acceptable salt thereof, wherein R¹⁵, R¹⁶, R¹⁷, and R¹⁸ are as detailed herein.

Also provided is a pharmaceutical composition comprising a compound of any formula herein, including formulas (I), (II), or (III), a pharmaceutically acceptable salt thereof, and a pharmaceutically acceptable carrier.

In yet another aspect, the compounds and the pharmaceutical compositions provided herein are useful in treating diseases or disorders that would benefit from modification of Electron transfer Chain (ETC) particularly electron transfer chain IV. Provided is a method of preventing and/or treating such a disease or disorder in a subject in need thereof comprising administering to the subject a therapeutically effective amount of a compound as detailed herein, including but not limited to a compound of formulas (I), (II), or (III), a pharmaceutically acceptable salt thereof, or a pharmaceutical composition comprising such compound or salt. In some embodiments, provided are methods for prophylactic and/or therapeutic treatment of conditions related to mitochondrial dysfunction.

### BRIEF DESCRIPTION OF DRAWINGS

FIG. 1. ATP synthesis and hydrolysis of compounds 77b, 75b, and (+)-epicatechin in fresh heart mitochondria.

FIG. 2. Oligomycin controls of ATP synthesis and hydrolysis in fresh heart mitochondria.

FIG. 3. ATP hydrolysis assay of 77b, 75b, and (+)-epicatechin in frozen heart mitochondria (repetition #1).

FIG. 4. ATP hydrolysis assay of 77b, 75b, and (+)-epicatechin in frozen heart mitochondria (repetition #2).

### DETAILED DESCRIPTION

The present application discloses derivatives of polyphenols, pharmaceutically acceptable salts, stereoisomers or tautomers thereof, and processes for preparation thereof. The compounds and compositions described herein can be used in therapy.

### Definitions

As used herein and in the appended claims, the singular forms “a”, “an” and “the” include plural forms, unless the context clearly dictates otherwise.

As used herein, and unless otherwise specified, the terms “about” and “approximately,” when used in connection with doses, amounts, molar percent, or weight percent of ingredients of a composition or a dosage form, mean a dose, amount, molar percent, or weight percent that is recognized by those of ordinary skill in the art to provide a pharmacological effect equivalent to that obtained from the specified dose, amount, molar percent, or weight percent. Specifically, the terms “about” and “approximately,” when used in this context, contemplate a dose, amount, molar percent, or weight percent within 15%, within 10%, within 5%, within 4%, within 3%, within 2%, within 1%, or within 0.5% of the specified dose, amount, molar percent, or weight percent.

“Alkyl” as used herein refers to and includes, unless otherwise stated, a saturated linear (i.e., unbranched) or branched univalent hydrocarbon chain or combination thereof, having the number of carbon atoms designated (i.e., C₁-C₁₀ means one to ten carbon atoms). Particular alkyl groups are those having 1 to 20 carbon atoms (a “C₁-C₂₀ alkyl”), having 1 to 10 carbon atoms (a “C₁-C₁₀ alkyl”), having 6 to 10 carbon atoms (a “C₆-C₁₀ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

“Cycloalkyl” as used herein refers to and includes, unless otherwise stated, saturated cyclic univalent hydrocarbon structures, having the number of carbon atoms designated (i.e., C₃-C₁₀ means three to ten carbon atoms). Cycloalkyl can consist of one ring, such as cyclohexyl, or multiple rings, such as adamantyl. A cycloalkyl comprising more than one ring may be fused, spiro or bridged, or combinations thereof. Particular cycloalkyl groups are those having from 3 to 12 annular carbon atoms. A preferred cycloalkyl is a cyclic hydrocarbon having from 3 to 8 annular carbon atoms (a “C₃-C₈ cycloalkyl”), having 3 to 6 carbon atoms (a “C₃-C₆ cycloalkyl”), or having from 3 to 4 annular carbon atoms (a “C₃-C₄ cycloalkyl”). Examples of cycloalkyl include, but are not limited to, cyclopropyl, cyclobutyl, cyclopentyl, cyclohexyl, cycloheptyl, norbornyl, and the like.

“Aryl” or “Ar” as used herein refers to an unsaturated aromatic carbocyclic group having a single ring (e.g., phenyl) or multiple condensed rings (e.g., naphthyl or anthryl) which condensed rings may or may not be aromatic. Particular aryl groups are those having from 6 to 14 annular carbon atoms (a “C₆-C₁₄ aryl”). An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position.

“Heteroaryl” as used herein refers to an unsaturated aromatic cyclic group having from 1 to 14 annular carbon atoms and at least one annular heteroatom, including but not limited to heteroatoms such as nitrogen, oxygen and sulfur. A heteroaryl group may have a single ring (e.g., pyridyl, furyl) or multiple condensed rings (e.g., indolizinyl, benzothienyl) which condensed rings may or may not be aromatic. Particular heteroaryl groups are 5 to 14-membered rings having 1 to 12 annular carbon atoms and 1 to 6 annular heteroatoms independently selected from nitrogen, oxygen and sulfur, 5 to 10-membered rings having 1 to 8 annular carbon atoms and 1 to 4 annular heteroatoms independently selected from nitrogen, oxygen and sulfur, or 5, 6 or 7-membered rings having 1 to 5 annular carbon atoms and 1 to 4 annular heteroatoms independently selected from nitrogen, oxygen and sulfur. In one variation, particular heteroaryl groups are monocyclic aromatic 5-, 6- or 7-membered rings having from 1 to 6 annular carbon atoms and 1 to 4 annular heteroatoms independently selected from nitrogen, oxygen and sulfur. In another variation, particular heteroaryl groups are polycyclic aromatic rings having from 1 to 12 annular carbon atoms and 1 to 6 annular heteroatoms independently selected from nitrogen, oxygen and sulfur. A heteroaryl group having more than one ring where at least one ring is non-aromatic may be connected to the parent structure at either an aromatic ring position or at a non-aromatic ring position. In one variation, a heteroaryl group having more than one ring where at least one ring is non-aromatic is connected to the parent structure at an aromatic ring position. A heteroaryl group may be connected to the parent structure at a ring carbon atom or a ring heteroatom.

“Heterocycle”, “heterocyclic”, or “heterocyclyl” as used herein refers to a saturated or an unsaturated non-aromatic cyclic group having a single ring or multiple condensed rings, and having from 1 to 14 annular carbon atoms and from 1 to 6 annular heteroatoms, such as nitrogen, sulfur or oxygen, and the like. A heterocycle comprising more than one ring may be fused, bridged or spiro, or any combination thereof, but excludes heteroaryl groups. The heterocyclyl group may be optionally substituted independently with one or more substituents described herein. Particular heterocyclyl groups are 3 to 14-membered rings having 1 to 13 annular carbon atoms and 1 to 6 annular heteroatoms independently selected from nitrogen, oxygen and sulfur, 3 to 12-membered rings having 1 to 11 annular carbon atoms and 1 to 6 annular heteroatoms independently selected from nitrogen, oxygen and sulfur, 3 to 10-membered rings having 1 to 9 annular carbon atoms and 1 to 4 annular heteroatoms independently selected from nitrogen, oxygen and sulfur, 3 to 8-membered rings having 1 to 7 annular carbon atoms and 1 to 4 annular heteroatoms independently selected from nitrogen, oxygen and sulfur, or 3 to 6-membered rings having 1 to 5 annular carbon atoms and 1 to 4 annular heteroatoms independently selected from nitrogen, oxygen and sulfur. In one variation, heterocyclyl includes monocyclic 3-, 4-, 5-, 6- or 7-membered rings having from 1 to 2, 1 to 3, 1 to 4, 1 to 5, or 1 to 6 annular carbon atoms and 1 to 2, 1 to 3, or 1 to 4 annular heteroatoms independently selected from nitrogen, oxygen and sulfur. In another variation, heterocyclyl includes polycyclic non-aromatic rings having from 1 to 12 annular carbon atoms and 1 to 6 annular heteroatoms independently selected from nitrogen, oxygen and sulfur.

“Halo” or “halogen” refers to elements of the Group 17 series having atomic number 9 to 85. Preferred halo groups include the radicals of fluorine, chlorine, bromine and iodine. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl. An alkyl group in which each hydrogen is replaced with a halo group can be referred to as a “perhaloalkyl.” An exemplary perhaloalkyl group is trifluoromethyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

Asymmetric centers exist in the compounds disclosed herein. These centers are designated by the symbols “R” or “S,” depending on the configuration of substituents around the chiral carbon atom. It should be understood that stereochemical isomeric forms, including diastereomeric, enantiomeric, and epimeric forms as well as d-isomers and 1-isomers, and mixtures thereof are encompassed. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. All cis, trans, syn, anti, entgegen (E), and zusammen (Z) isomers as well as the appropriate mixtures thereof are included. Additionally, compounds may exist as tautomers; all tautomeric isomers are provided. Additionally, the compounds disclosed herein can exist in unsolvated as well as solvated forms with pharmaceutically acceptable solvents such as water, ethanol, and the like. In general, the solvated forms are considered equivalent to the unsolvated forms.

As used herein, “therapeutically effective amount” indicates an amount that results in a desired pharmacological and/or physiological effect for the condition. The effect may be prophylactic in terms of completely or partially preventing a condition or symptom thereof and/or may be therapeutic in terms of a partial or complete cure for the condition and/or adverse effect attributable to the condition.

As used herein, the term “pharmaceutically acceptable excipient,” and cognates thereof, refers to adjuvants, binders, diluents, etc. known to the skilled artisan that are suitable for administration to a subject (e.g., a mammal or non-mammal). Combinations of two or more excipients are also contemplated. The pharmaceutically acceptable excipient(s) and any additional components, as described herein, should be compatible for use in the intended route of administration (e.g., oral, parenteral) for a particular dosage form, as would be recognized by the skilled artisan.

“Pharmaceutically acceptable salts” are those salts which retain at least some of the biological activity of the free (non-salt) compound and which can be administered as drugs or pharmaceuticals to a subject. Such salts, for example, include, without limitation: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound in its free acid or base form with a suitable organic or inorganic base or acid, respectively, and isolating the salt thus formed during subsequent purification.

The terms “treat,” “treating,” and “treatment” are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. Often, the beneficial effects that a subject derives from a therapeutic agent do not result in a complete cure of the disease, disorder or condition.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

### Compounds

In some embodiments, provided is a compound of formula (I),

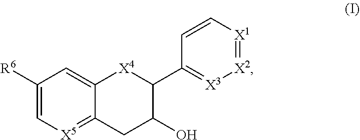

or a pharmaceutically acceptable salt, stereoisomer or tautomer thereof, wherein:
X¹ is N or CR¹;
R¹ is H, OH, halo, or C₁-6 alkoxy;
X² is N or CR²;
R² is H, OH, halo, C₁-6 alkyl optionally substituted with one or more halo, or C₁-6 alkoxy;
X³ is N or CR³;
R³ is H or OH;
X⁴ is O or NR⁴;
R⁴ is H or CH₃;
X⁵ is N or CR⁵;
R⁵ is H, OH, or C₁-6 alkoxy;
provided that if X⁴ is NR⁴, none of X¹, X², X³, and X⁵ is N, and if X⁴ is O, then one of X¹, X², X³, and X⁵ is N; and
R⁶ is H, OH, or C₁₋₆ alkoxy.

In some embodiments, X¹ is N. In some embodiments, X¹ is CR¹. In some embodiments, R¹ is H, OH, halo, or C₁-C₆ alkoxy. In some embodiments, R¹ is C₁-C₆ alkoxy. In some embodiments, R¹ is methoxy. In some embodiments, R¹ is halo. In some embodiments, R¹ is fluoro. In some embodiments, R¹ is H. In some embodiments, R¹ is OH.

In some embodiments, X² is N. In some embodiments, X² is CR². In some embodiments, R² is H, OH, halo, C₁-C₆ alkoxy, or C₁-C₆ alkyl, wherein C₁-C₆ alkyl is optionally substituted with halogen. In some embodiments, R² is H. In some embodiments, R² is OH. In some embodiments, R² is C₁-C₆ alkoxy. In some embodiments, R² is methoxy. In some embodiments, R² is C₁-C₆ alkyl. In some embodiments, R² is methyl. In some embodiments, R² is ethyl. In some embodiments, R² is methyl substituted with halo. In some embodiments, R² is CF₃. In some embodiments, R² is halo. In some embodiments, R² is fluoro.

In some embodiments, X³ is N. In some embodiments, X³ is CR³. In some embodiments, R³ is H. In some embodiments, R³ is OH.

In some embodiments, X⁴ is O. In some embodiments, X⁴ is NR⁴. In some embodiments, R⁴ is H. In some embodiments, R⁴ is C₁-C₆ alkyl. In some embodiments, R⁴ is methyl.

In some embodiments, X⁵ is N. In some embodiments, X⁵ is CR⁵. In some embodiments, R⁵ is H, OH, or C₁-C₆ alkoxy. In some embodiments, R⁵ is H. In some embodiments, R⁵ is OH. In some embodiments, R⁵ is C₁-C₆ alkoxy. In some embodiments, R⁵ is methoxy.

In some embodiments, X⁴ is NR⁴, and none of X¹, X², X³, and X⁵ is N. In some embodiments, X⁴ is O, and one of X¹, X², X³, and X⁵ is N.

In some embodiments, R⁶ is H, OH, or C₁₋₆ alkoxy. In some embodiments, R⁶ is H. In some embodiments, R⁶ is OH. In some embodiments, R⁶ is C₁₋₆ alkoxy. In some embodiments, R⁶ is methoxy.

The 6-membered ring comprising X¹, X², and X³ may contain zero to one nitrogen atoms. In some embodiments, the 6-membered ring comprising X¹, X², and X³ contains no nitrogen atoms. In some embodiments, the 6-membered ring comprising X¹, X², and X³ contains one nitrogen atom. In some embodiments, the 6-membered ring comprising X¹, X², and X³ is phenyl substituted with one or two substituents independently selected from the group consisting of halo, OH, C₁₋₆ alkyl substituted with halo, and C₁₋₆ alkoxy. In some embodiments, the 6-membered ring comprising X¹, X², and X³ is 2-pyridyl, 3-pyridyl or 4-pyridyl. In some embodiments, the 6-membered ring comprising X¹, X², and X³ is 2-pyridyl, 3-pyridyl or 4-pyridyl, each of which is substituted with one or two substituents independently selected from the group consisting of halo, OH, C₁₋₆ alkyl substituted with halo, and C₁₋₆ alkoxy. In some embodiments, the 6-membered ring comprising X¹, X², and X³ is selected from the group consisting of

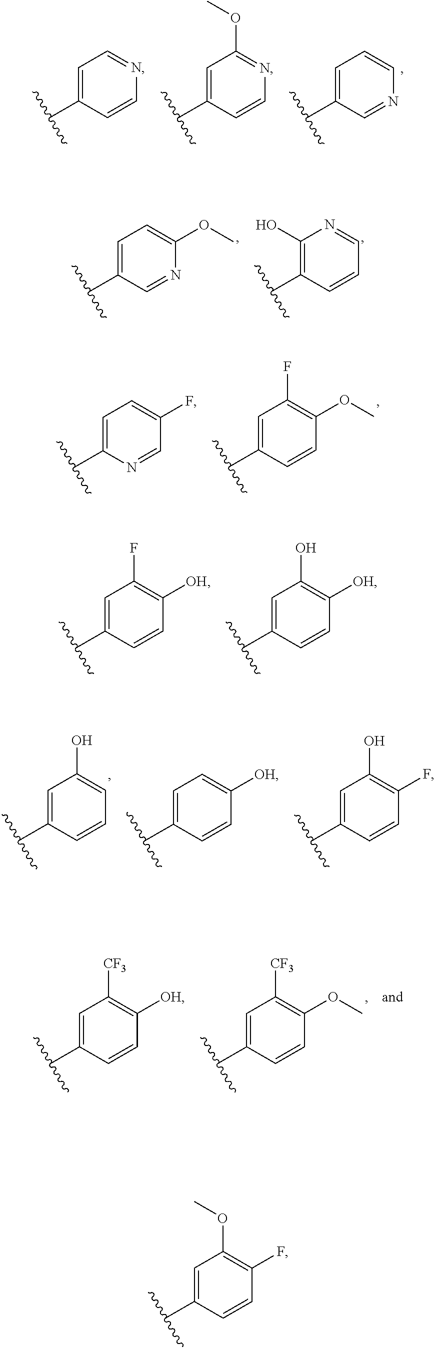

wherein the wavy line indicates attachment to the remainder of the Formula (I) structure.

In some embodiments, X¹ is N, and X² is C—H or C—OCH₃. In some embodiments, X² is N, and X³ is C—H or C—OH. In some embodiments, X³ is N, and X¹ is C—F. In some embodiments, X⁵ is N, X¹ is C—OH, and X² is C—H, C—OH or C—F. In some embodiments, X⁴ is N, X¹ is C—OH, and X² is C—F.

In some embodiments, provided is a compound of formula (I-1),

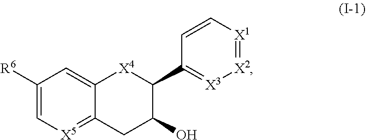

or a pharmaceutically acceptable salt, stereoisomer or tautomer thereof, wherein each variable is as defined for formula (I) or any variation thereof.

In some embodiments, provided is a compound of formula (I-2),

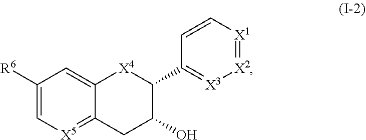

or a pharmaceutically acceptable salt, stereoisomer or tautomer thereof, wherein each variable is as defined for formula (I) or any variation thereof.

In some embodiments, provided is a compound of formula (I-3),

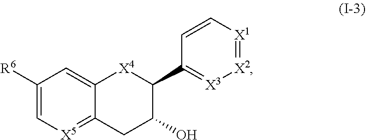

or a pharmaceutically acceptable salt, stereoisomer or tautomer thereof, wherein each variable is as defined for formula (I) or any variation thereof.

In some embodiments, provided is a compound of formula (I-4),

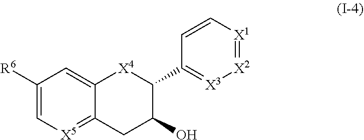

or a pharmaceutically acceptable salt, stereoisomer or tautomer thereof, wherein each variable is as defined for formula (I) or any variation thereof. In some embodiments, provided is a compound of formula (Ia),

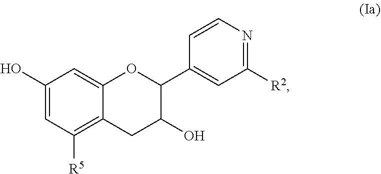

or a pharmaceutically acceptable salt, stereoisomer or tautomer thereof, wherein each variable is as defined for formula (I) or any variation thereof.

In some embodiments, provided is a compound of formula (Ib),

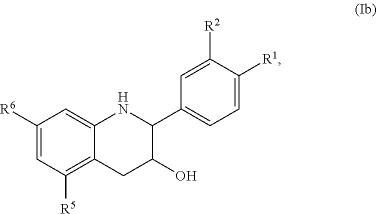

or a pharmaceutically acceptable salt, stereoisomer or tautomer thereof, wherein each variable is as defined for formula (I) or any variation thereof.

In some embodiments, provided is a compound of formula (Ic),

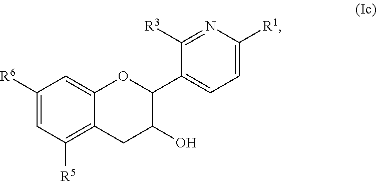

or a pharmaceutically acceptable salt, stereoisomer or tautomer thereof, wherein each variable is as defined for formula (I) or any variation thereof.

In some embodiments, provided is a compound of formula (Id),

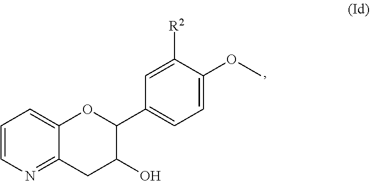

or a pharmaceutically acceptable salt, stereoisomer or tautomer thereof, wherein each variable is as defined for formula (I).

In some embodiments, provided is a compound of formula (Ie),

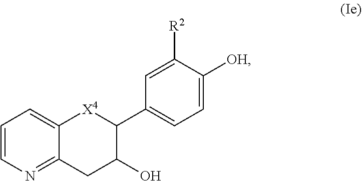

or a pharmaceutically acceptable salt, stereoisomer or tautomer thereof, wherein each variable is as defined for formula (I) or any variation thereof.

In some embodiments, provided is a compound of formula (If),

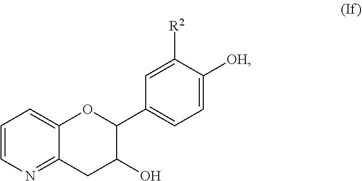

or a pharmaceutically acceptable salt, stereoisomer or tautomer thereof, wherein each variable is as defined for formula (I) or any variation thereof.

In some embodiments, provided is a compound of formula (Ig),

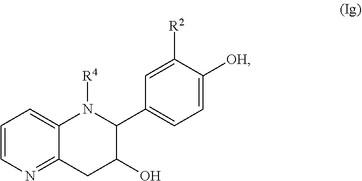

or a pharmaceutically acceptable salt, stereoisomer or tautomer thereof, wherein each variable is as defined for formula (I) or any variation thereof.

In some embodiments, provided is a compound of formula (Ih),

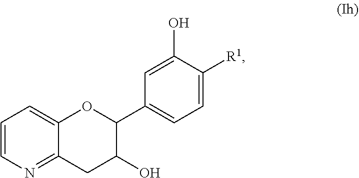

or a pharmaceutically acceptable salt, stereoisomer or tautomer thereof, wherein each variable is as defined for formula (I) or any variation thereof.

In some embodiments, provided is a compound of formula (Ii),

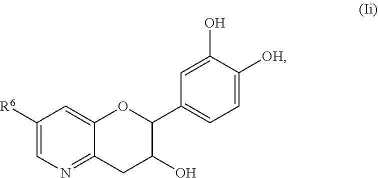

or a pharmaceutically acceptable salt, stereoisomer or tautomer thereof, wherein each variable is as defined for formula (I) or any variation thereof.

In some embodiments, the compound of Formula (I) is selected from the group consisting of:

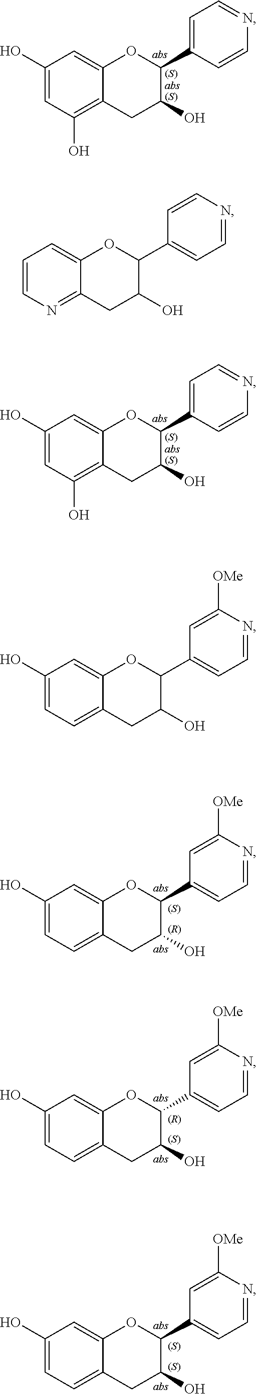

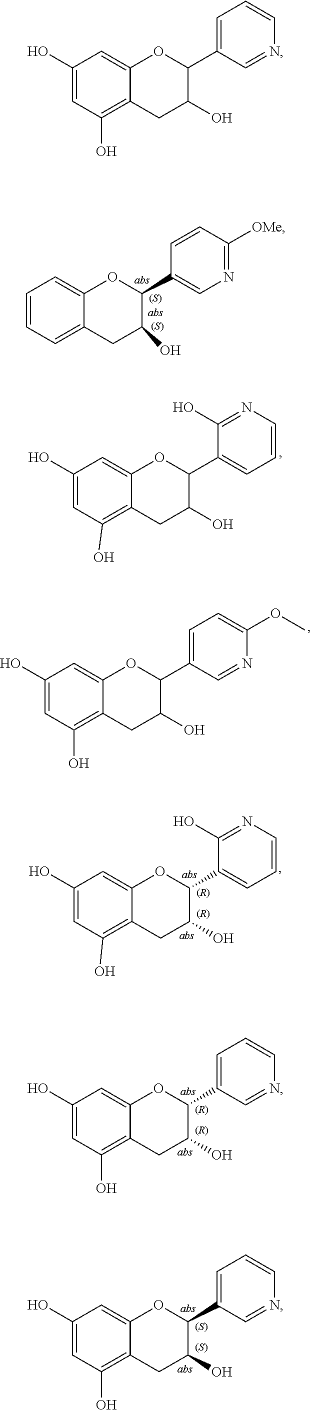

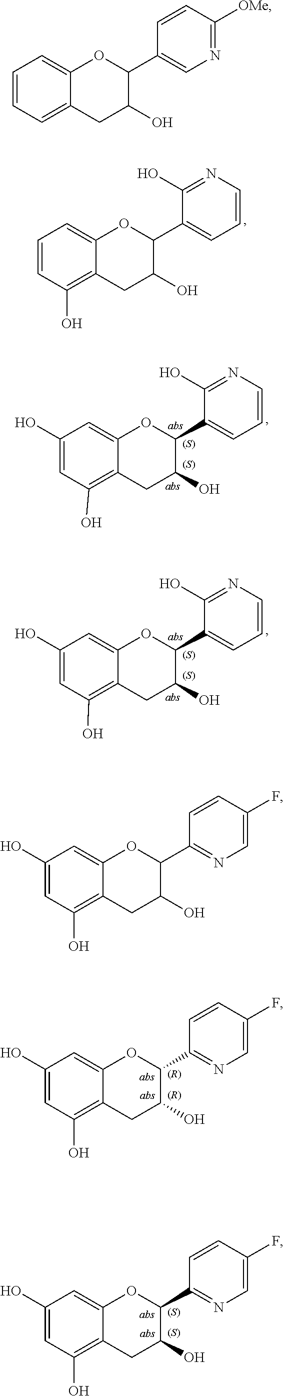

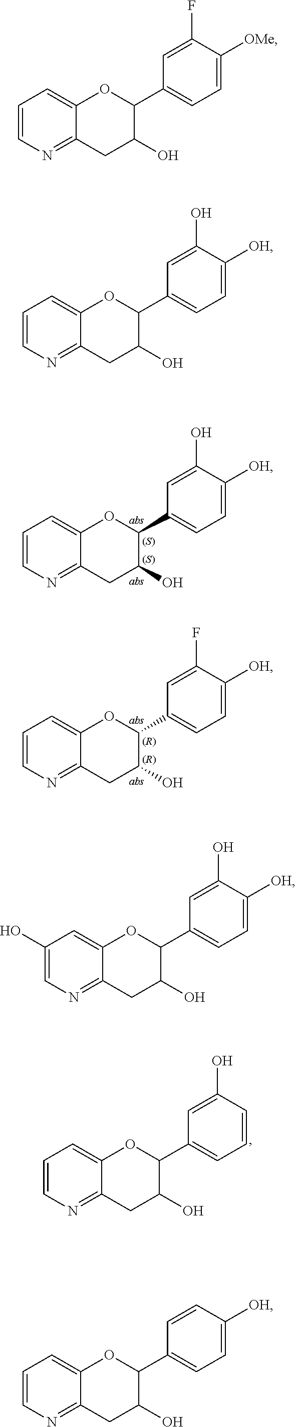

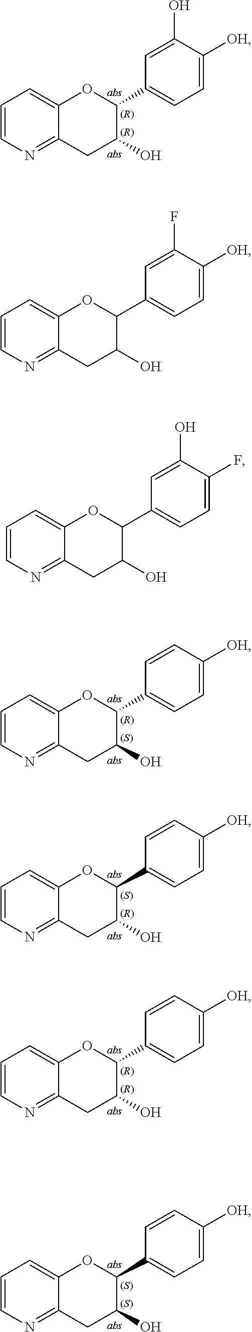

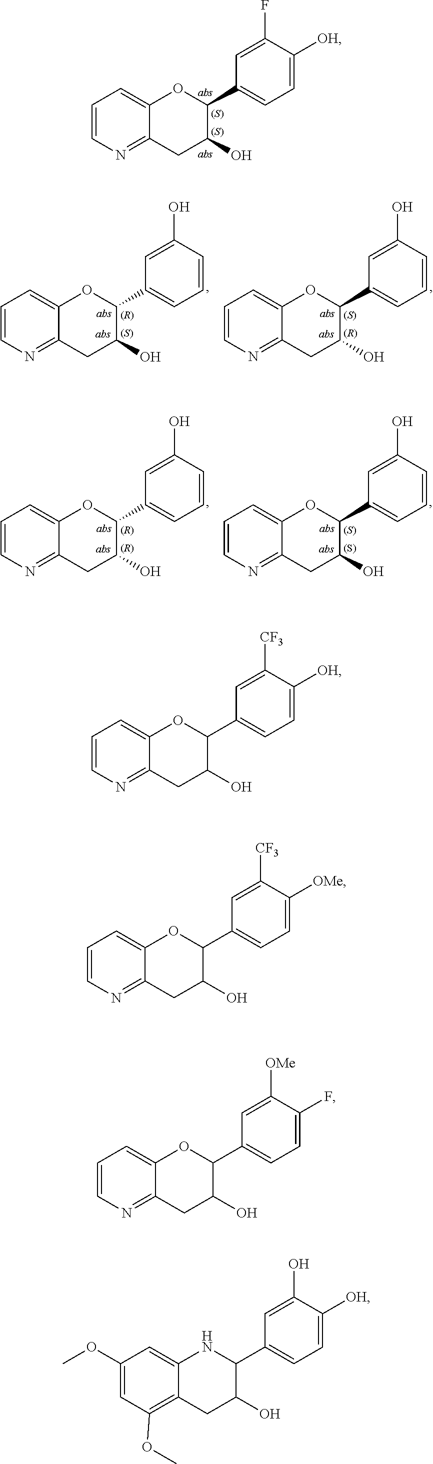

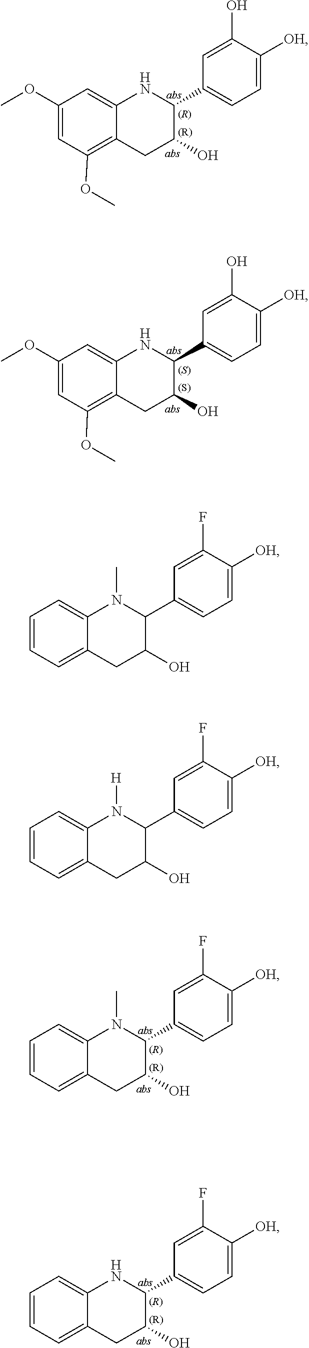

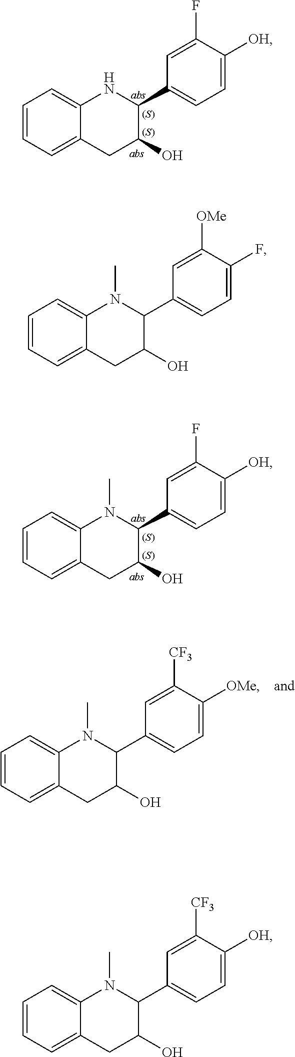

or a pharmaceutically acceptable salt, stereoisomer or tautomer thereof.

In some embodiments, provided is a compound of formula (II):

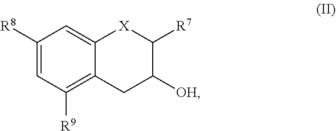

wherein:
X is O or NCH₃;
R⁷ is C₁₋₆ alkyl, C₃₋₁₀ cycloalkyl optionally substituted with one or more OH or C₁₋₆ alkoxy, or 5-6 membered heterocyclyl optionally substituted with one or more C₁₋₆ alkyl; and
R⁸ and R⁹ are each independently H or OH.

In some embodiments, X is O. In some embodiments, X is NCH₃.

In some embodiments, R⁷ is C₁₋₆ alkyl, C₃₋₁₀ cycloalkyl optionally substituted with one or more OH or C₁₋₆ alkoxy, or 5-6 membered heterocyclyl optionally substituted with one or more C₁₋₆ alkyl. In some embodiments, R⁷ is C₁₋₆ alkyl. In some embodiments, R⁷ is C₃₋₁₀ cycloalkyl. In some embodiments, R⁷ is cyclobutyl. In some embodiments, R⁷ is cyclohexyl. In some embodiments, R⁷ is cyclohexyl substituted with methoxy. In some embodiments, R⁷ is cyclohexyl substituted with OH. In some embodiments, R⁷ is piperidinyl. In some embodiments, R⁷ is piperidinyl substituted with methyl. In some embodiments, R⁷ is tetrahydropyranyl. In some embodiments, R⁷ is tetrahydrofuranyl.

In some embodiments, R⁸ is H. In some embodiments, R⁸ is OH. In some embodiments, R⁹ is H. In some embodiments, R⁹ is OH.

In some embodiments, R⁷ is tetrahydropyranyl, and R⁹ is OH. In some embodiments, R⁷ is cyclohexyl optionally substituted with OH or methoxy, and R⁹ is OH. In some embodiments, R⁷ is tetrahydropyranyl, and R⁹ is H.

In some embodiments, provided is a compound of formula (II-1):

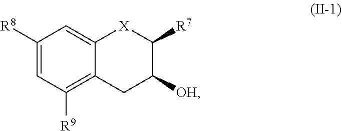

or a pharmaceutically acceptable salt, stereoisomer or tautomer thereof, wherein each variable is as defined for formula (II) or any variation thereof.

In some embodiments, provided is a compound of formula (II-2):

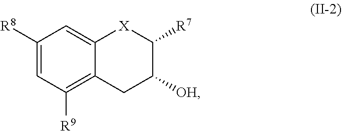

or a pharmaceutically acceptable salt, stereoisomer or tautomer thereof, wherein each variable is as defined for formula (II) or any variation thereof.

In some embodiments, provided is a compound of formula (II-3):

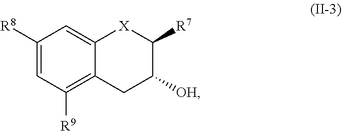

or a pharmaceutically acceptable salt, stereoisomer or tautomer thereof, wherein each variable is as defined for formula (II) or any variation thereof.

In some embodiments, provided is a compound of formula (II-4):

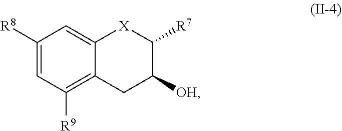

or a pharmaceutically acceptable salt, stereoisomer or tautomer thereof, wherein each variable is as defined for formula (II) or any variation thereof.

In some embodiments, provided is a compound of formula (IIa):

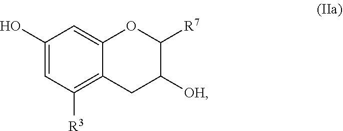

or a pharmaceutically acceptable salt, stereoisomer or tautomer thereof, wherein each variable is as defined in formula (II) or any variation thereof.

In some embodiments, provided is a compound of formula (IIb):

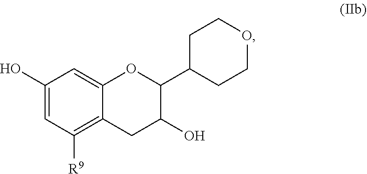

or a pharmaceutically acceptable salt, stereoisomer or tautomer thereof, wherein each variable is as defined in formula (II) or any variation thereof.

In some embodiments, provided is a compound of formula (IIc):

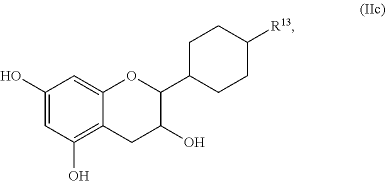

or a pharmaceutically acceptable salt, stereoisomer or tautomer thereof, wherein R¹³ is H, OH, or methoxy, and each of the remaining variables is as defined in formula (II) or any variation thereof.

In some embodiments, provided is a compound of formula (IId):

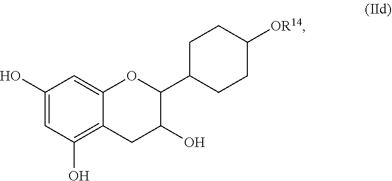

or a pharmaceutically acceptable salt, stereoisomer or tautomer thereof, wherein R¹⁴ is H or methyl, and each of the remaining variables is as defined in formula (II) or any variation thereof.

In some embodiments, the compound of formula (II) is selected from the group consisting of:

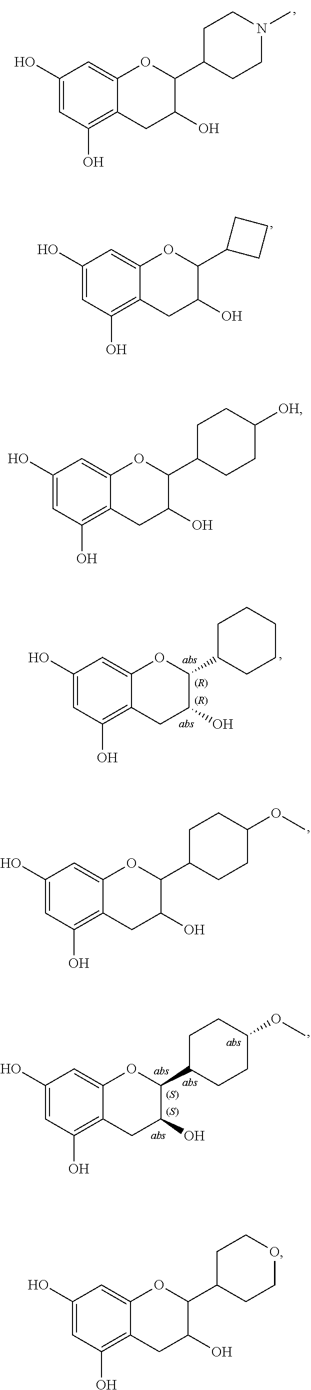

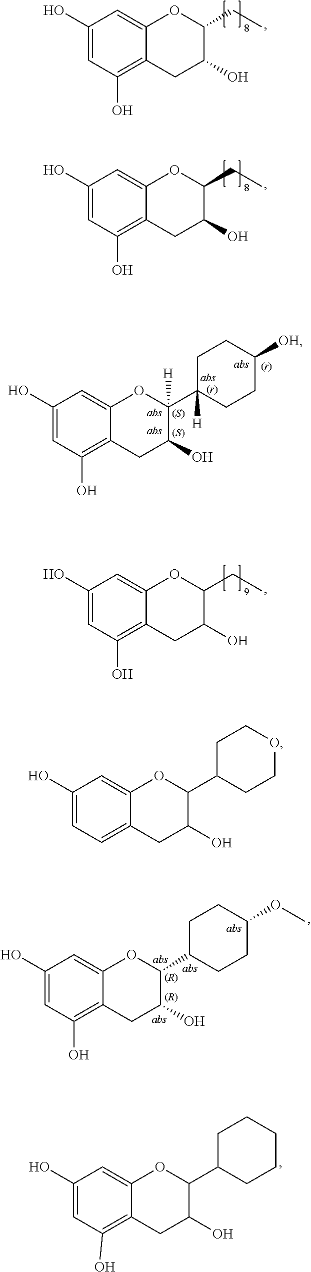

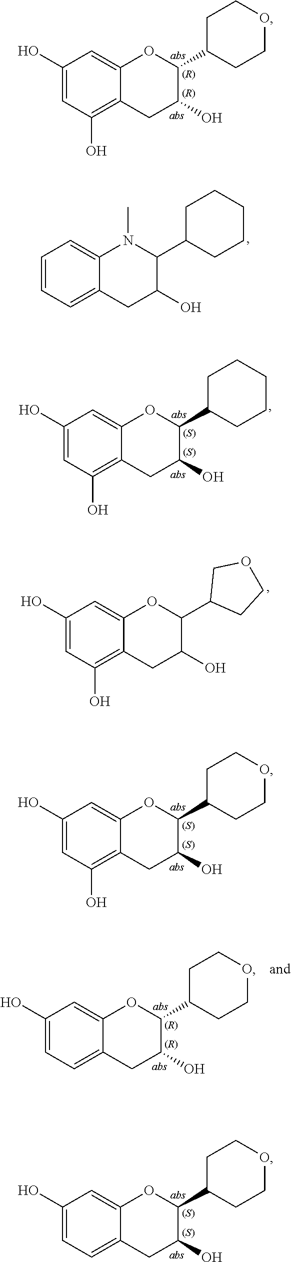

or a pharmaceutically acceptable salt, stereoisomer or tautomer thereof.

In some embodiments, provided is a compound of formula (III):

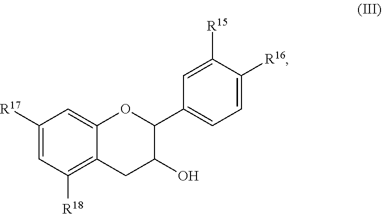

wherein
R¹⁵ is H, C₁₋₆ alkoxy, halo, or C₁₋₆ alkyl optionally substituted with halo;
R¹⁶ is H, halo, or OH; and
R¹⁷ and R¹⁸ are each independently H or OH.

In some embodiments, at least one of R¹⁵ and R¹⁶ is halo, or R¹⁵ is C₁₋₆ alkyl substituted with halo.

In some embodiments, R¹⁵ is H. In some embodiments, R¹⁵ is C₁₋₆ alkoxy. In some embodiments, R¹⁵ is methoxy. In some embodiments, R¹⁵ is halo. In some embodiments, R¹⁵ is fluoro. In some embodiments, R¹⁵ is C₁₋₆ alkyl. In some embodiments, R¹⁵ is C₁₋₆ alkyl optionally substituted with halo. In some embodiments, R¹⁵ is C₁₋₆ alkyl optionally substituted with fluoro.

In some embodiments, R¹⁶ is H. In some embodiments, R¹⁶ is halo. In some embodiments, R¹⁶ is fluoro. In some embodiments, R¹⁶ is OH.

In some embodiments, R¹⁷ is H. In some embodiments, R¹⁷ is OH. In some embodiments, R¹⁸ is H. In some embodiments, R¹⁸ is OH.

In some embodiments, R¹⁵ is methoxy, and R¹⁶ is F. In some embodiments, R¹⁶ is OH, and R¹⁵ is fluoro or trifluoromethyl. In some embodiments, R¹⁷ and R¹⁸ are both H. In some embodiments, R¹⁷ and R¹⁸ are both OH. In some embodiments, provided is a compound of formula (III-1):

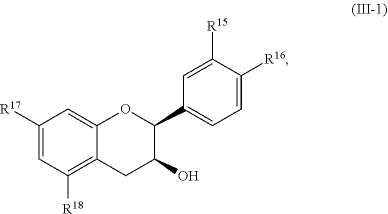

or a pharmaceutically acceptable salt, stereoisomer or tautomer thereof, wherein each variable is as defined for formula (III) or any variation thereof.

In some embodiments, provided is a compound of formula (III-2):

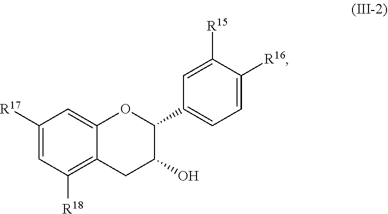

or a pharmaceutically acceptable salt, stereoisomer or tautomer thereof, wherein each variable is as defined for formula (III) or any variation thereof.

In some embodiments, provided is a compound of formula (III-3):

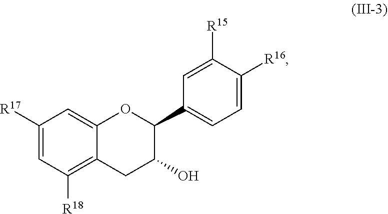

or a pharmaceutically acceptable salt, stereoisomer or tautomer thereof, wherein each variable is as defined for formula (III) or any variation thereof.

In some embodiments, provided is a compound of formula (III-4):

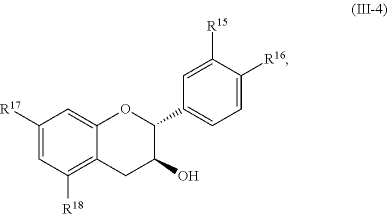

or a pharmaceutically acceptable salt, stereoisomer or tautomer thereof, wherein each variable is as defined for formula (III) or any variation thereof.

In some embodiments, the compound of Formula (III) is selected from the group consisting of:

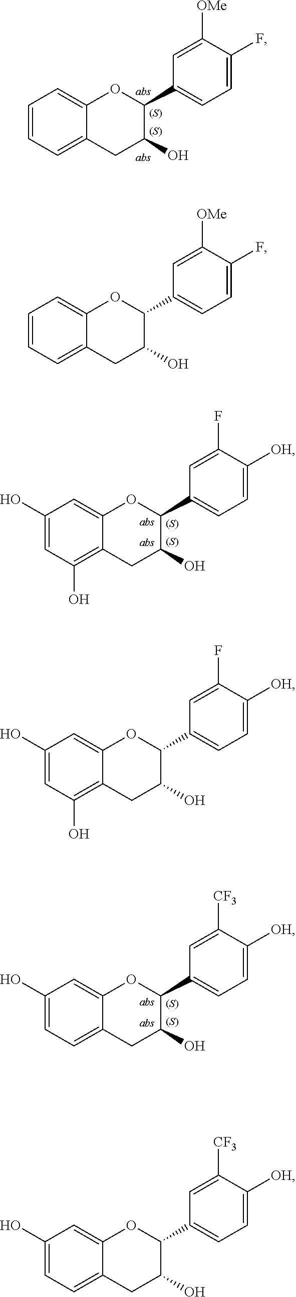

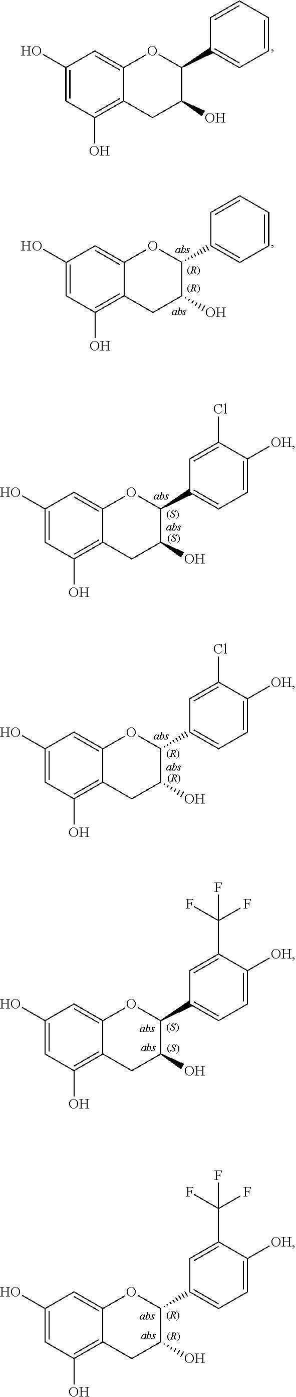

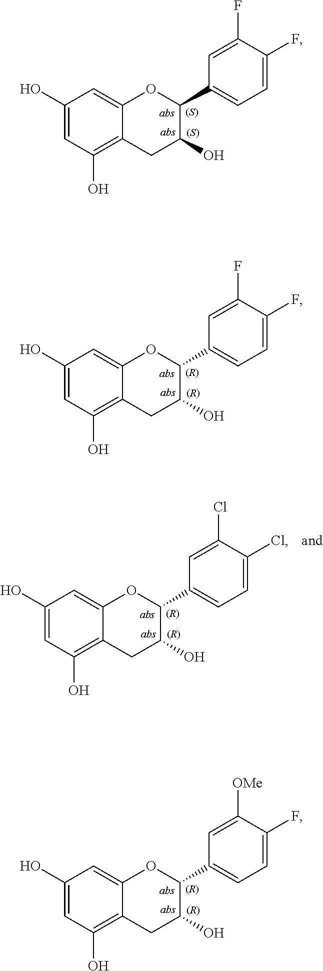

or a pharmaceutically acceptable salt, stereoisomer or tautomer thereof.

In some embodiments, the compound is

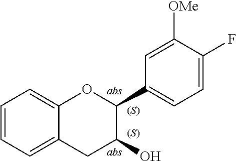

or a pharmaceutically acceptable salt, stereoisomer or tautomer thereof. In some embodiments, the compound is

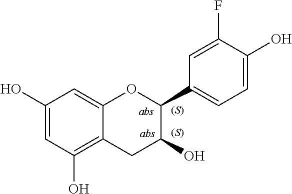

or a pharmaceutically acceptable salt, stereoisomer or tautomer thereof. In some embodiments, the compound is

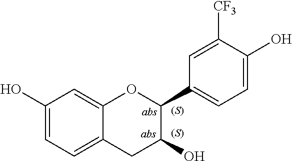

or a pharmaceutically acceptable salt, stereoisomer or tautomer thereof. In some embodiments, the compound is

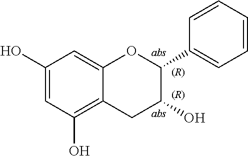

or a pharmaceutically acceptable salt, stereoisomer or tautomer thereof.

In some embodiments, the compound is not

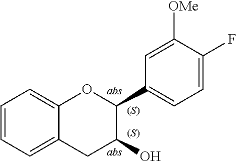

or a pharmaceutically acceptable salt, stereoisomer or tautomer thereof. In some embodiments, the compound is not

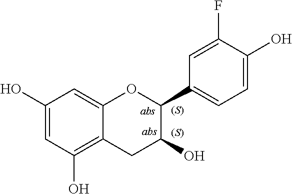

or a pharmaceutically acceptable salt, stereoisomer or tautomer thereof. In some embodiments, the compound is not

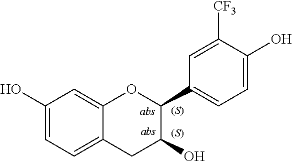

or a pharmaceutically acceptable salt, stereoisomer or tautomer thereof. In some embodiments, the compound is not

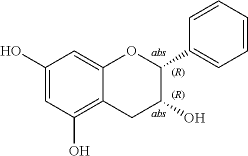

or a pharmaceutically acceptable salt, stereoisomer or tautomer thereof.

In some aspects, provided is a compound selected from the group consisting of

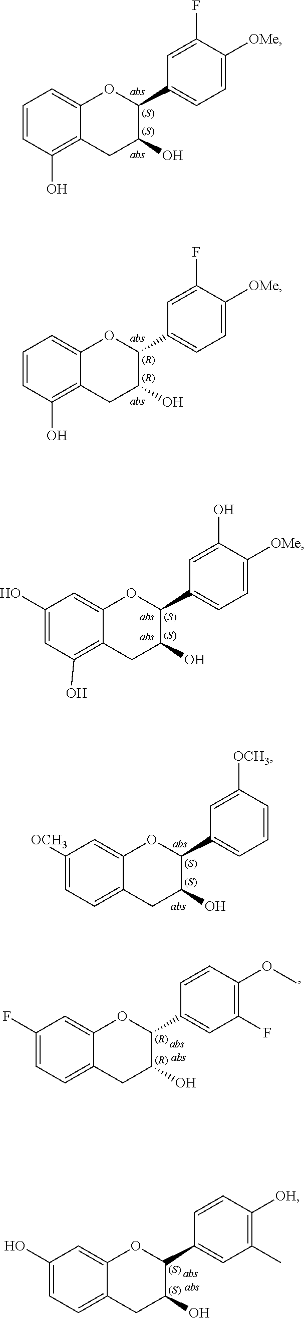

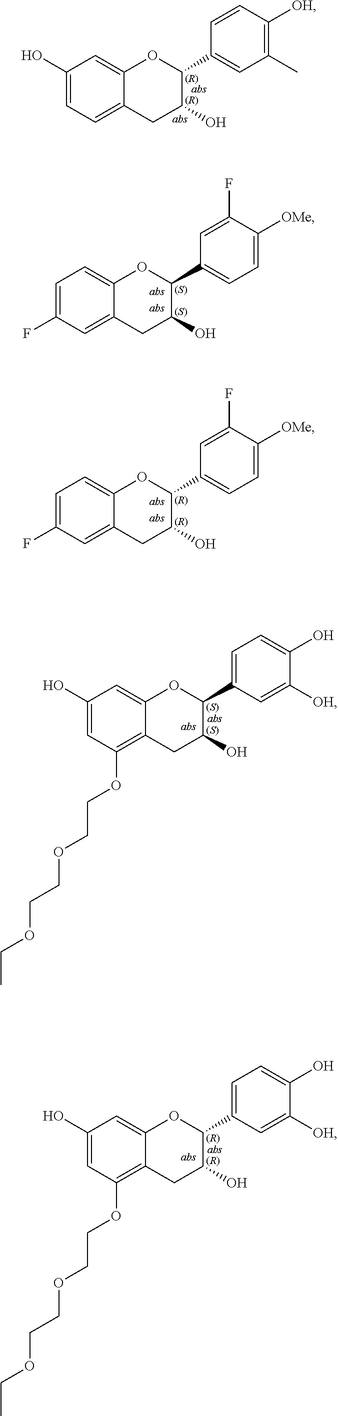

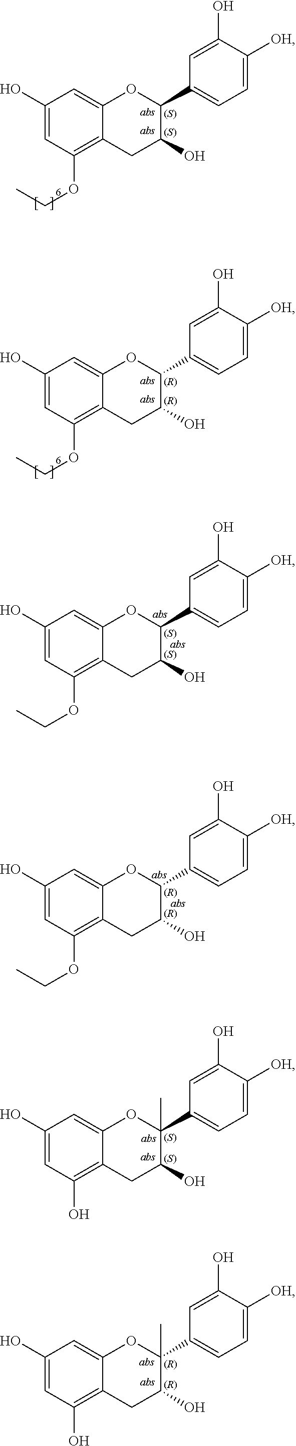

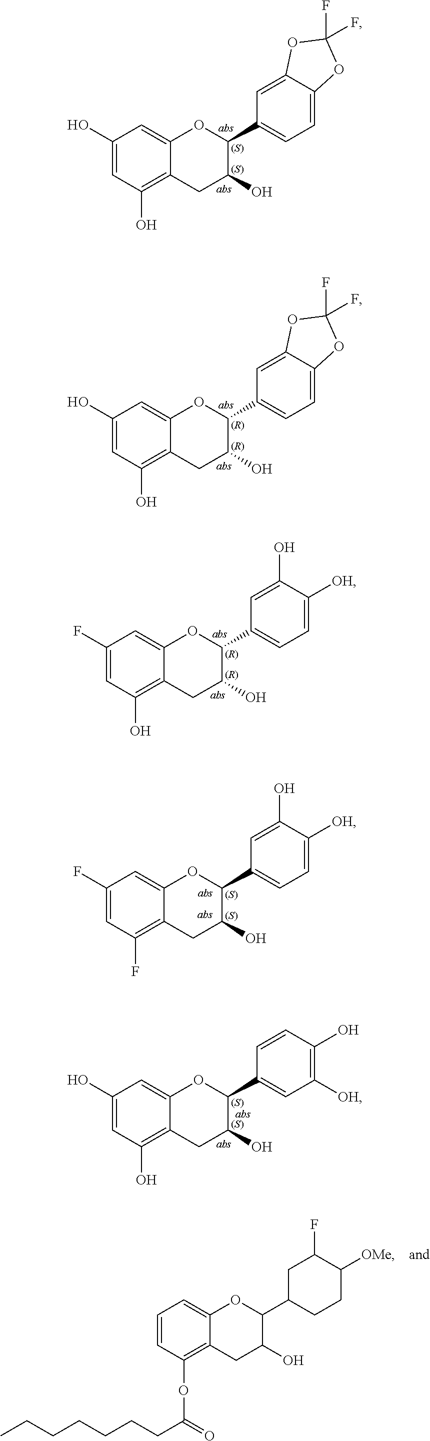

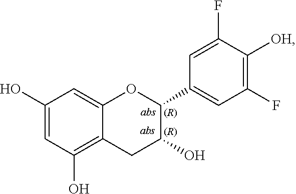

or a pharmaceutically acceptable salt, stereoisomer or tautomer thereof.

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to X¹ of formula (I) may be combined with every description, variation, embodiment or aspect of X², X³, X⁴, R⁴, X⁵, and/or R⁶ the same as if each and every combination were specifically and individually listed. Every description, variation, embodiment or aspect provided herein with respect to X of formula (II) may be combined with every description, variation, embodiment, or aspect of R⁷, R⁸, and/or R⁹ the same as if each and every combination were specifically and individually listed. Every description, variation, embodiment or aspect provided herein with respect to R¹⁵ of formula (III) may be combined with every description, variation, embodiment, or aspect of R¹⁶, R¹⁷, and/or R¹⁸ the same as if each and every combination were specifically and individually listed.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity. In some embodiments, the provided compounds are sterilized.

Representative compounds are listed in Table 1.

TABLE 1
Representative compounds.

| Comp. | | |
|---|---|---|
| Number | Structure | Chemical Name |
| 1 | 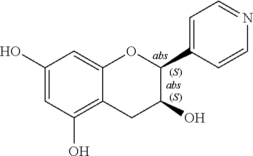 | (2S,3S)-2-(pyridin- 4-yl)-3,4-dihydro- 2H-1-benzopyran- 3,5,7-triol |
| 2 | 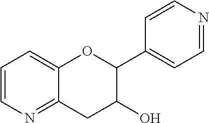 [SMILES: OC1Cc2ncccc2OC1c1ccncc1] | 2-(pyridin-4-yl)- 2H,3H,4H- pyrano[3,2- b]pyridin-3-ol |
| 3 | 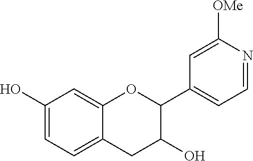 [SMILES: COc1cc(C2Oc3cc(O)ccc3CC2O)ccn1] | |
| 4 | 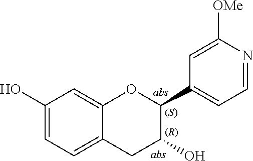 | |
| 5 | 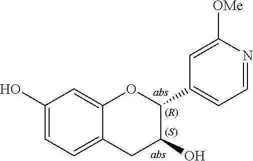 | |
| 6 | 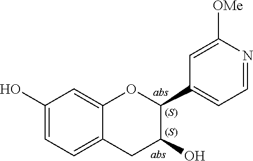 | |
| 7 | 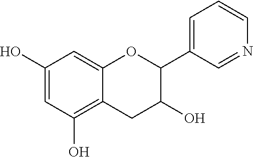 [SMILES: Oc1cc(O)c2c(c1)OC(c1cccnc1)C(O)C2] | |
| 8 | 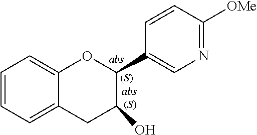 | |
| 9 | 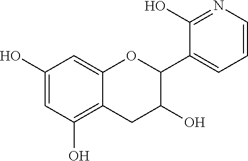 [SMILES: Oc1cc(O)c2c(c1)OC(c1cccnc1O)C(O)C2] | |
| 10 | 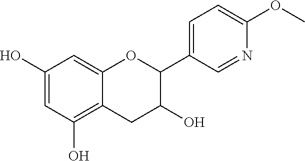 [SMILES: COc1ccc(C2Oc3cc(O)cc(O)c3CC2O)cn1] | |
| 11 | 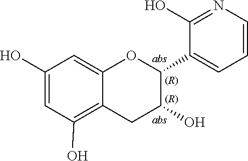 | (2R,3R)-2-(2- hydroxypyridin-3- yl)chromane-3,5,7- triol |
| 12 | 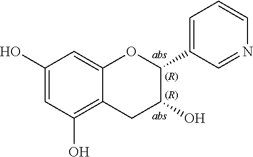 | |
| 13 | 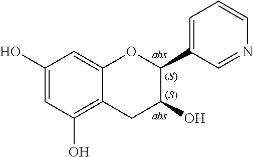 | |
| 14 | 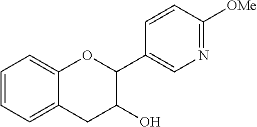 [SMILES: COc1ccc(C2Oc3ccccc3CC2O)cn1] | |
| 15 | 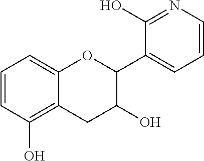 [SMILES: Oc1cccc2c1CC(O)C(c1cccnc1O)O2] | |
| 16 | 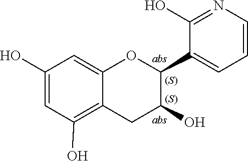 | |
| 17 | 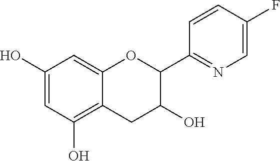 [SMILES: Oc1cc(O)c2c(c1)OC(c1ccc(F)cn1)C(O)C2] | |
| 18 | 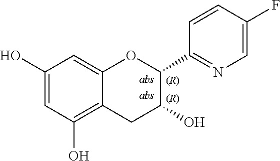 | (2R,3R)-2-(5- fluoropyridin-2- yl)chromane-3,5,7- triol |
| 19 | 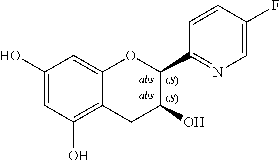 | (2S,3S)-2-(5- fluoropyridin-2- yl)chromane-3,5,7- triol |
| 20 | 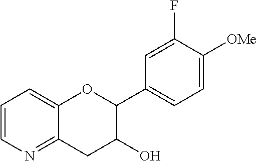 [SMILES: COc1ccc(C2Oc3cccnc3CC2O)cc1F] | |
| 21 | 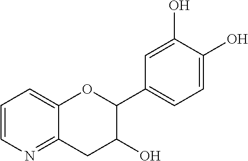 [SMILES: Oc1ccc(C2Oc3cccnc3CC2O)cc1O] | |
| 22 | 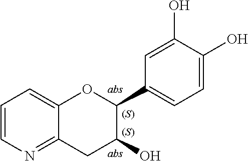 | (2S,3S)-4-(3- hydroxy-3,4- dihydro-2H- pyrano[3,2- b]pyridin-2- yl)benzene-1,2-diol |
| 23 | 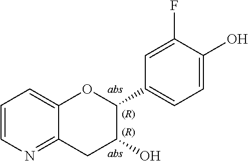 | |
| 24 | 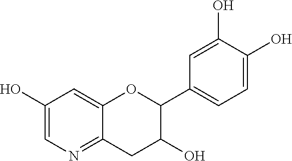 [SMILES: Oc1cnc2c(c1)OC(c1ccc(O)c(O)c1)C(O)C2] | |
| 25 | 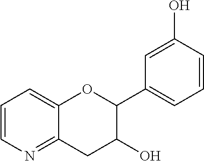 [SMILES: Oc1cccc(C2Oc3cccnc3CC2O)c1] | |
| 26 | 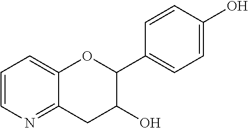 [SMILES: Oc1ccc(C2Oc3cccnc3CC2O)cc1] | |
| 27 | 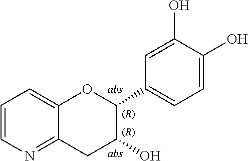 | (2R,3R)-4-(3- hydroxy-3,4- dihydro-2H- pyrano[3,2- b]pyridin-2- yl)benzene-1,2-diol |
| 28 | 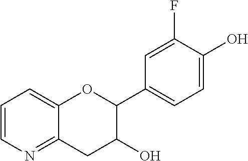 [SMILES: Oc1ccc(C2Oc3cccnc3CC2O)cc1F] | |
| 29 | 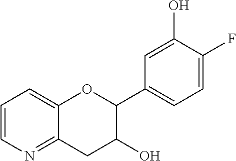 [SMILES: Oc1cc(C2Oc3cccnc3CC2O)ccc1F] | |
| 30 | 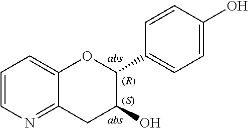 | |
| 31 | 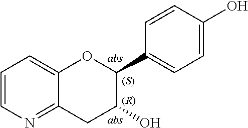 | |
| 32 | 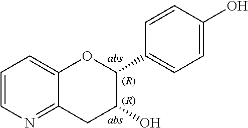 | |
| 33 | 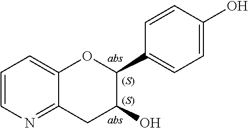 | |
| 34 | 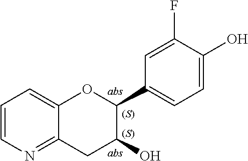 | (2S,3S)-2-(3-fluoro- 4-hydroxyphenyl)- 3,4-dihydro-2H- pyrano[3,2- b]pyridin-3-ol |
| 35 | 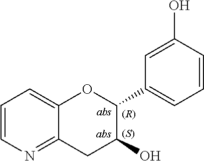 | |
| 36 | 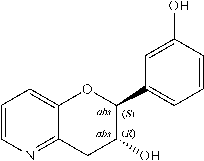 | (2S,3R)-2-(3- hydroxyphenyl)-3,4- dihydro-2H- pyrano[3,2- b]pyridin-3-ol |
| 37 | 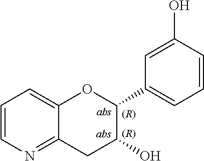 | (2R,3R)-2-(3- hydroxyphenyl)-3,4- dihydro-2H- pyrano[3,2- b]pyridin-3-ol |
| 38 | 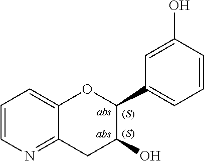 | |
| 39 | 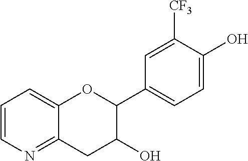 [SMILES: Oc1ccc(C2Oc3cccnc3CC2O)cc1C(F)(F)F] | |
| 40 | 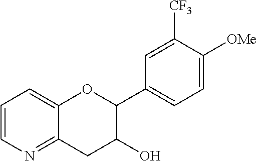 [SMILES: COc1ccc(C2Oc3cccnc3CC2O)cc1C(F)(F)F] | |
| 41 | 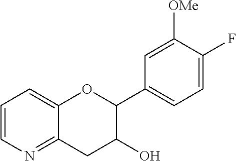 [SMILES: COc1cc(C2Oc3cccnc3CC2O)ccc1F] | |
| 42 | 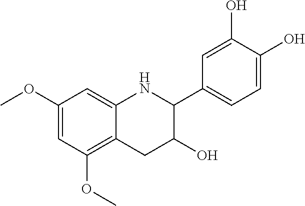 [SMILES: COc1cc2c(c(OC)c1)CC(O)C(c1ccc(O)c(O)c1)N2] | |
| 43 | 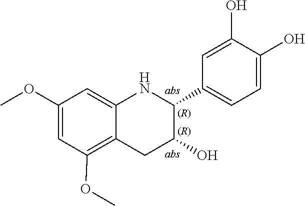 | (2R,3R)-4-(3- hydroxy-5,7- dimethoxy-1,2,3,4- tetrahydroquinolin- 2-yl)benzene-1,2- diol |
| 44 | 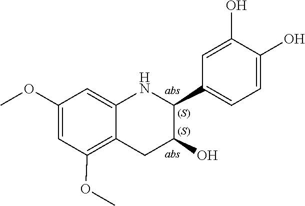 | |
| 45 | 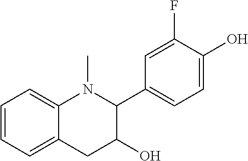 [SMILES: CN1c2ccccc2CC(O)C1c1ccc(O)c(F)c1] | |
| 46 | 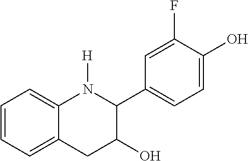 [SMILES: Oc1ccc(C2Nc3ccccc3CC2O)cc1F] | |
| 47 | 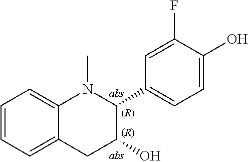 | |
| 48 | 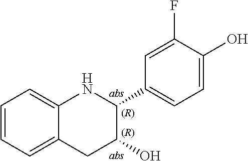 | |
| 49 | 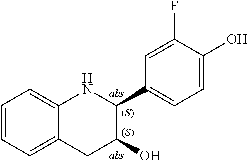 | (2S,3S)-2-(3-fluoro- 4-hydroxyphenyl)- 1,2,3,4- tetrahydroquinolin- 3-ol |
| 50 | 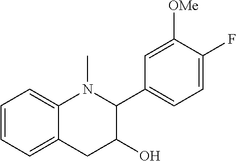 [SMILES: COc1cc(C2C(O)Cc3ccccc3N2C)ccc1F] | |
| 51 | 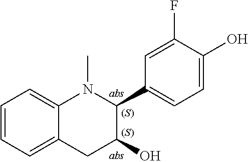 | (2S,3S)-2-(3-fluoro- 4-hydroxyphenyl)-1- methyl-1,2,3,4- tetrahydroquinolin- 3-ol |
| 52 | 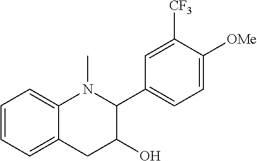 [SMILES: COc1ccc(C2C(O)Cc3ccccc3N2C)cc1C(F)(F)F] | |
| 53 | 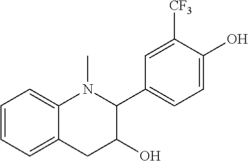 [SMILES: CN1c2ccccc2CC(O)C1c1ccc(O)c(C(F)(F)F)c1] | |
| 54 | 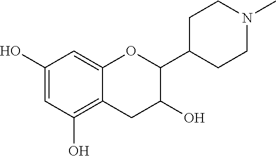 [SMILES: CN1CCC(C2Oc3cc(O)cc(O)c3CC2O)CC1] | |
| 55 | 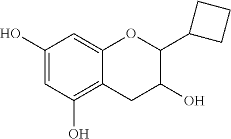 [SMILES: Oc1cc(O)c2c(c1)OC(C1CCC1)C(O)C2] | |
| 56 | 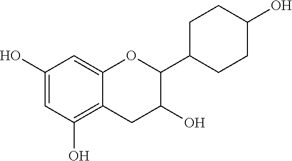 [SMILES: Oc1cc(O)c2c(c1)OC(C1CCC(O)CC1)C(O)C2] | |
| 57 | 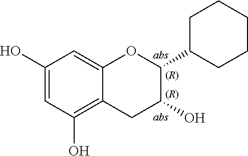 | (2R,3R)-2- cyclohexylchromane- 3,5,7-triol |
| 58 | 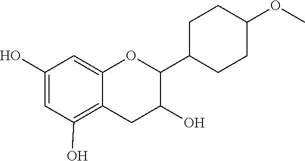 [SMILES: COC1CCC(C2Oc3cc(O)cc(O)c3CC2O)CC1] | |
| 59 | 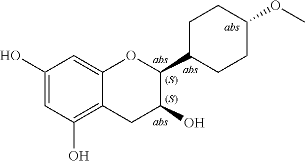 | |
| 60 | 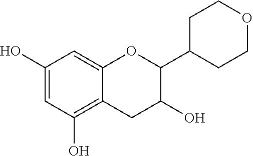 [SMILES: Oc1cc(O)c2c(c1)OC(C1CCOCC1)C(O)C2] | |
| 61 | 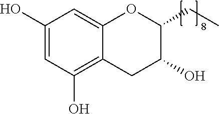 | (2R,3R)-2- nonylchromane- 3,5,7-triol |
| 62 | 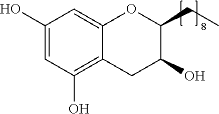 | (2S,3S)-2- nonylchromane- 3,5,7-triol |
| 63 | 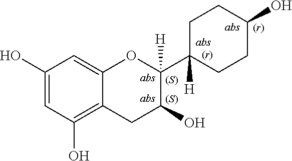 | |
| 64 | 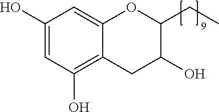 | |
| 65 | 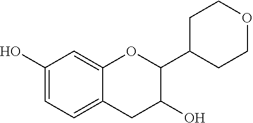 [SMILES: Oc1ccc2c(c1)OC(C1CCOCC1)C(O)C2] | |
| 66 | 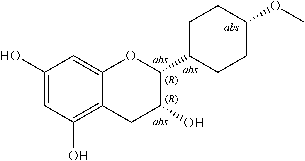 | |
| 67 | 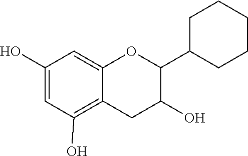 [SMILES: Oc1cc(O)c2c(c1)OC(C1CCCCC1)C(O)C2] | |
| 68 | 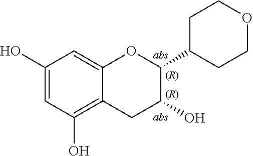 | (2R,3R)2- (tetrahydro-2H- pyran-4- yl)chromane-3,5,7- triol |
| 69 | 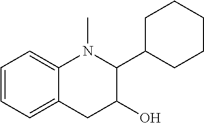 [SMILES: CN1c2ccccc2CC(O)C1C1CCCCC1] | |
| 70 | 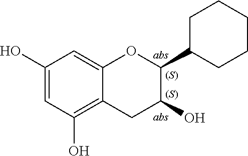 | (2S,3S)-2- cyclohexylchromane- 3,5,7-triol |
| 71 | 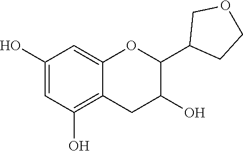 [SMILES: Oc1cc(O)c2c(c1)OC(C1CCOC1)C(O)C2] | |
| 72 | 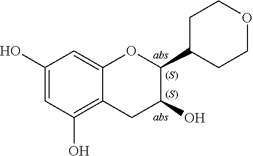 | (2S,3S)-2- (tetrahydro-2H- pyran-4- yl)chromane-3,5,7- triol |
| 73 | 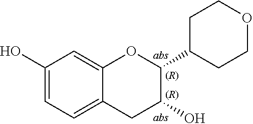 | |
| 74 | 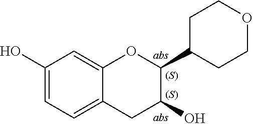 | |
| 75SS | 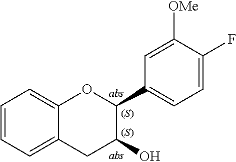 | (2S,3S)-2-(4-fluoro- 3- methoxyphenyl) chroman-3-ol |
| 75RR | 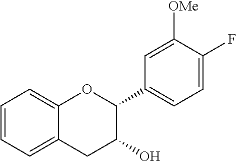 [SMILES: COc1cc([C@H]2Oc3ccccc3C[C@H]2O)ccc1F] | (2R,3R)-2-(4-fluoro- 3- methoxyphenyl) chroman-3-ol |
| 76SS | 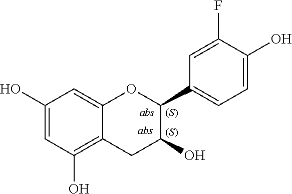 | (2S,3S)-2-(3-fluoro- 4- hydroxyphenyl) chromane-3,5,7-triol |
| 76RR | 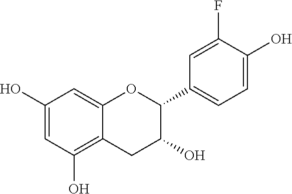 [SMILES: Oc1cc(O)c2c(c1)O[C@H](c1ccc(O)c(F)c1)[C@H](O)C2] | (2R,3R)-2-(3-fluoro- 4- hydroxyphenyl) chromane-3,5,7-triol |
| 77SS | 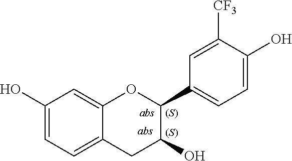 | (2S,3S)-2-(4- hydroxy-3- (trifluoromethyl) phenyl)chromane-3,7- diol |
| 77RR | 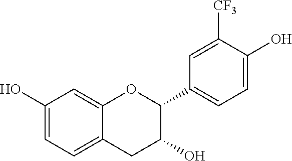 [SMILES: Oc1ccc2c(c1)O[C@H](c1ccc(O)c(C(F)(F)F)c1)[C@H](O)C2] | (2R,3R)-2-(4- hydroxy-3- (trifluoromethyl) phenyl)chromane-3,7- diol |
| 78SS | 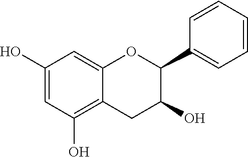 [SMILES: Oc1cc(O)c2c(c1)O[C@@H](c1ccccc1)[C@@H](O)C2] | (2S,3S)-2- phenylchromane- 3,5,7-triol |
| 78RR | 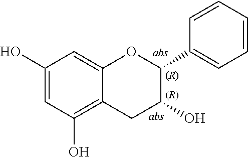 | (2R,3R)-2- phenylchromane- 3,5,7-triol |
| 79 | 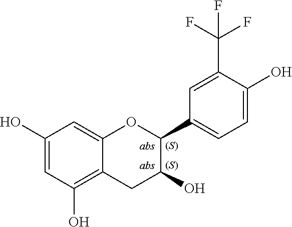 | |
| 80 | 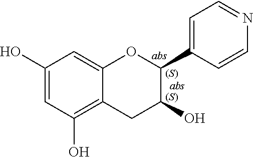 | |
| 81 | 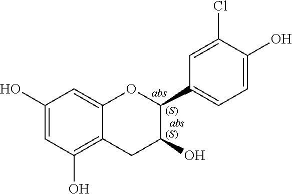 | |
| 82 | 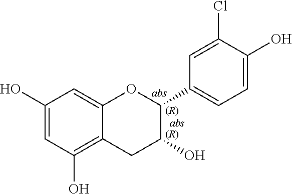 | |
| 83 | 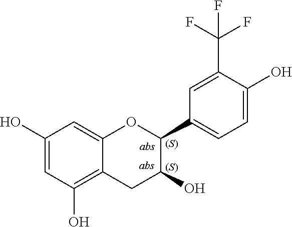 | |
| 84 | 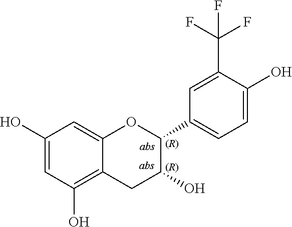 | |
| 85 | 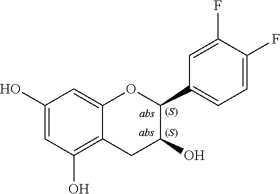 | |
| 86 | 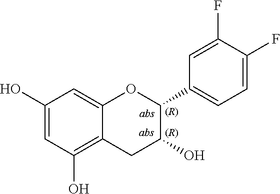 | |
| 87 | 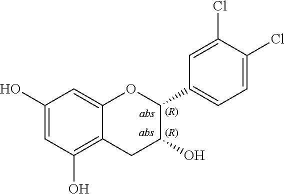 | |
| 88 | 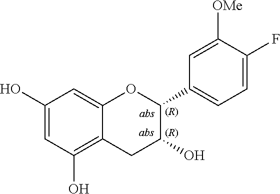 | |
| 89 | 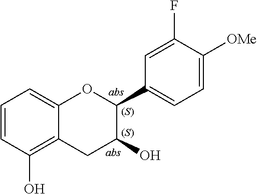 | |
| 90 | 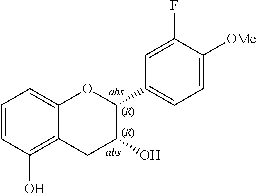 | |
| 91 | 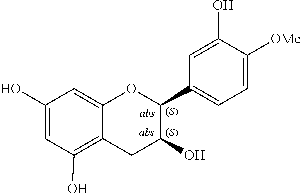 | |
| 92 | 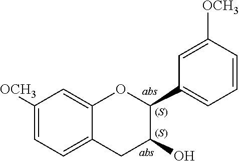 | |
| 93 | 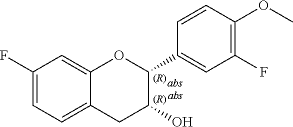 | |
| 94 | 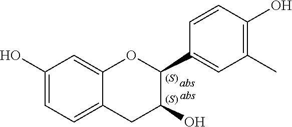 | |
| 95 | 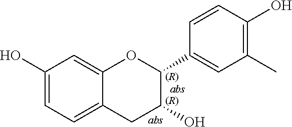 | |
| 96 | 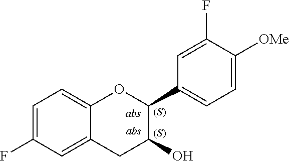 | |
| 97 | 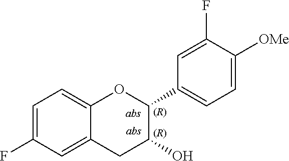 | |
| 98 | 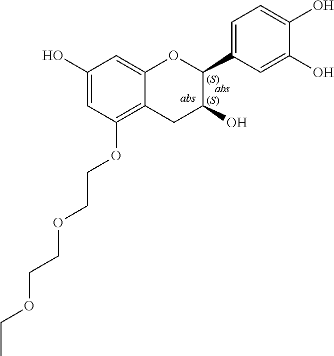 | |
| 99 | 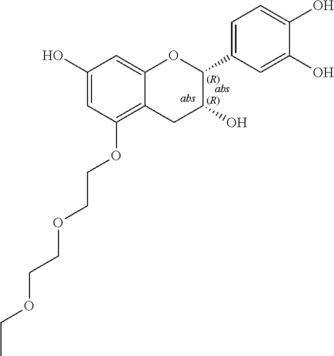 | |
| 100 | 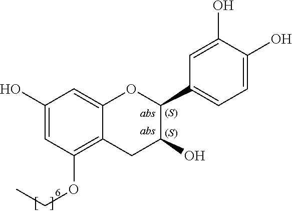 | |
| 101 | 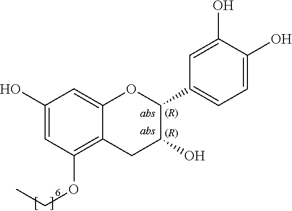 | |
| 102 | 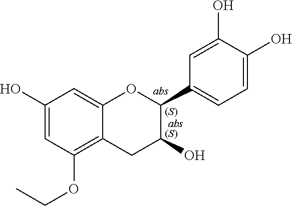 | |
| 103 | 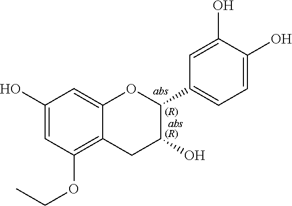 | |
| 104 | 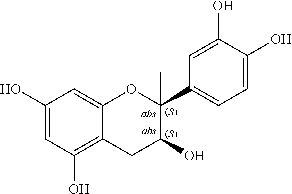 | |
| 105 | 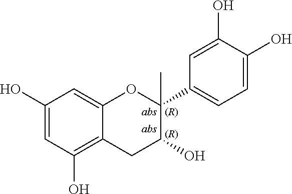 | |
| 106 | 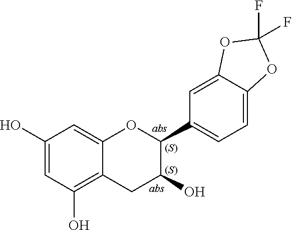 | |
| 107 | 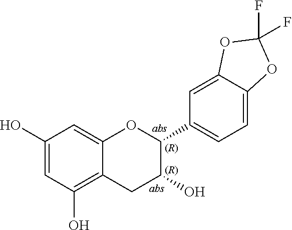 | |
| 108 | 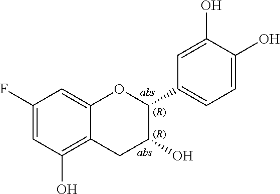 | |
| 109 | 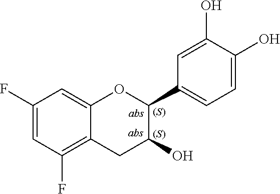 | |
| 110 | 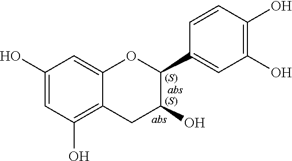 | |
| 111 | 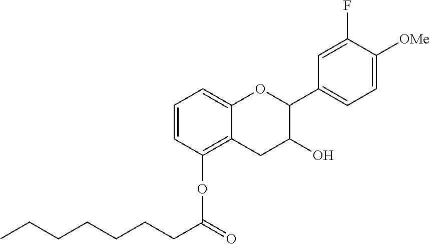 [SMILES: CCCCCCCC(=O)Oc1cccc2c1CC(O)C(c1ccc(OC)c(F)c1)O2] | |
| 112 | 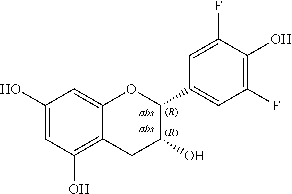 | |

Provided herein is a compound selected from the group consisting of the compounds described in Table 1.

Also provided herein are, where applicable, are any and all stereoisomers of the compounds depicted herein, including compounds of formulae (I), (II), and (III), including geometric isomers (e.g., cis/trans isomers or E/Z isomers), enantiomers, diastereomers, or mixtures thereof in any ratio, including racemic mixtures. In some embodiments, a provided compound has two stereocenters which are in the cis configuration. In some embodiments, a provided compound has two stereocenters which are in the trans configuration. In some embodiments, a provided compound has two stereocenters in the (S,S) configuration. In some embodiments, a provided compound has two stereocenters in the (R,R) configuration. In some embodiments, a provided compound has two stereocenters in the (S,R) configuration. In some embodiments, a provided compound has two stereocenters in the (R, S) configuration. In some embodiments, a provided compound is present at 75% stereoisomeric purity in a mixture of enantiomers and/or diastereomers. In some embodiments, a provided compound is present at 80% stereoisomeric purity in a mixture of enantiomers and/or diastereomers. In some embodiments, a provided compound is present at 90% stereoisomeric purity in a mixture of enantiomers and/or diastereomers. In some embodiments, a provided compound is present at 95% stereoisomeric purity in a mixture of enantiomers and/or diastereomers. In some embodiments, a provided compound is present at 99% stereoisomeric purity in a mixture of enantiomers and/or diastereomers. In some embodiments, a provided compound is present at 99.5% stereoisomeric purity in a mixture of enantiomers and/or diastereomers. In some embodiments, a provided compound is present at 99.9% stereoisomeric purity in a mixture of enantiomers and/or diastereomers. In some embodiments, a provided compound is a single stereoisomer that is substantially free of other enantiomers and/or diastereomers.

It is understood that compounds with tautomeric forms are described and embraced herein. Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The compounds described here also intend isotopically-labeled and/or isotopically-enriched forms. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into the provided compounds include isotopes of hydrogen, carbon, nitrogen, oxygen, phosphorus, sulfur, chlorine, such as ²H, ³H, ¹¹C, ¹³C, ¹⁴C ¹³N, ¹⁵O, ¹⁷O, ³²P, ³⁵S, ¹⁸F, ³⁶Cl. Certain isotope labeled compounds (e.g. ³H and ¹⁴C) is useful in compound or substrate tissue distribution studies. Incorporation of heavier isotopes such as deuterium (²H) can afford certain therapeutic advantages resulting from greater metabolic stability, for example, increased in vivo half-life, or reduced dosage requirements and, hence may be preferred in some instances.

Isotopically-labeled compounds can generally be prepared by standard methods and techniques known to those skilled in the art or by procedures similar to those described in the accompanying Examples substituting appropriate isotopically-labeled reagents in place of the corresponding non-labeled reagent.

Compositions of any of the compounds detailed herein are embraced by this disclosure. In some embodiments, the provided is pharmaceutical compositions comprising a compound as detailed herein or a salt thereof and a pharmaceutically acceptable carrier or excipient.

### Methods of Preparation

The provided compounds may be prepared by a number of processes, including but not limited to the processes generally described below. In the following process descriptions, the symbols when used in the formulae depicted are to be understood to represent those groups described above in relation to the formulae herein.

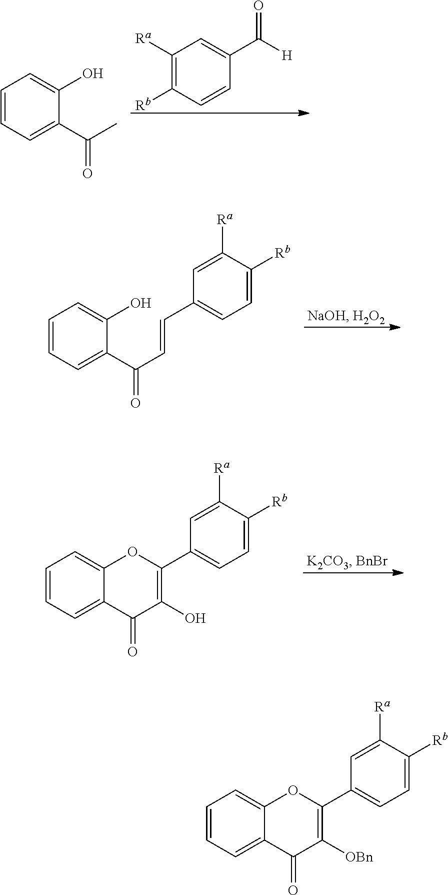

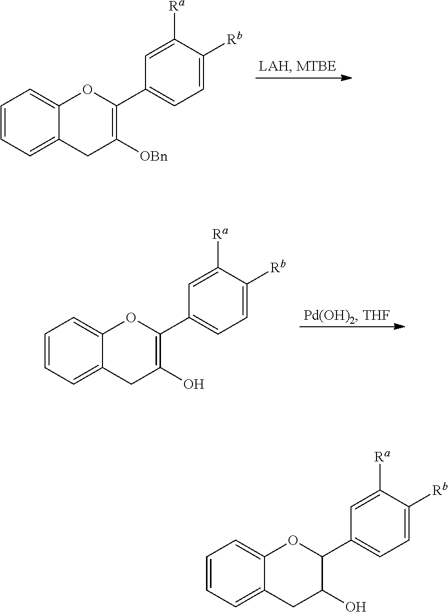

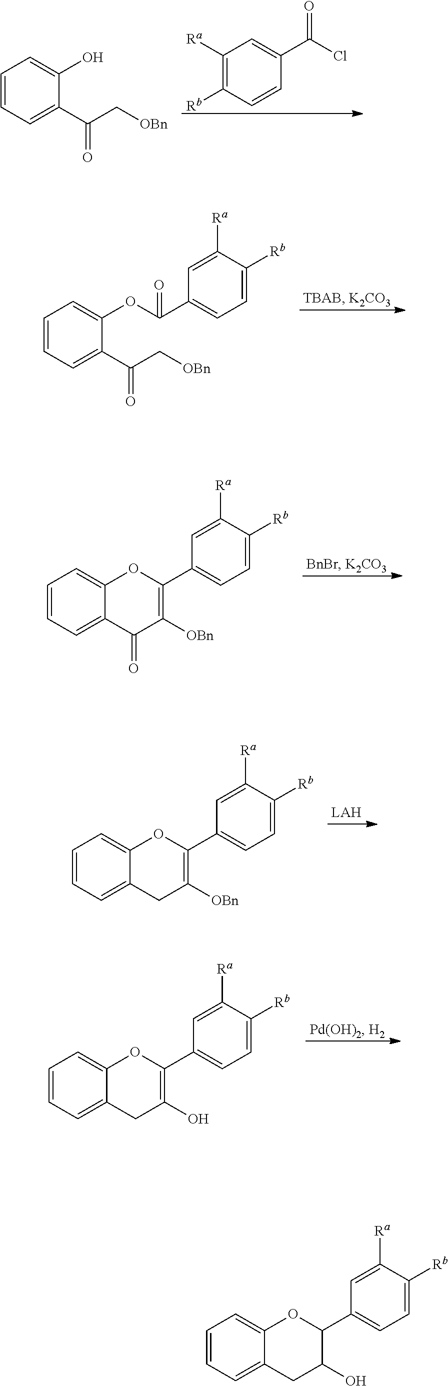

Similar methods may be used to access compounds of Formula (I) and Formula (II).

### Methods of Use

The provided compounds and compositions can be used to improve the physicochemical properties of pharmaceutical and nutraceutical ingredients.

The compounds and compositions described herein may be used for all indications in which epicatechin is indicated, including, without limitation, any of the diseases or conditions described in WO2012/170430, WO2013/022846, WO2013/142816, US2018/0193306, WO2014/162320, WO2017/221269, and WO2018/083713, each of which is hereby incorporated by reference in its entirety.

In yet another aspect, provided is methods for treating diseases or disorders that would benefit from increased expression of Electron transfer Chain (ETC), particularly ETC IV. The methods involve administering to a subject in need thereof a therapeutically effective amount of the compounds and compositions described herein.

The vast majority of the body's need for ATP is supplied through the process of oxidative phosphorylation, carried out in the mitochondria in all tissues. There are 5 protein complexes, known as the Electron Transport Complexes that effect ATP synthesis. ETC I, II, III, and IV mediate electron transport. ETC I, III, and IV also function as proton pumps that maintain an electrochemical gradient necessary for activity of ETC V, the ATP synthase enzyme that makes ATP from ADP. Complex I, also known as cytochrome c oxidase, (COX), consists of 14 subunits whose assembly into a functional complex requires an additional 30 protein factors. ETC IV is particularly important to oxidative phosphorylation. It is the only one of the ETC complexes to manifest tissue-specific and developmentally regulated isoforms, allowing precise regulation of oxidative phosphorylation under a variety of metabolic demands. Thus the ETC IV (COX) protein complex is considered to be the rate-limiting step in oxidative phosphorylation. Small positive or negative changes in ETC IV can exert a significant impact on health. Selective activation of COX activity has been associated with improved cognition, improved neuronal cell survival under stress, and improved wound healing. Mutations in the numerous proteins that comprise or regulate the activity of ETC IV reveal the pathological consequences of even modest decreases in ETC IV activity. As little as a 30% reduction in COX activity has been shown to induce cardiomyopathy or be associated with the development of neurodegenerative diseases such as Alzheimer's. Decreases in COX (ETCIV) expression due to mutations or molecular manipulation have been associated with loss of muscle endurance and speed, muscle dystonia, immunodeficiency states due to impaired T cell maturation, cardiomyopathy, particularly of the aging phenotype, ataxia, neurodegeneration, increased toxicity in the setting of ischemia, pulmonary inflammation and fibrosis, encephalopathy, vascular insufficiency, and stimulation of cancer cell proliferation. Additional specific diseases associated with COX subunit isoform mutations causing loss of function include exocrine pancreatic insufficiency, inflammatory lung disease, Charcot-Marie-Tooth disease, infantile encephalomyopathy, and Leigh syndrome neurodegeneration with epilepsy.

The following conditions associated with loss of COX expression or function would be expected to be therapeutically responsive to a potent, preferential inducer of COX (ETC IV) expression: impaired cognition, neurodegenerative diseases such as Alzheimer's or Leigh syndrome, dystonia, sarcopenia, cardiomyopathy of aging or other diseases associated with mitochondrial dysfunction, ischemic vascular disease, immunodeficiency states, ataxia, pulmonary inflammation and fibrosis, infantile encephalomyopathy, epilepsy, Charcot-Marie-Tooth disease, exocrine pancreatic insufficiency, impaired wound healing, growth of cancer cells.

In some embodiments, the compounds and compositions described herein may be used for inducing mitochondrial biogenesis, including biogenesis of any one or more of ETC I, II, III, IV, and V.

In addition, epicatechin can be used in lowering the elevated triglycerides. In some embodiments, the compounds and compositions described herein may be use in medicament for conditions associated with elevated triglycerides, such as metabolic syndrome, Type II diabetes, congenital hyperlipidemias, and drug-induced hyperlipidemia, as is observed with corticosteroid treatments.

In another embodiment, provided are methods for prophylactic and/or therapeutic treatment of conditions related to mitochondrial dysfunction resulting from administration of one or more chemical compositions that exhibit mitochondrial toxicity. In some embodiments, the mitochondrial toxicity is identified based on or associated with one or more biological effects, which include, but are not limited to, abnormal mitochondrial respiration, abnormal oxygen consumption, abnormal extracellular acidification rate, abnormal mitochondrial number, abnormal lactate accumulation, and abnormal ATP levels. In some embodiments, the mitochondrial toxicity is identified based on or associated with one or more physiological manifestations, which include, but are not limited to, elevations in markers known to relate to injury to the heart, liver, and/or kidney, elevated serum liver enzymes, elevated cardiac enzymes, lactic acidosis, elevated blood glucose, and elevated serum creatinine. In another embodiment, provided are methods for treating chronic mitochondrial depletion and the symptoms arising as a result of drug-associated toxicity or as a combination of drug associated toxicity occurring within a background of biological depletion of mitochondrial number, as occurs in diabetes, obesity, and during the course of aging. In another embodiment, provided are methods for treating chronic perturbation of mitochondria function or structure, including chronic myopathy, sarcopenia, persistent diabetes, chronic fatigue syndromes, gastrointestinal symptoms, liver, and cardiovascular dysfunction and failure, neurological symptoms, impaired sleep, and persistent alteration in cognitive acuity or function, such as memory.

In another embodiment, provided are methods for treating, preventing, or reversing injury to skeletal or cardiac muscles, for treating or preventing diseases relating to the structure and function of skeletal or cardiac muscles, and for inducing regeneration or restructuring of skeletal or cardiac muscle as a means for treating disease relating to abnormalities in the skeletal or cardiac muscle structure and function in a subject.

In some embodiments, provided are methods for treatment of impaired skeletal or cardiac muscle function due to aging, obesity, disuse or inactivity, exposure to potentially toxic nutritional agents such as fructose, or exposure to inadequate nutrition such as starvation or malnutrition.

In some embodiments, provided are methods for the treatment of muscle-related side effects of athletic training or competition including soreness, cramping, weakness, pain, or injury.

In some embodiments, provided are methods for the treatment of skeletal or cardiac muscle diseases associated with ischemia, or impaired or inadequate blood flow. In some embodiments, the diseases are selected from the group consisting of atherosclerosis, trauma, diabetes, vascular stenosis, peripheral arterial disease, vasculopathy, and vasculitis.

In some embodiments, provided are methods for the treatment of diseases associated with genetic disorders that directly or indirectly affect the number, structure, or function of cardiac muscle cells or skeletal muscle cells. In some embodiments, the disease is selected from the group consisting of muscular dystrophies and Friedreich's ataxia.

In some embodiments, provided are methods for the treatment of diseases associated with impaired neurological control of muscular activity resulting in consequent abnormalities in structure and function of skeletal muscles due to inactivity, aberrant contractility, or contracted states. In some embodiments, the disease is selected from the group consisting of peripheral denervation syndromes, trauma, amyotrophic lateral sclerosis, meningitis, and structural abnormalities of the spine, whether congenital or acquired.

In some embodiments, provided are methods for the treatment of diseases associated with loss of number, loss of function, or loss of correct, optimally efficient internal organization of skeletal muscle cells or cardiac muscle cells. In some embodiments, the disease is muscle wasting. In some embodiments, the disease is sarcopenia. In some embodiments, sarcopenia is associated with a variety of disorders, including aging, diabetes, abnormal metabolic conditions, infection, inflammation, autoimmune disease, cardiac dysfunction, arthritis congestive heart failure, aging, myocarditis, myositis, polymyalgia rheumatica, polymyositis, HIV, cancer, side effects of chemotherapy, malnutrition, aging, inborn errors of metabolism, trauma, stroke, and neurological impairment.

In some embodiments, the method of treating diseases associated with loss of number, loss of function, or loss of correct, optimally efficient internal organization of skeletal muscle cells or cardiac muscle cells further comprises exercise or programmatic sequences or intensities of exercise.

In some embodiments, provided are methods for enhancing sports performance, endurance, building muscle shape or strength, or facilitating recovery from the effects of training or competition.

In some embodiments, provided are methods for treating muscle injury, weakness, or pain associated with the administration of medicines. In some embodiments, provided are methods for use to prevent, ameliorate, or reverse muscle injury associated with medicines that damage mitochondria and/or cause myopathy as a secondary consequence.

In some embodiments of any one of the embodiments disclosed above, the skeletal or cardiac muscle injury of dysfunction in the subject is identified based on or associated with one or more physiological manifestations, which include, but are not limited to, elevated plasma levels of cardiac or skeletal muscle enzymes or proteins, such as myoglobin, troponin, or creatine phosphokinase, lactic acidosis, and elevated serum creatinine.

In some embodiments, provided are methods for stimulating the increased number or function of skeletal muscle cells or contractile muscle cells. Such stimulation of muscle cells may comprise stimulation of one or more aspects of muscle cell function, including cell division, muscle cell regeneration, activation of muscle satellite cells and their differentiation into adult muscle cells, recovery from injury, increased number or function of mitochondria or processes serving mitochondrial function, increased expression of proteins contributing to contractility, regulation of biochemical or translational processes, mitoses, or transduction of mechanical energy via dystrophin or other attachment processes. The methods and compositions described herein can assist in prevention of the consequences of muscle injury or dysfunction which have not yet occurred, as well as provide for the active therapy of muscle injury, dysfunction, or diseases which have already occurred.

In some embodiments, provided are methods of using muscle proteins whose expression is stimulated by administration of the compounds and compositions described herein as diagnostic biomarkers by which to determine the time and degree of muscle response to the therapeutic methods and compositions disclosed herein. Such biomarkers may be determined by measuring in tissue, plasma, blood, or urine the proteins themselves or the DNA or RNA nucleotides that encode for the proteins. In one embodiment, a decrease in the body of useful muscle proteins, such as dystrophin, or the presence of inhibitory proteins, such as thromobospondin, may be used to diagnose the severity of the abnormality of cardiac muscle structure or function or the probability of response to the therapeutic methods and compositions described herein. In another embodiment, changes in the levels of such biomarkers may be used to gauge the success or failure of certain therapeutic modalities, including those disclosed herein, in order to optimize the dose and to decide whether to maintain or change therapeutic methods and compositions.

In some embodiments, provided are methods of inducing follistatin production, inhibiting myostatin production, and/or increasing the ratio of follistatin to myostatin. This may be, for example, in associated with treating a muscle or bone considition or disorder.

Additional embodiments disclosed herein relate to a method to induce the increased cellular or muscular or bodily production of follistatin and follistatin-like proteins in order to reverse or ameliorate weakness of bone, thus preventing bone fractures, which may in some instances be caused by administration of compounds known to induce weakness of or damage to bone, impairment of bone generation, or impairment of bone growth, including but not limited to corticosteroids such as prednisone or deflazacort, anticonvulsants such as phenytoin and phenobarbital, chemotherapeutics such as aromatase inhibitors, and progestins. Further methods relate to inducing the increased cellular or muscular or bodily production of follistatin or follistatin-like proteins in order to reverse or ameliorate weakness of bone strength, thus preventing bone fractures, which may in some instances be associated with genetic predisposition, aging, inactive lifestyle, or low estrogen states such as menopause or post oophorectomy; a method to induce the increased cellular or muscular or bodily production of follistatin or follistatin-like proteins in order to reverse or ameliorate weakness of bone caused by medical conditions known to be associated with weakness of, or damage to, bone, impairment of bone generation, or impairment of bone growth, such celiac disease, kidney or liver disease, and immunomodulatory diseases such as systemic lupus erythematosus and rheumatoid arthritis; a method to induce the increased cellular or muscular or bodily production of follistatin or follistatin-like proteins in order to reverse or ameliorate weakness of bone in conjunction with the administration of other agents used to treat osteoporosis including calcium, Vitamin D, and calcitonin, in order to prevent bone fractures; a method to method to induce increased cellular or muscular or bodily production of follistatin or follistatin-like proteins as a therapeutic to accelerate the healing of bone fractures or to increase the degree of recovery from a bone fracture, such as those experienced in accidents, athletics, or combat; and a method to induce increased cellular or muscular or bodily production of follistatin or follistatin-like proteins in order to prevent systemic loss of bone density, and thus prevent subsequent bone fractures, during the recovery period after orthopedic surgery or after the onset of a disease or condition necessitating long periods of bed rest or physical inactivity, which are known to result in decreased bone density and muscle weakness.

In some embodiments, provided are methods for treating or preventing neurodegenerative diseases, such as amyotrophic lateral sclerosis (ALS), Parkinson's disease, Huntington's disease, spinal cord injury or abnormality, and peripheral and central neuropathies.

In some embodiments, provided are methods for treating or preventing celiac disease, kidney disease, liver disease, inflammatory diseases such as systemic lupus erythematosus and rheumatoid arthritis, osteoporosis, and bone fracture.

Conditions that may be treated by the compounds, compositions, and methods provided herein include: impaired skeletal and cardiac muscle function, recovery of skeletal or cardiac muscle health or function, functionally significant regeneration of skeletal or cardiac muscle cells or function.

In some embodiments, provided are methods for treating acute coronary syndromes, including but not limited to myocardial infarction and angina; acute ischemic events in other organs and tissues, renal injury, renal ischemia and diseases of the aorta and its branches; injuries arising from medical interventions, including but not limited to coronary artery bypass grafting (CABG) procedures and aneurysm repair; cancer; and metabolic diseases, diabetes mellitus and other such disorders.

In some embodiments, provided are methods for treating or preventing dystrophinopathy, such as Duchenne muscular dystrophy, Becker muscular dystrophy, and DMD-associated cardiomyopathy.

In some embodiments, provided are methods for treating or preventing sarcoglycanopathy, including α-sarcoglycanopathy (LGMD2D), β-sarcoglycanopathy (LGMD2E), γ-sarcoglycanopathy (LGMD2C), δ-sarcoglycanopathy (LGMD2F) and ε-sarcoglycanopathy (myoclonic dystonia). Sarcoglycanopathies include four subtypes of autosomal recessive limb-girdle muscular dystrophy (LGMD2C, LGMD2D, LGMD2E, and LGMD2F) that are caused, respectively, by mutations in the SGCG, SGCA, SGCB, and SGCD genes.

In some embodiments, provided are methods for treating or preventing dysferlinopathy, such as Miyoshi myopathy, scapuloperoneal syndrome, distal myopathy with anterior tibial onset, and elevated level of muscular enzyme CK.

Provided is a method of treating or preventing any of the diseases or conditions described herein in a subject in need thereof, comprising administering to the subject a therapeutically effective amount of the compounds and compositions described herein. Also provided are the compounds and compositions described herein for use the manufacture of a medicament for treating or preventing any of the diseases or conditions described herein in a subject in need thereof. Also provided are the compounds and compositions described herein for use in treating or preventing a disease or condition described herein in a subject in need thereof. Also provided are the compounds and compositions described herein for use in medical therapy. Also provided is use of the compounds and compositions described herein for treating or preventing a disease or condition described herein in a subject in need thereof.

In some aspects, provided herein is a method of treating or preventing a disease or disorder that would benefit from inhibition of ATP hydrolysis in a subject in need thereof, comprising administering to the subject a therapeutically effective amount of a provided compound or a pharmaceutically acceptable salt thereof. In some embodiments, the inhibition of ATP hydrolysis does not block mitochondrial ATP synthesis (e.g., complex V ATP synthesis).

In some embodiments, the disease or disorder causes metabolic dysfunction. In some embodiments, the disease or disorder causes impaired mitochondrial respiration. In some embodiments, the disease or disorder causes mitochondrial toxicity. In some embodiments, the mitochondrial toxicity is identified based on or associated with one or more biological effects, which include, but are not limited to, abnormal mitochondrial respiration, abnormal oxygen consumption, abnormal extracellular acidification rate, abnormal mitochondrial number, abnormal lactate accumulation, and abnormal ATP levels. In some embodiments, the mitochondrial toxicity is identified based on or associated with one or more physiological manifestations, which include, but are not limited to, elevations in markers known to relate to injury to the heart, liver, and/or kidney, elevated serum liver enzymes, elevated cardiac enzymes, lactic acidosis, elevated blood glucose, and elevated serum creatinine. Methods for assessing such biological effects or markers are known in the art and may be used in connection with the embodiments described herein. In some embodiments, the disease or disorder deceases mitochondrial ATP synthesis. In some embodiments, the disease or disorder increases ATP hydrolysis.

In some aspects, provided herein is a method of treating or preventing a condition related to acute or chronic excessive glutamate exposure in a subject in need thereof, comprising administering to the subject a therapeutically effective amount of epicatechin or a pharmaceutically acceptable salt thereof.

As used herein, the phrase “excessive glutamate exposure” refers to an amount of glutamate that induces excessive stimulation of glutamate receptors. In some embodiments, the excessive glutamate exposure results from increased extracellular glutamate concentrations. In some embodiments, the excessive glutamate exposure results from excessive glutamate release from the presynaptic membrane. In some embodiments, the excessive glutamate exposure results from impaired glutamate reuptake function. In some embodiments, the excessive glutamate refers to glutamate levels that are increased by greater than any of about 0.05-fold, 0.1-fold, 0.5-fold, 1-fold, 2-fold, 3-fold, 4-fold, 5-fold, or more, compared to the glutamate levels prior to excitotoxicity. In some embodiments, the excessive glutamate refers to glutamate levels that are increased by less than any of about 5-fold, 4-fold, 3-fold, 2-fold, 1-fold, 0.5-fold, 0.1-fold, 0.05-fold, or less, compared to the glutamate levels prior to excitotoxicity.

In some embodiments, the excessive glutamate exposure causes glutamate excitotoxicity. Glutamate excitotoxicity is the excessive stimulation of glutamate receptors, such as N-methyl-D-aspartate (NMDA) receptors, α-amino-3-hydroxy-5-methyl-4-isoxazolepropionic acid (AMPA) receptors, and/or kainate receptors. In some embodiments, glutamate excitotoxicity may induce neuronal degeneration, such as degeneration of dopaminergic neurons, thereby resulting in motor dysfunction. In some embodiments, the excessive glutamate exposure causes excessive stimulation of glutamate receptors.

In some embodiments, the excessive glutamate exposure increases intracellular sodium (Na²⁺) buildup. In some embodiments, the excessive glutamate exposure induces intracellular calcium (Ca²⁺) buildup. In some embodiments, the intracellular Ca²⁺ and/or Na²⁺ is increased by greater than any of about 0.05-fold, 0.1-fold, 0.5-fold, 1-fold, 2-fold, 3-fold, 4-fold, 5-fold, or more, compared to the intracellular Ca²⁺ and/or Na²⁺ prior to the glutamate exposure. In some embodiments, the intracellular Ca²⁺ and/or Na²⁺ is increased less than about 5-fold, 4-fold, 3-fold, 2-fold, 1-fold, 0.5-fold, 0.1-fold, 0.05-fold, or less, compared to the intracellular Ca²⁺ and/or Na²⁺ prior to the glutamate exposure. In some embodiments, the intracellular Ca²⁺ and/or Na²⁺ buildup may cause neuronal damage. In some embodiments, the intracellular Ca²⁺ and/or Na²⁺ buildup may cause cell death.

In some embodiments, the condition is selected from the group consisting of status epilepticus, neuroinflammatory disorders, pediatric seizure disorders, neuronal exocitoxicity, over activation of the NMDA receptor, post-operative syndromes of cognition loss, and loss of synaptic density.

### Dosages

The compounds and compositions disclosed and/or described herein are administered at a therapeutically effective dosage, e.g., a dosage sufficient to provide treatment for the disease state. While human dosage levels have yet to be optimized for the chemical entities described herein, generally, a daily dose ranges from about 0.01 to 100 mg/kg of body weight; in some embodiments, from about 0.05 to 10.0 mg/kg of body weight, and in some embodiments, from about 0.10 to 1.4 mg/kg of body weight. Thus, for administration to a 70 kg person, in some embodiments, the dosage range would be about from 0.7 to 7000 mg per day; in some embodiments, about from 3.5 to 700.0 mg per day, and in some embodiments, about from 7 to 100.0 mg per day. The amount of the chemical entity administered will be dependent, for example, on the subject and disease state being treated, the severity of the affliction, the manner and schedule of administration and the judgment of the prescribing physician. For example, an exemplary dosage range for oral administration is from about 5 mg to about 500 mg per day, and an exemplary intravenous administration dosage is from about 5 mg to about 500 mg per day, each depending upon the pharmacokinetics.

A daily dose is the total amount administered in a day. A daily dose may be, but is not limited to be, administered each day, every other day, each week, every 2 weeks, every month, or at a varied interval. In some embodiments, the daily dose is administered for a period ranging from a single day to the life of the subject. In some embodiments, the daily dose is administered once a day. In some embodiments, the daily dose is administered in multiple divided doses, such as in 2, 3, or 4 divided doses. In some embodiments, the daily dose is administered in 2 divided doses.

Administration of the compounds and compositions described herein can be via any accepted mode of administration for therapeutic agents including, but not limited to, oral, sublingual, subcutaneous, parenteral, intravenous, intranasal, topical, transdermal, intraperitoneal, intramuscular, intrapulmonary, vaginal, rectal, or intraocular administration. In some embodiments, the compounds and compositions described herein are administered orally or intravenously. In some embodiments, the compounds and compositions described herein is administered orally.

Pharmaceutically acceptable compositions include solid, semi-solid, liquid and aerosol dosage forms, such as tablet, capsule, powder, liquid, suspension, suppository, and aerosol forms. The compounds and compositions described herein can also be administered in sustained or controlled release dosage forms (e.g., controlled/sustained release pill, depot injection, osmotic pump, or transdermal (including electrotransport) patch forms) for prolonged timed, and/or pulsed administration at a predetermined rate. In some embodiments, the compositions are provided in unit dosage forms suitable for single administration of a precise dose.

The compounds and compositions described herein can be administered either alone or in combination with one or more conventional pharmaceutical carriers or excipients (e.g., mannitol, lactose, starch, magnesium stearate, sodium saccharine, talcum, cellulose, sodium crosscarmellose, glucose, gelatin, sucrose, magnesium carbonate). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate). Generally, depending on the intended mode of administration, the pharmaceutical composition will contain about 0.005% to 95%, or about 0.5% to 50%, by weight of a compound disclosed and/or described herein. Actual methods of preparing such dosage forms are known, or will be apparent, to those skilled in this art; for example, sec Remington's Pharmaceutical Sciences, Mack Publishing Company, Easton, Pennsylvania.

In some embodiments, the compositions will take the form of a pill or tablet and thus the composition may contain, along with the compounds described herein, one or more of a diluent (e.g., lactose, sucrose, dicalcium phosphate), a lubricant (e.g., magnesium stearate), and/or a binder (e.g., starch, gum acacia, polyvinylpyrrolidine, gelatin, cellulose, cellulose derivatives). Other solid dosage forms include a powder, marume, solution or suspension (e.g., in propylene carbonate, vegetable oils or triglycerides) encapsulated in a gelatin capsule.

Liquid pharmaceutically administrable compositions can, for example, be prepared by dissolving, dispersing or suspending etc. the compounds described herein and optional pharmaceutical additives in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of the compounds contained in such parenteral compositions depends, for example, on the physical nature of the compounds, the activity of the compounds, and the needs of the subject. However, percentages of active ingredient of 0.01% to 10% in solution are employable, and may be higher if the composition is a solid which will be subsequently diluted to another concentration. In some embodiments, the composition will comprise from about 0.2 to 2% of a compound described herein in solution.

Pharmaceutical compositions of the compounds and compositions described herein may also be administered to the respiratory tract as an aerosol or solution for a nebulizer, or as a microfine powder for insufflation, alone or in combination with an inert carrier such as lactose. In such a case, the particles of the pharmaceutical composition may have diameters of less than 50 microns, or in some embodiments, less than 10 microns.

In addition, pharmaceutical compositions can include compounds described herein and one or more additional medicinal agents, pharmaceutical agents, adjuvants, and the like.

### Kits

Also provided are articles of manufacture and kits containing any of the compounds and compositions provided herein. The article of manufacture may comprise a container with a label. Suitable containers include, for example, bottles, vials, and test tubes. The containers may be formed from a variety of materials such as glass or plastic. The container may hold a pharmaceutical composition provided herein. The label on the container may indicate that the pharmaceutical composition is used for preventing, treating or suppressing a condition described herein, and may also indicate directions for either in vivo or in vitro use.

In one aspect, provided herein are kits containing the compounds and compositions described herein and instructions for use. The kits may contain instructions for use in the treatment of any disease provided herein in a subject in need thereof. A kit may additionally contain any materials or equipment that may be used in the administration of the compounds and compositions, such as vials, syringes, or IV bags. A kit may also contain sterile packaging.

### Enumerated Embodiments

1. A compound of Formula (I):

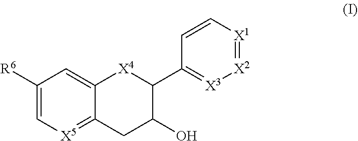

or a pharmaceutically acceptable salt, stereoisomer or tautomer thereof, wherein:
X¹ is N or CR¹;
R¹ is H, OH, halo, or C₁₋₆ alkoxy;
X² is N or CR²;
R² is H, OH, halo, C₁₋₆ alkyl optionally substituted with one or more halo, or C₁₋₆ alkoxy;
X³ is N or CR³;
R³ is H or OH;
X⁴ is O or NR⁴;
R⁴ is H or CH₃;
X⁵ is N or CR⁵;
R⁵ is H, OH, or C₁₋₆ alkoxy;
provided that if X⁴ is NR⁴, none of X¹, X², X³, and X⁵ is N, and if X⁴ is O, then one of X¹, X²,
X³, and X⁵ is N; and
R⁶ is H, OH, or C₁₋₆ alkoxy.

2. The compound of embodiment 1, wherein the compound is of Formula (Ia):

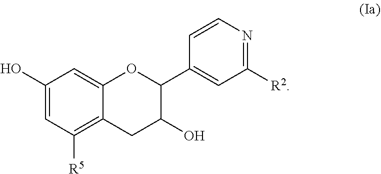

3. The compound of embodiment 2, wherein R⁵ is H or OH.

4. The compound of embodiment 2 or 3, wherein R² is H or methoxy.

5. The compound of embodiment 1, wherein the compound is of Formula (Ib):

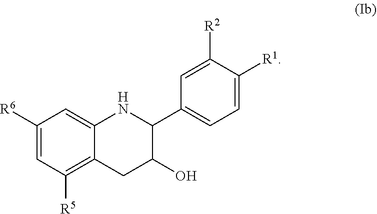

6. The compound of embodiment 5, wherein R⁵ is H or methoxy.

7. The compound of embodiment 5 or 6, wherein R⁶ is H or methoxy.

8. The compound of embodiment 1, wherein the compound is of Formula (Ic):

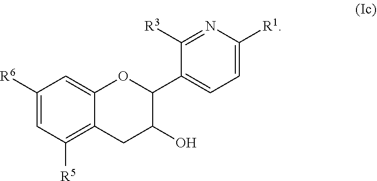

9. The compound of embodiment 8, wherein R⁵ is H or OH.

10. The compound of embodiment 8 or 9, wherein R⁶ is H or OH.

11. The compound of any one of embodiments 8-10, wherein R³ is H or OH.

12. The compound of any one of embodiments 8-11, wherein R¹ is H or methoxy.

13. The compound of embodiment 1, wherein the compound is of Formula (Id):

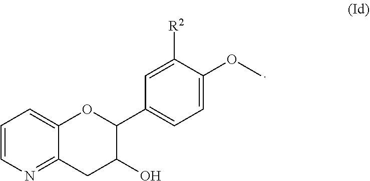

14. The compound of embodiment 13, wherein R² is F or CF₃.

15. The compound of embodiment 1, wherein the compound is of Formula (Ie):

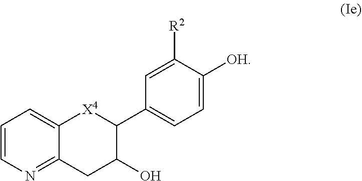

16. The compound of embodiment 15, wherein R² is H, F, or CF₃.

17. The compound of embodiment 15, wherein the compound is of Formula (If):

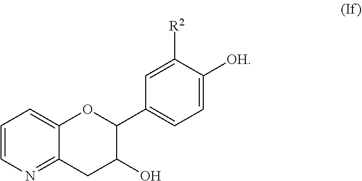

18. The compound of embodiment 17, wherein R² is H, F, or CF₃.

19. The compound of embodiment 15, wherein the compound is of Formula (Ig):

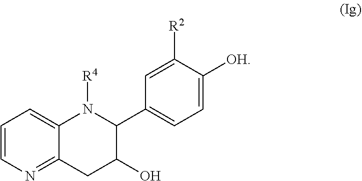

20. The compound of embodiment 19, wherein R⁴ is methyl.

21. The compound of embodiment 19 or 20, wherein R² is F or CF₃.

22. The compound of embodiment 1, wherein the compound is of Formula (Ih):

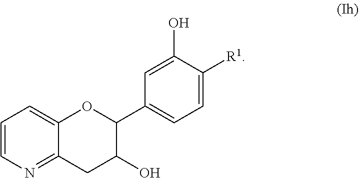

23. The compound of embodiment 22, wherein R¹ is H or F.

24. The compound of embodiment 1, wherein the compound is of Formula (Ii):

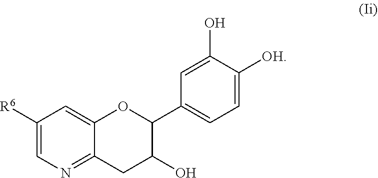

25. The compound of embodiment 24, wherein R⁶ is H or OH.

26. A compound of Formula (II):

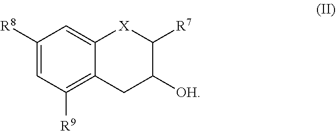

or a pharmaceutically acceptable salt, stereoisomer or tautomer thereof, wherein:
X is O or NCH₃;
R⁷ is C₁₋₆ alkyl, C₃₋₁₀ cycloalkyl optionally substituted with one or more OH or C₁₋₆ alkoxy, or 5-6 membered heterocyclyl optionally substituted with one or more C₁₋₆ alkyl; and
R⁸ and R⁹ are each independently H or OH.

27. The compound of embodiment 26, wherein the compound is of Formula (IIa):

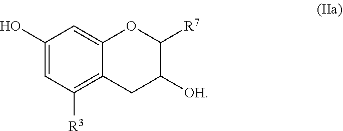

28. The compound of embodiment 27, wherein the compound is of Formula (IIb):

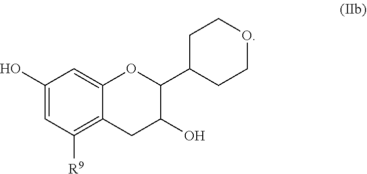

29. The compound of embodiment 28, wherein R⁹ is H.

30. The compound of embodiment 28, wherein R⁹ is OH.

31. The compound of embodiment 26, wherein the compound is of Formula (IIc):

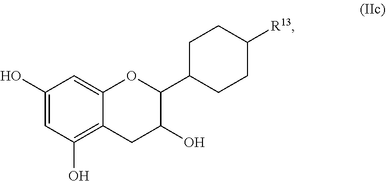

wherein R¹³ is H or OR¹⁴; and
R¹⁴ is H or methyl.

32. The compound of embodiment 31, wherein R¹³ is H.

33. The compound of embodiment 31, wherein R¹³ is OR¹⁴, and R¹⁴ is H.

34. The compound of embodiment 31, wherein R¹³ is OR¹⁴, and R¹⁴ is methyl.

35. A compound of Formula (III):

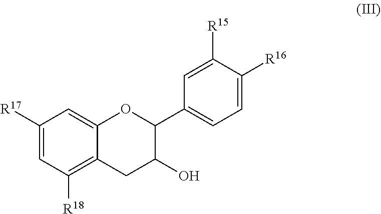

or a pharmaceutically acceptable salt, stereoisomer or tautomer thereof, wherein:
R¹⁵ is H, C₁₋₆ alkoxy, halo, or C₁₋₆ alkyl optionally substituted with halo;
R¹⁶ is H, halo, or OH; and
R¹⁷ and R¹⁸ are each independently H or OH.

36. The compound of embodiment 35, wherein R¹⁵ is OH.

37. The compound of embodiment 35, wherein R¹⁵ is methoxy.

38. The compound of embodiment 35, wherein R¹⁵ is CF₃.

39. The compound of any one of embodiments 35-39, wherein R¹⁶ is OH.

40. The compound of any one of embodiments 35-39, wherein R¹⁶ is F.

41. The compound of any one of embodiments 35-41, wherein R¹⁷ is OH.

42. The compound of any one of embodiments 35-41, wherein R¹⁸ is H.

43. A compound selected from the group consisting of:

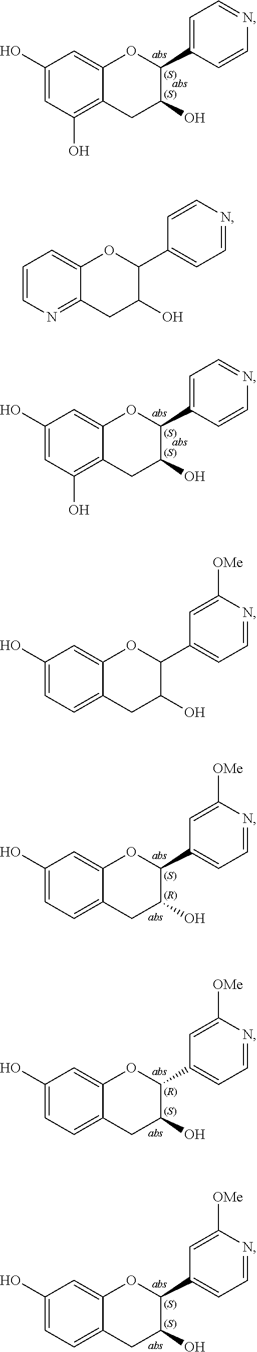

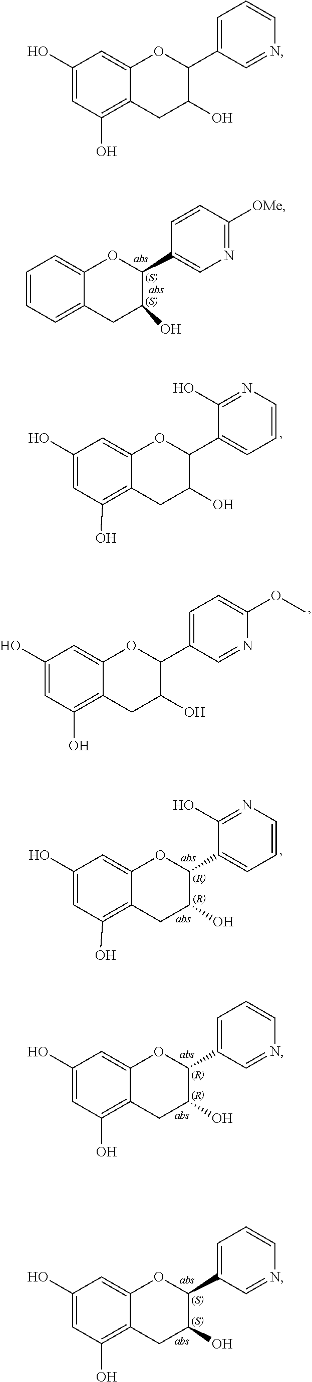

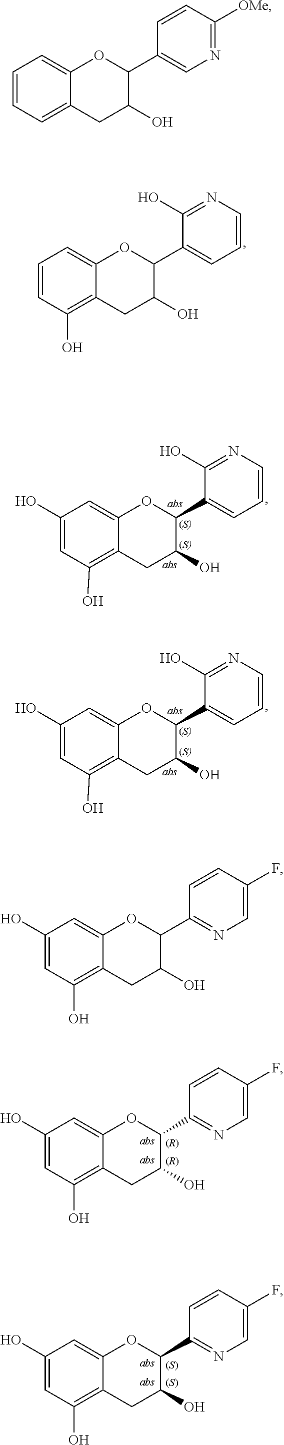

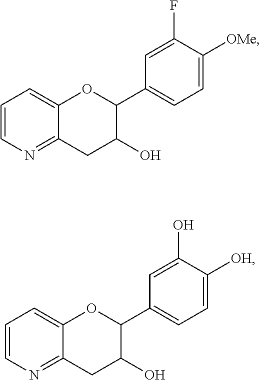 [SMILES: COc1ccc(C2Oc3cccnc3CC2O)cc1F ; Oc1ccc(C2Oc3cccnc3CC2O)cc1O]

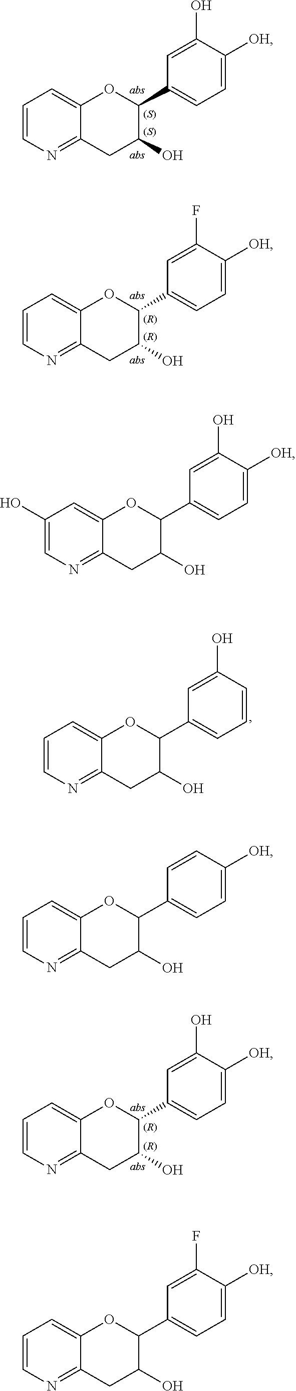

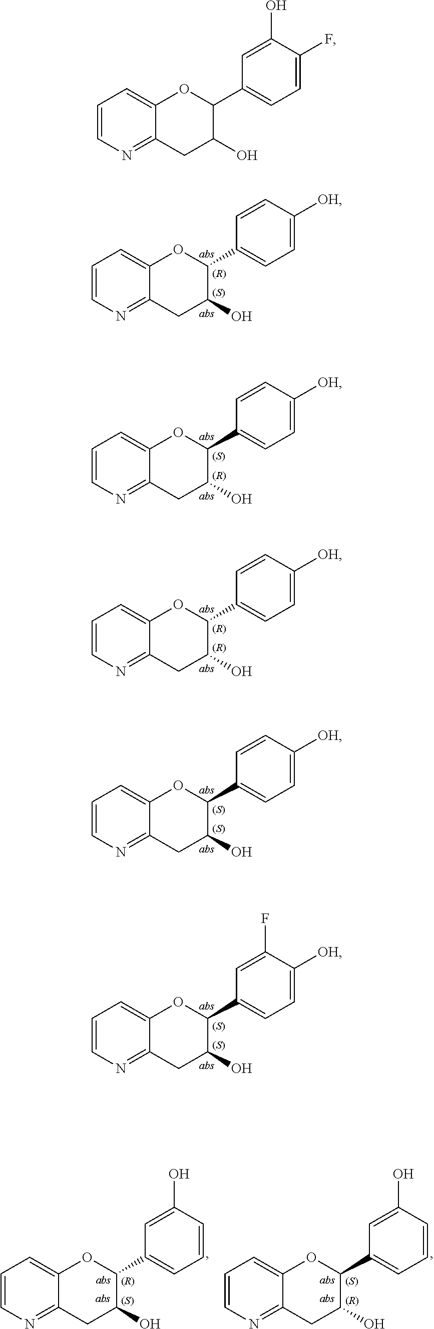

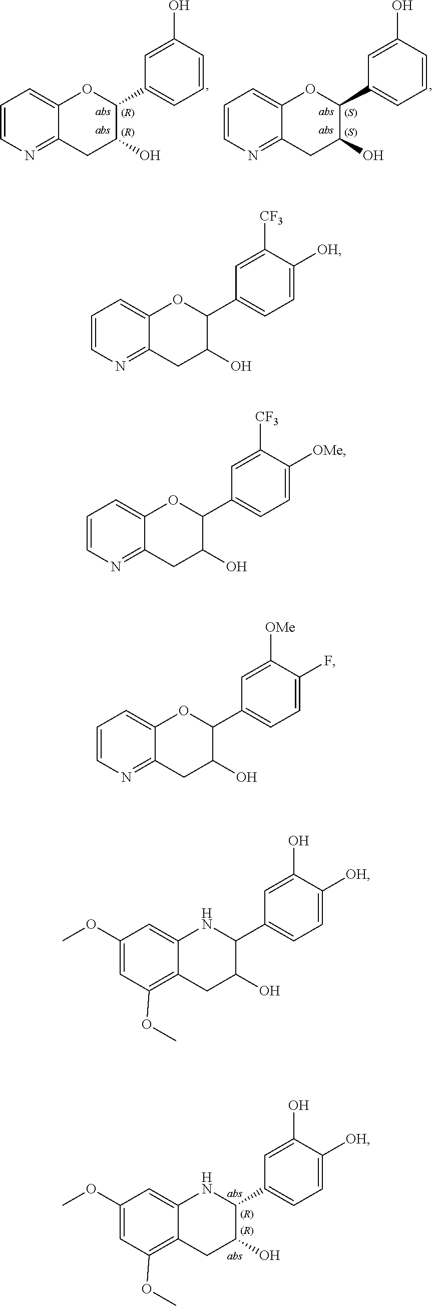

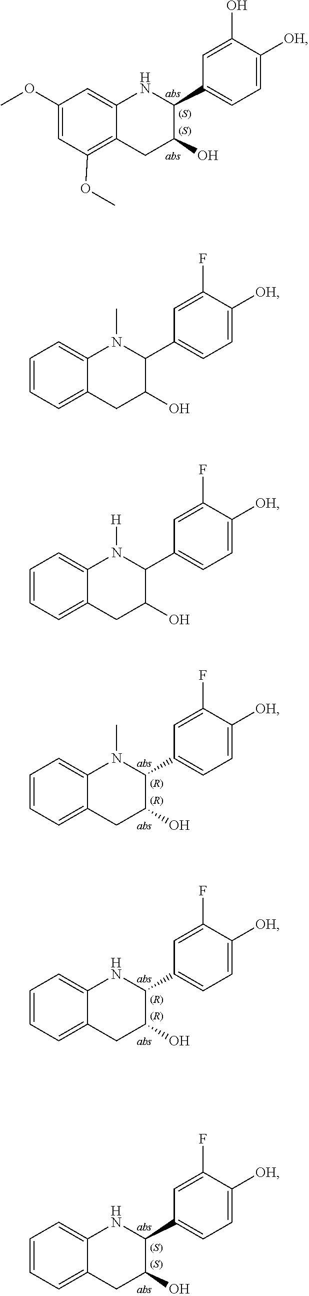

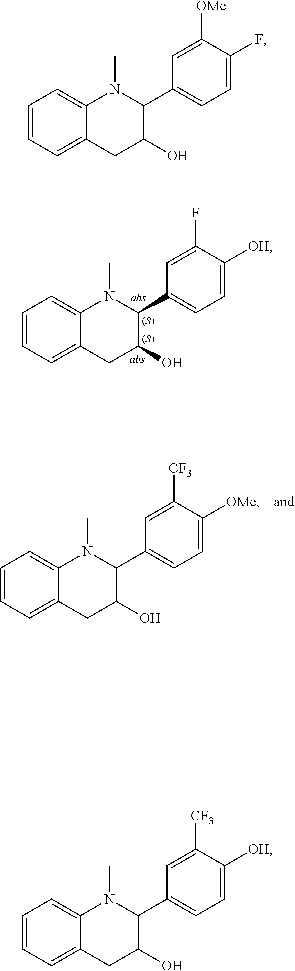

or a pharmaceutically acceptable salt, stereoisomer or tautomer thereof.

44. A compound selected from the group consisting of:

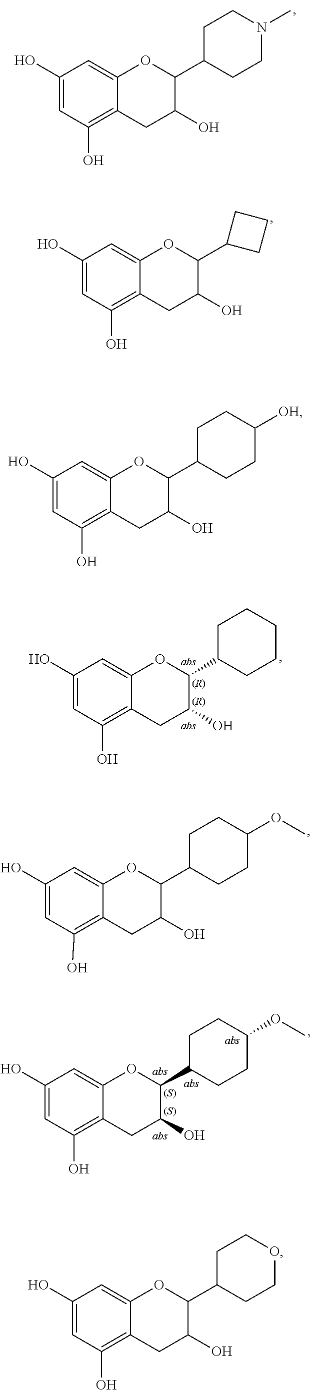

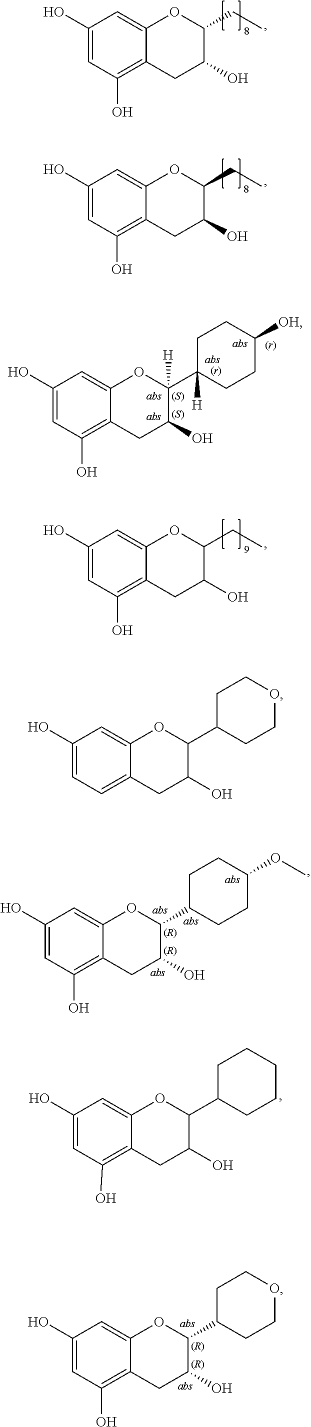

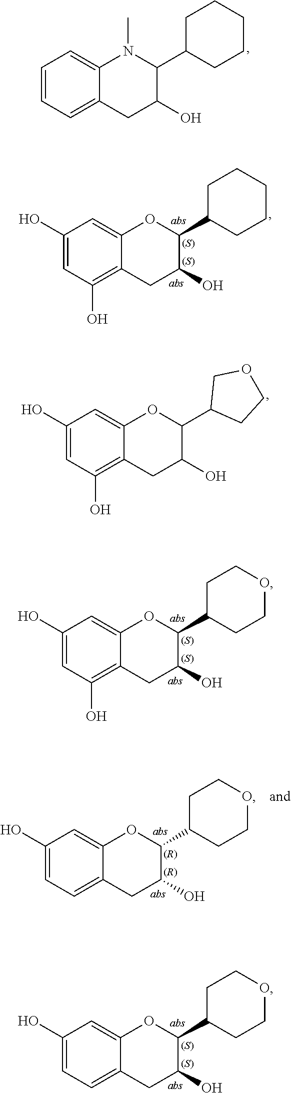

or a pharmaceutically acceptable salt, stereoisomer or tautomer thereof.

45. A compound selected from the group consisting of:

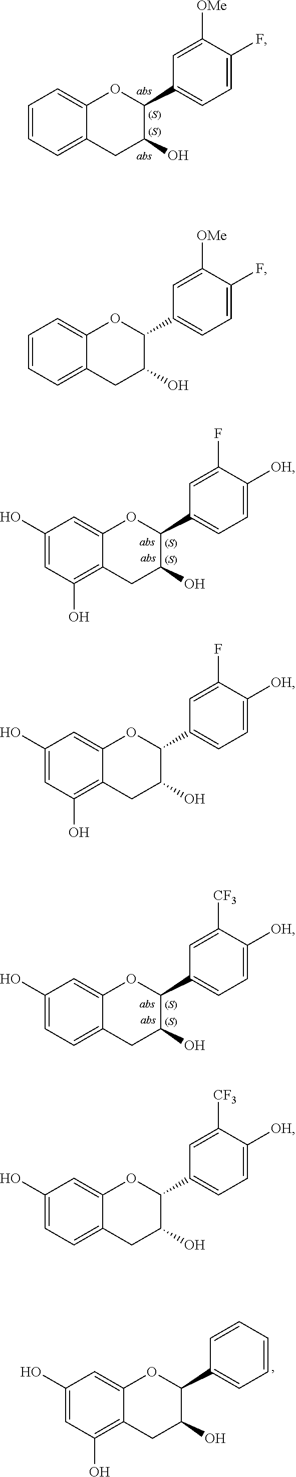

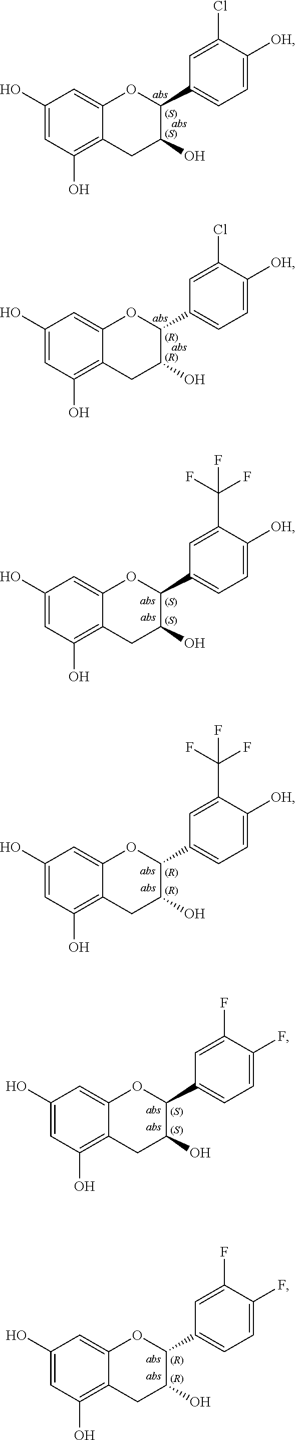

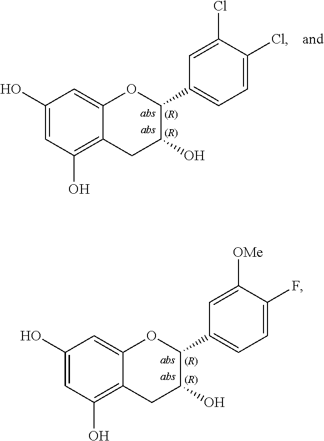

or a pharmaceutically acceptable salt, stereoisomer or tautomer thereof.

46. A compound selected from the group consisting of

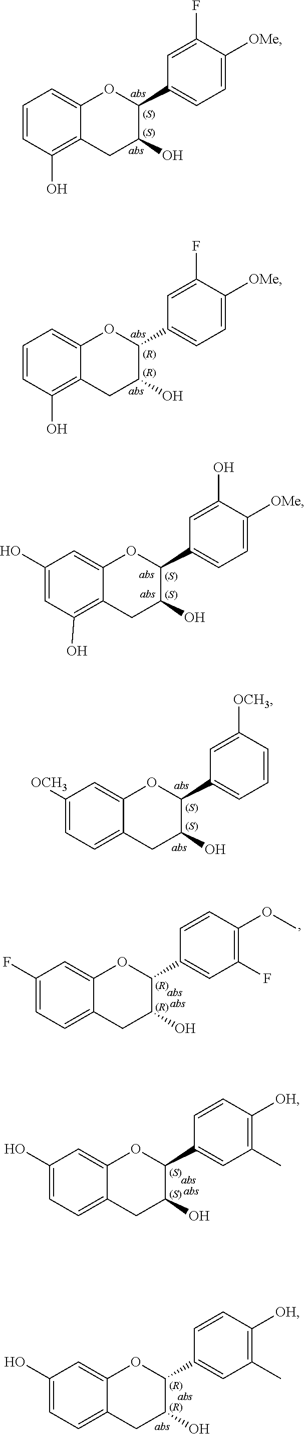

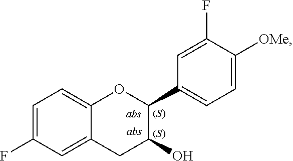

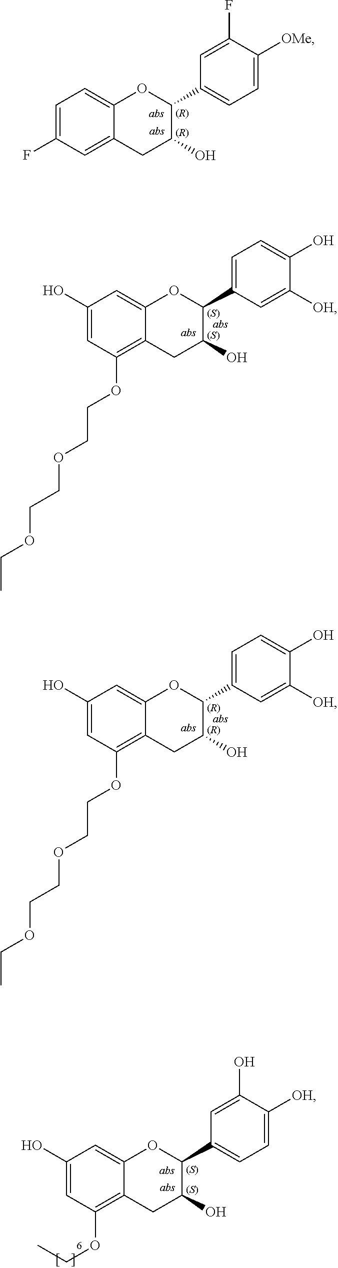

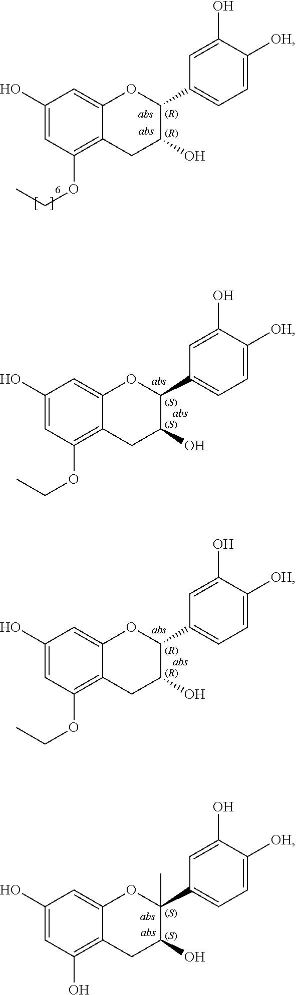

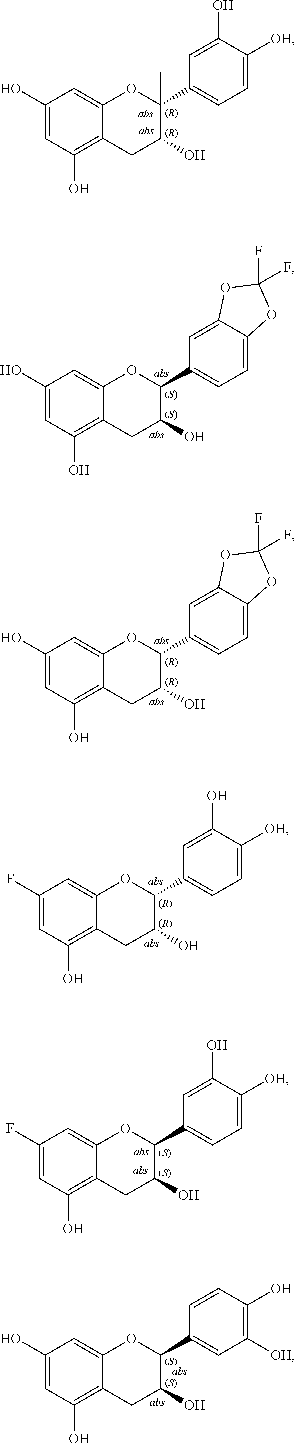

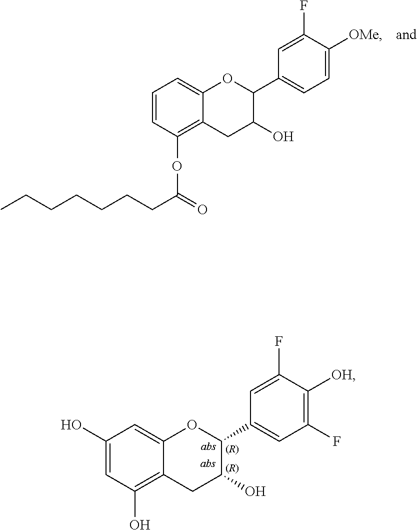

or a pharmaceutically acceptable salt, stereoisomer or tautomer thereof.

47. A pharmaceutical composition comprising a compound of any one of embodiments 1-46, a pharmaceutically acceptable salt thereof, and a pharmaceutically acceptable carrier.

48. A compound, or a pharmaceutically acceptable salt thereof, of any one of embodiments 1-46 for use as therapeutically active substance.

49. Use of a compound, or a pharmaceutically acceptable salt thereof, of any one of embodiments 1-46 or a pharmaceutical composition of embodiment 47 for the preparation of a medicament for the treatment of a disease, disorder or condition.

Certain specific aspects and embodiments will be explained in more detail with reference to the following examples, which are provided only for purposes of illustration and should not be construed as limiting the scope in any manner.

### Examples

### List of Abbreviations

AICAR 5-aminoimidazole-4-carboxamide-1-β-D-ribofuranoside
AMPK AMP activated protein kinase
BLQ Below the limit of quantitation
BSA Bovine Serum Albumin
CMC Carboxy methyl Cellulose
DMEM Dulbecco's Modified Eagle Medium
ELISA Enzyme-linked immunosorbent assay
ESI-MS Electrospray ionization mass spectrometry
FBS Fetal bovine serum
HPLC High performance liquid chromatography
HRP Horseradish peroxidase
IP Intra-peritoneal
IVC Individually ventilated caging
LCMS Liquid chromatography-mass spectrometry
LPS Lipopolysaccharide
NMP N-methyl-2-pyrrolidone
PBS Phosphate buffered saline
PGC1α PPARG coactivator 1 alpha
PO Per Oral
PPARGC1A Peroxisome proliferator-activated receptor gamma coactivator 1-alpha
RIPA Radioimmunoprecipitation assay
RPM Rotation per minute
RT Room temperature
TMB Tumor mutational burden
μl Micro litre
° C. Degree Celsius
BID Twice a day dosing

### Example 1: Synthesis of Compound 75

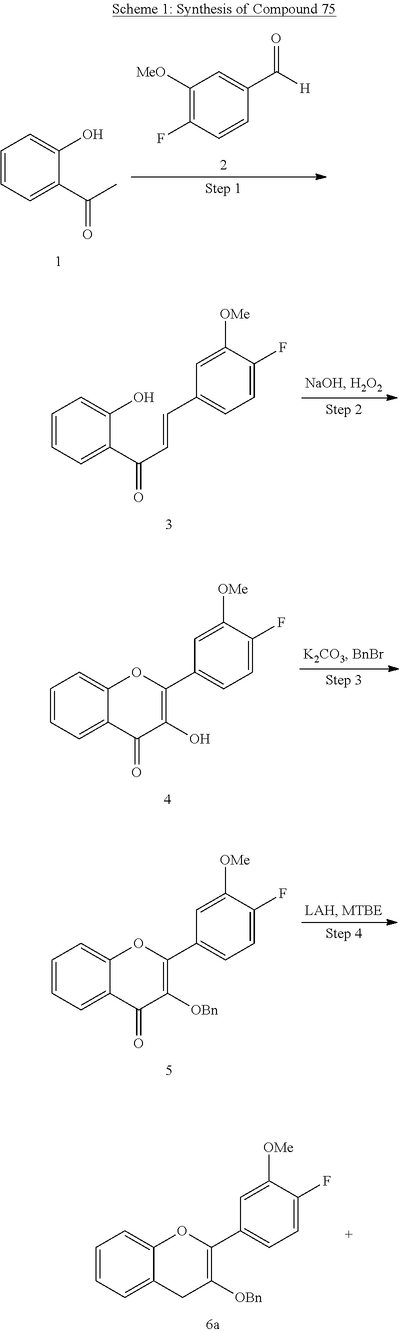

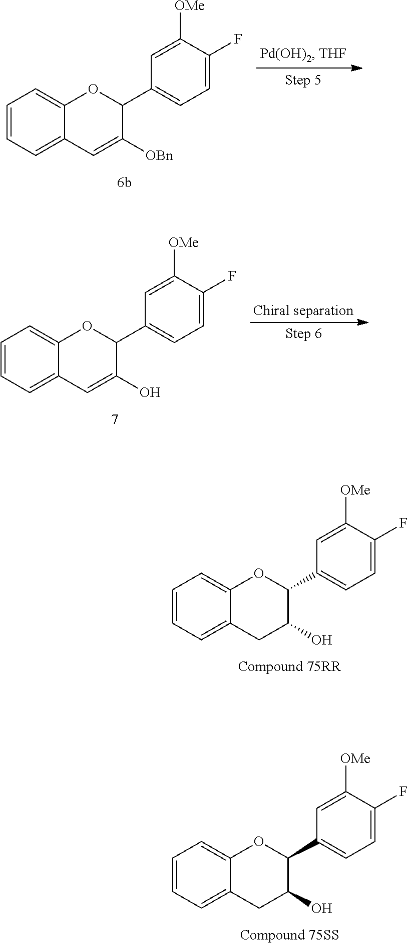 [SMILES: COc1cc(C2Oc3ccccc3C=C2OCc2ccccc2)ccc1F ; COc1cc(C2Oc3ccccc3C=C2O)ccc1F ; COc1cc([C@H]2Oc3ccccc3C[C@H]2O)ccc1F ; COc1cc([C@@H]2Oc3ccccc3C[C@@H]2O)ccc1F]

### 1. Synthesis of (E)-3-(4-fluoro-3-methoxyphenyl)-1-(2-hydroxyphenyl)prop-2-en-1-one (3)

To a stirred solution of 1-(2-hydroxyphenyl)ethan-1-one (1, 5.2 g, 38.23 mmol, 1.0 eq) and 4-fluoro-3-methoxybenzaldehyde (2, 5.9 g, 38.23 mmol, 1.0 eq) in ethanol (100 mL), 50% sodium hydroxide solution (15 mL) was added dropwise and stirred at room temperature for overnight. After completion of the reaction. The reaction mixture was poured into ice water and neutralized with 1N HCl solution, the precipitate was filtered and dried to obtain (E)-3-(4-fluoro-3-methoxyphenyl)-1-(2-hydroxyphenyl)prop-2-en-1-one (3) as a yellow solid (5.0 g, 48.07%). ESI-MS: calculated m/z 272.28 [M]+, observed m/z 273.0 [M+H]+.

### 2. Synthesis of 2-(4-fluoro-3-methoxyphenyl)-3-hydroxy-4H-chromen-4-one (4)

To a stirred solution of (E)-3-(4-fluoro-3-methoxyphenyl)-1-(2-hydroxyphenyl)prop-2-en-1-one (3, 5.0 g, 18.38 mmol) and 20% sodium hydroxide solution (10 mL) in methanol (80 mL), 30% H₂O₂ solution (10 mL) was added dropwise at 0° C. and stirred at room temperature for 2.5 h. After completion of the reaction, the reaction mixture was acidified with a cold 1N HCl solution. The yellow precipitate formed was filtered and dried to obtain 2-(4-fluoro-3-methoxyphenyl)-3-hydroxy-4H-chromen-4-one (4) as a yellow solid (4.3 g, 81.09%). ESI-MS: calculated m/z 286.26 [M]⁺, observed m/z 286.9 [M+H]⁺.

### 3. Synthesis of 3-(benzyloxy)-2-(4-fluoro-3-methoxyphenyl)-4H-chromen-4-one (5)

To a stirred solution of 2-(4-fluoro-3-methoxyphenyl)-3-hydroxy-4H-chromen-4-one (4, 4.3 g, 15.03 mmol, 1.0 eq) and K₂CO₃ (4.14 g, 30.06 mmol, 2.0 eq) in N,N-dimethylformamide (50 mL), benzyl bromide (2.75 mL, 22.55 mmol, 1.5 eq) was added and stirred at room temperature for 16 h. After completion of the reaction, the reaction was diluted with ice cool water. The crude product was filtered and dried to obtain 3-(benzyloxy)-2-(4-fluoro-3-methoxyphenyl)-4H-chromen-4-one (5) as an off-white solid (2.3 g, 40.7%). ESI-MS: calculated m/z 376.38 [M]⁺, observed m/z 376.9 [M+H]⁺.

### 4. Synthesis of 3-(benzyloxy)-2-(4-fluoro-3-methoxyphenyl)-4H-chromene (6a) and 3-(benzyloxy)-2-(4-fluoro-3-methoxyphenyl)-2H-chromene (6b)

To a stirred solution of 3-(benzyloxy)-2-(4-fluoro-3-methoxyphenyl)-4H-chromen-4-one 5 (2.3 g, 6.11 mmol, 1.0 eq) in methyl tertiary-butyl ether (50 mL), lithium aluminium hydride (0.93 g, 24.46 mmol, 4.0 eq) was added at 40° C. and stirred at 90° C. for 2.5 h. After completion of the reaction, the reaction mixture was cooled and quenched with ice water (30 mL), and extracted with ethyl acetate (3×100 mL). The combined organic layers were dried over anhydrous sodium sulphate and concentrated to obtain the crude product. The crude product was purified by column chromatography (100-200 silica mesh) using 5% ethyl acetate in hexane to obtain a mixture of 3-(benzyloxy)-2-(4-fluoro-3-methoxyphenyl)-4H-chromene (6a) and 3-(benzyloxy)-2-(4-fluoro-3-methoxyphenyl)-2H-chromene (6b) as yellow sticky material. ESI-MS: calculated m/z 362.4 [M]⁺, observed m/z 362.9 [M+1]⁺.

### 5. Synthesis of (2S,3S)-2-(4-fluoro-3-methoxyphenyl)chroman-3-ol (Compound 75)

To a stirred solution of 3-(benzyloxy)-2-(4-fluoro-3-methoxyphenyl)-4H-chromene 6a and 3-(benzyloxy)-2-(4-fluoro-3-methoxyphenyl)-2H-chromene 6b (1.8 g, 4.96 mmol, 1.0 eq) in THF (30 mL), Pd(OH)₂ (1.8 g) was added at room temperature. The suspension was hydrogenated in a parr shaker pressure under 60 psi of hydrogen pressure for 3 h. The reaction was filtered over celite and concentrated to afford the crude product. The obtained crude was purified by column chromatography (100-200 silica mesh), to afford racemic 2-(4-fluoro-3-methoxyphenyl)chroman-3-ol (7) as a white solid (950 mg, 69.85%). The enantiomers were separated by chiral preparative HPLC [Chiralpak AD H (250×10) mm]. Peak-1 2-(4-fluoro-3-methoxyphenyl)chroman-3-ol, Compound 75a): 46.0 mg; HPLC: 99.12%; Chiral RT: 11.15 (ee=100%). 1H NMR (300 MHz, DMSO-d6): δ/ppm 7.28-7.10 (m, 4H), 6.85-6.83 (m, 3H), 5.05 (s, 1H), 4.93-4.92 (d, J=4.5 Hz, 1H), 4.14 (bs, 1H), 3.83 (s, 3H), 3.20-3.15 (d, J=15 Hz, 1H), 2.74-2.68 (d, J=18 Hz, 1H). ESI-MS: calculated m/z 274.29 [M]⁺, observed m/z 272.9 [M−H]⁻. Peak 2 2-(4-fluoro-3-methoxyphenyl)chroman-3-ol, Compound 75b): 37.8 mg; HPLC: 99.9%; Chiral RT: 12.91 (ee=99.71%). 1H NMR (300 MHz, DMSO-d6): δ/ppm 7.28-7.04 (m, 5H), 6.85-6.82 (m, 2H), 5.06 (s, 1H), 4.93-4.92 (d, J=4.6 Hz, 1H), 4.15 (bs, 1H), 3.83 (s, 3H), 3.21-3.16 (d, J=15 Hz, 1H), 2.74-2.69 (d, J=15 Hz, 1H). ESI MS: calculated m/z 274.29 [M]⁺, observed m/z 272.9 [M−H]⁻.

### Example 2: Synthesis of Compound 76

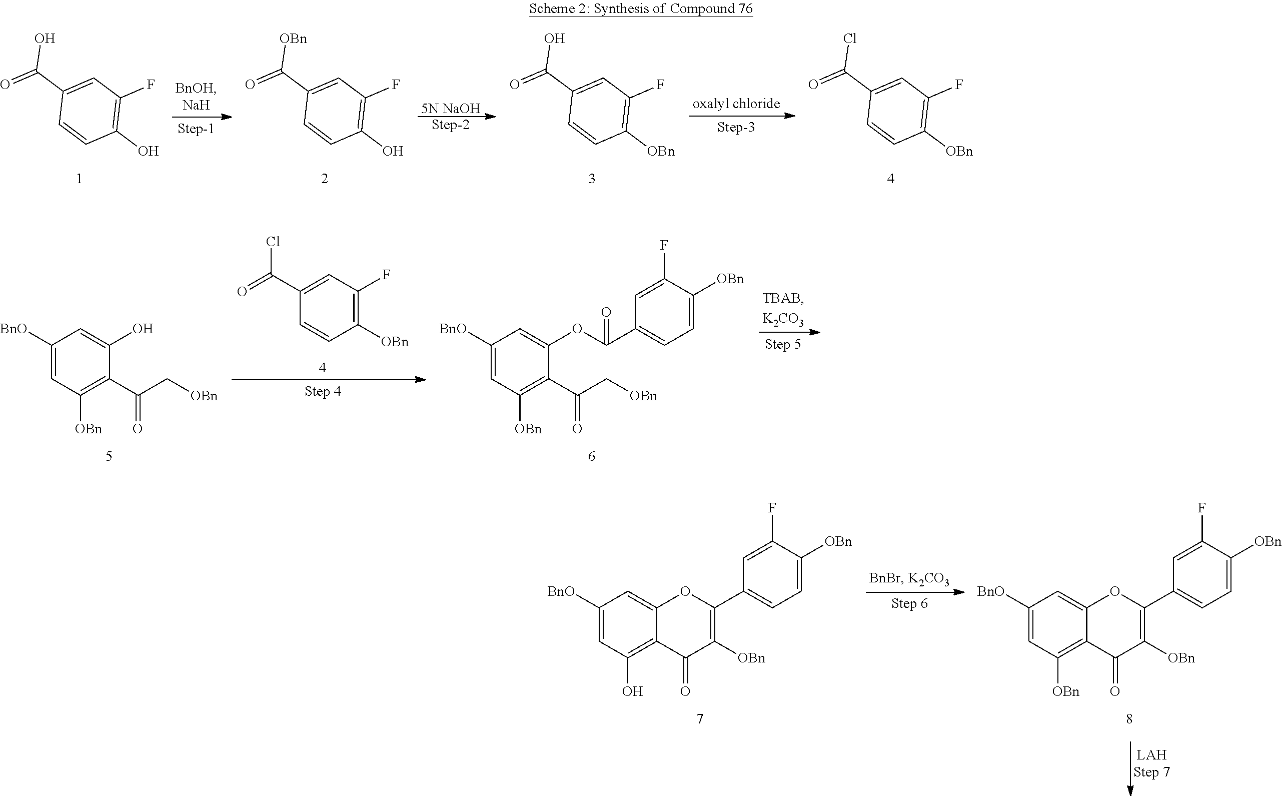

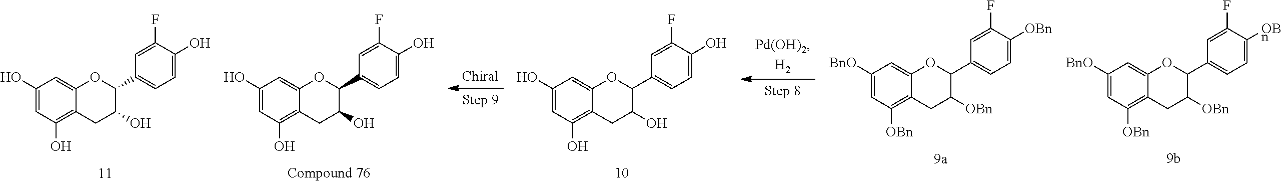 [SMILES: Oc1cc(O)c2c(c1)O[C@H](c1ccc(O)c(F)c1)[C@H](O)C2 ; Oc1cc(O)c2c(c1)O[C@@H](c1ccc(O)c(F)c1)[C@@H](O)C2 ; Oc1cc(O)c2c(c1)OC(c1ccc(O)c(F)c1)C(O)C2 ; Fc1cc(C2Oc3cc(OCc4ccccc4)cc(OCc4ccccc4)c3CC2OCc2ccccc2)ccc1OCc1ccccc1 ; Fc1cc(C2Oc3cc(OCc4ccccc4)cc(OCc4ccccc4)c3CC2OCc2ccccc2)ccc1OCc1ccccc1]

### 1. Synthesis of benzyl 4-(benzyloxy)-3-fluorobenzoate (2)

To a stirred solution of 3-fluoro-4-hydroxybenzoic acid (1, 20.00 g, 128 mmol, 1.0 eq) in N,N-dimethylformamide (80 mL), benzyl bromide (38 ml, 320 mmol, 2.5 eq) and potassium carbonate (53 g, 384 mmol, 3.0 eq) were added and stirred at room temperature for 16 hrs. After completion of the reaction, the reaction mixture was quenched with ice water (150 mL), and the precipitate obtained was collected and washed with diethyl ether and n-pentane to obtain benzyl 4-(benzyloxy)-3-fluorobenzoate (2), as a light brown solid (18.0 g, 42%). ESI-MS: calculated m/z 336.3 [M]+, observed m/z 359 [M+Na]+.

### 2. Synthesis of 4-(benzyloxy)-3-fluorobenzoic acid (3)

Why To a stirred solution of benzyl 4-(benzyloxy)-3-fluorobenzoate (2, 18.0 g, 32.1 mmol, 1.0 eq) in a mixture of methanol (20 mL) and THF (20 mL), 20% sodium hydroxide solution (20 mL) was added at room temperature and stirred at 60° C. for 3 h. After completion of the reaction, the solvent was evaporated under reduced pressure and mixture was neutralized with 2N hydrochloric acid (pH≈6-7). Solid filtered and washed with water and diethyl ether and dried under vacuum to afford 4-(benzyloxy)-3-fluorobenzoic acid (3) as white solid (10 g, 76%). ESI-MS: calculated m/z 246 [M]+, observed m/z 247 [M+H]⁺.

### 3. Synthesis of 4-(benzyloxy)-3-fluorobenzoyl chloride (4)

To a stirred solution of 4-(benzyloxy)-3-fluorobenzoic acid (3, 10 g, 40.65 mmol, 1.0 eq) in dichloromethane (50 mL), oxalyl chloride (21 mL, 243 mmol, 6.0 eq) was added, followed by dropwise addition of N,N′-dimethylformamide (1.0 mL, 72.4 mmol, 0.25 eq) at 0° C. The reaction mixture was allowed to stir at room temperature for 16 h. After completion of the reaction, the solvent was evaporated under reduced pressure to afford 4-(benzyloxy)-3-fluorobenzoyl chloride (4) as a yellow solid (8.5 g, 74.5% yield).

### 4. Synthesis of 3,5-bis(benzyloxy)-2-(2-(benzyloxy)acetyl)phenyl 4-(benzyloxy)-3-fluorobenzoate (6)

To a stirred solution of 2-(benzyloxy)-1-(2,4-bis(benzyloxy)-6-hydroxyphenyl)ethan-1-one (5, 10.00 g, 22.02 mmol, 1.0 eq), 4-dimethylaminopyridine (0.13 g, 1.101 mmol, 0.05 eq) and triethylamine (14.3 mL, 110.1 mmol, 5.0 eq) in dry dichloromethane (60.0 mL), 4-(benzyloxy)-3-fluorobenzoyl chloride (4, 6.5 g, 26.42 mmol, 1.2 eq) was added portion-wise and stirred at room temperature for 3 h. After completion of the reaction, the reaction mixture was diluted with water (200 mL) extracted with ethyl acetate (250 mL), dried over anhydrous sodium sulphate, and concentrated. The crude product was purified by column chromatography (100-200 silica mesh), using 30% ethyl acetate in hexane to obtain 3,5-bis(benzyloxy)-2-(2-(benzyloxy)acetyl)phenyl 4-(benzyloxy)-3-fluorobenzoate (6) as a white solid (8.0 g, 53%).

### 5. Synthesis of 3,7-bis(benzyloxy)-2-(4-(benzyloxy)-3-fluorophenyl)-5-hydroxy-4H-chromen-4-one (7)

To a stirred solution of 3,5-bis(benzyloxy)-2-(2-(benzyloxy)acetyl)phenyl 4-(benzyloxy)-3-fluorobenzoate (6, 15.0 g, 21.9 mmol, 1.0 eq) in toluene (60.0 mL), tetrabutylammonium bromide (14.1 g, 43.9 mmol, 2.0 eq) and potassium carbonate (15.2 g, 109.9 mmol, 5.0 eq) were added, and stirred at 100° C. for 4 hrs. After completion of the reaction, the toluene was evaporated and extracted with DCM (100 mL), and dried over anhydrous sodium sulphate. The organic layer was concentrated to obtain the crude. The crude product was purified by column chromatography (100-200 silica mesh) using 30% ethyl acetate in hexane to obtain 3,7-bis(benzyloxy)-2-(4-(benzyloxy)-3-fluorophenyl)-5-hydroxy-4H-chromen-4-one (7), as an off-white solid (10 g, 79.2%).

### 6. Synthesis of 3,5,7-tris(benzyloxy)-2-(4-(benzyloxy)-3-fluorophenyl)-4H-chromen-4-one (8)

To a stirred solution of 3,7-bis(benzyloxy)-2-(4-(benzyloxy)-3-fluorophenyl)-5-hydroxy-4H-chromen-4-one (7, 10.0 g, 17.4 mmol, 1.0 eq) in N,N′-dimethylformamide (40.0 mL), benzyl bromide (3.57 g, 20.9 mmol, 1.2 eq) and potassium carbonate (3.60 g, 26.1 mmol, 1.5 eq) were added, and stirred at room temperature for 16 hrs. After completion of the reaction, the reaction mixture was diluted with ice water (100 mL), and the precipitated solid was filtered and washed with diethyl ether and n-pentane to afford 3,5,7-tris(benzyloxy)-2-(4-(benzyloxy)-3-fluorophenyl)-4H-chromen-4-one (8), as off white solid (7.0 g, 60.5%).

### 7. Synthesis of 3,5,7-tris(benzyloxy)-2-(4-(benzyloxy)-3-fluorophenyl)-4H-chromene (9a) & 3,5,7-tris(benzyloxy)-2-(4-(benzyloxy)-3-fluorophenyl)-2H-chromene (9b)

To a stirred solution of 3,5,7-tris(benzyloxy)-2-(4-(benzyloxy)-3-fluorophenyl)-4H-chromen-4-one (8, 5.00 g, 7.5 mmol, 1.0 eq) in methyl tertiary-butyl ether (50.0 mL), lithium aluminium hydride (1.13 g, 30 mmol, 4.0 eq) was added at 50° C., after addition, the reaction mixture was stirred at 80° C. for 3 hrs. After completion of the reaction, the reaction mixture was quenched with ice water (50 mL), extracted with ethyl acetate (200 mL), washed with brine, and dried over anhydrous sodium sulphate. The organic layer was concentrated to obtain the crude. The crude product was purified by column chromatography (100-200 silica mesh) using 20% ethyl acetate in hexane to obtain mixture of 3,5,7-tris(benzyloxy)-2-(4-(benzyloxy)-3-fluorophenyl)-4H-chromene (9a) and 3,5,7-tris(benzyloxy)-2-(4-(benzyloxy)-3-fluorophenyl)-2H-chromene (9b), as a brown oil (2.00 g, 41%). ESI-MS: calculated m/z 650 [M]+, observed m/z 650 [M+H]+.

### 8. Synthesis of 2-(3-fluoro-4-hydroxyphenyl)chromane-3,5,7-triol (Compound 76)

To a stirred solution of 3,5,7-tris(benzyloxy)-2-(4-(benzyloxy)-3-fluorophenyl)-4H-chromene (9a) and 3,5,7-tris(benzyloxy)-2-(4-(benzyloxy)-3-fluorophenyl)-2H-chromene (9b) (1.0 g, 1.5 mmol, 1.0 eq) in dry tetrahydrofuran (15 ml), Pd(OH)₂ (1.2 g) was added at room temperature. The suspension was hydrogenated in a parrshaker pressure 60 psi of hydrogen pressure for 3 h. The reaction was filtered over celite and concentrated to afford the crude product. The obtained crude was purified by column chromatography (100-200 silica mesh) using 20% ethyl acetate in hexane to afford 2-(3-fluoro-4-hydroxyphenyl)chromane-3,5,7-triol (10) (200 mg, 44.5%). ESI-MS: calculated m/z 292.01 [M]+, observed m/z 293.01 [M+H]+. HPLC retention time (RT): 12.99 The enantiomers were separated by chiral preparative HPLC [Chiralpak ADH (250×10) mm]. Peak-1 2-(3-fluoro-4-hydroxyphenyl)chromane-3,5,7-triol (Compound 76a): 43.8 mg; Chiral Rt 12.99 (ee>100%); HPLC 98.8%; 1H NMR (300 MHz, DMSO-d6): δ/ppm 9.75 (s, 1H), 9.17 (s, 1H), 8.94 (s, 1H), 7.19-7.15 (d, J=12.6 Hz, 1H), 7.04-7.01 (d, J=8.4 Hz, 1H), 6.89-6.86 (m, 1H), 5.90-5.89 (d, J=2.1 Hz, 1H), 5.73-5.72 (d, J=1.8 Hz, 1H), 4.82 (s, 1H), 4.79-4.78 (d, J=3 Hz, 2H), 4.02 (bs, 1H), 2.72 (m, 1H), 2.27 (m, 1H); MS (ESI) m/z: 292.01 [M+1] 293.21. Peak-2 2-(3-fluoro-4-hydroxyphenyl)chromane-3,5,7-triol (Compound 76b)): 44 mg; chiral Rt 20.65 (ee>99.9%); HPLC 99.16%; 1H NMR (300 MHz, DMSO-d6): δ/ppm 9.75 (s, 1H), 9.16 (s, 1H), 8.94 (s, 1H), 7.19-7.15 (d, J=12.6 Hz, 1H), 7.04-7.01 (d, J=8.4 Hz, 1H), 6.92-6.86 (m, 1H), 5.90-5.89 (d, J=2.1 Hz, 1H), 5.73-5.72 (d, J=1.8 Hz, 1H), 4.82 (s, 1H), 4.79-4.78 (d, J=3 Hz, 2H), 4.03 (bs, 1H), 2.72 (m, 1H), 2.27 (m, 1H); MS (ESI) m/z: 292.11 [M+1]+ 293.21.

### Example 3: Synthesis of Compound 77

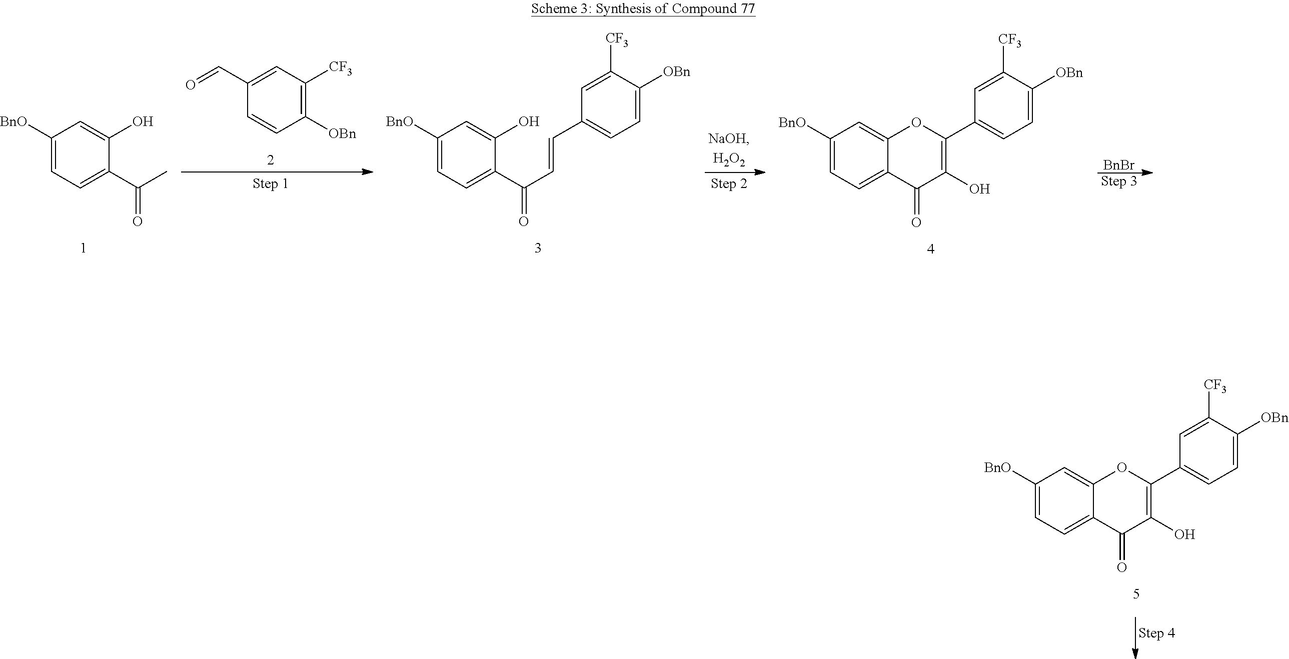

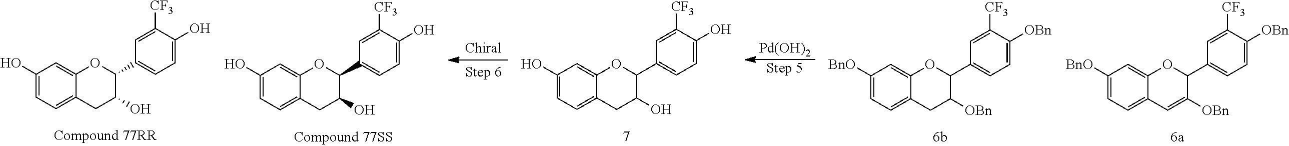

### 1. Synthesis of (E)-1-(4-(benzyloxy)-2-hydroxyphenyl)-3-(4-(benzyloxy)-3-(trifluoromethyl)phenyl)prop-2-en-1-one (3)

To a stirred solution of 1-(4-(benzyloxy)-2-hydroxyphenyl)ethan-1-one (1, 5.5 g, 22.7 mmol, 1.0 eq), in ethanol (250 mL), 50% sodium hydroxide solution (14 mL) was added and stir for 10 minute. 4-(benzyloxy)-3-(trifluoromethyl)benzaldehyde (2, 6.9 g, 24.9 mmol, 1.1 eq) was added portion-wise at room temperature and stirred at room temperature for 16 h. After completion of the reaction, ethanol was evaporated under reduced pressure, and the mixture was neutralized with 2N hydrochloric acid (pH≈6-7). Solid was filtered and washed with water and diethyl ether and dried under vacuum to afford (E)-1-(4-(benzyloxy)-2-hydroxyphenyl)-3-(4-(benzyloxy)-3-(trifluoromethyl)phenyl)prop-2-en-1-one (3) as a yellow solid (5.0 g, 45%). ESI-MS: calculated m/z 504.05 [M]+, observed m/z 505.3 [M+H]+.

### 2. Synthesis of 7-(benzyloxy)-2-(4-(benzyloxy)-3-(trifluoromethyl)phenyl)-3-hydroxy-4H-chromen-4-one (4)

To a stirred solution of (E)-1-(4-(benzyloxy)-2-hydroxyphenyl)-3-(4-(benzyloxy)-3-(trifluoromethyl)phenyl)prop-2-en-1-one (3, 5.0 g, 9.9 mmol, 1.0 eq) in methanol (150.0 mL), 20% sodium hydroxide solution (15 mL, 49.6 mmol, 5 eq) was added at 0° C., followed by dropwise addition of 30% hydrogen peroxide (8 ml, 29.7 mmol, 6.0 eq) at 0° C., mixture was stir at room temperature for 1 h. After completion of the reaction, the reaction mixture was concentrated and neutralized with 1N hydrochloric acid. Solid was filtered and washed with diethyl ether and dried under vacuum to afford 7-(benzyloxy)-2-(4-(benzyloxy)-3-(trifluoromethyl)phenyl)-3-hydroxy-4H-chromen-4-one (4), as a yellow liquid (5.0 g, 98%). ESI-MS: calculated m/z 518.2 [M]+, observed m/z 319.21 [M+H]+.

### 3. Synthesis of 3,7-bis(benzyloxy)-2-(4-(benzyloxy)-3-(trifluoromethyl)phenyl)-4H-chromen-4-one (5)

To a stirred solution of 7-(benzyloxy)-2-(4-(benzyloxy)-3-(trifluoromethyl)phenyl)-3-hydroxy-4H-chromen-4-one (4, 5.0 g, 9.65 mmol, 1.0 eq) in N,N-dimethylformamide (20 ml), anhydrous potassium carbonate (2.6 g, 19.3 mmol, 2.0 eq) and benzyl bromide (1.7 mL, 14.4 mmol, 1.5 eq) were added at room temperature. The reaction mixture was stirred at room temperature for 12 h. After completion of the reaction, the reaction mixture was diluted with ice water (100.0 mL). precipitated solid filtered and washed with diethyl ether and dried under vacuum to afford 3,7-bis(benzyloxy)-2-(4-(benzyloxy)-3-(trifluoromethyl)phenyl)-4H-chromen-4-one (5) as brown solid (2.8 g, 46%). ESI-MS: calculated m/z 608.2 [M]+, observed m/z 609.1 [M+H]+.

### 4. Synthesis of 3,7-bis(benzyloxy)-2-(4-(benzyloxy)-3-(trifluoromethyl)phenyl)-4H-chromene (6a) & 3,7-bis(benzyloxy)-2-(4-(benzyloxy)-3-(trifluoromethyl)phenyl)-2H-chromene (6b)

To a stirred solution of 3,7-bis(benzyloxy)-2-(4-(benzyloxy)-3-(trifluoromethyl)phenyl)-4H-chromen-4-one (5, 2.8 g, 4.6 mmol, 1.0 eq) in methyl tert-butyl ether (50 ml), lithium aluminium hydride (0.7 g, 18.4 mmol, 4.0 eq) was added at 40° C. under a nitrogen atmosphere. After stirring at this temperature for 10 min, the temperature of the reaction mixture was elevated to 80° C. for 2 h. After completion of the reaction, the reaction mixture was quenched with a cold water solution (2.0 ml) and diluted with ethyl acetate (100.0 mL). The organic layer was washed with water (20.0×2 mL) and brine (20 mL), dried over anhydrous sodium sulphate and evaporated under reduced pressure to afford crude. The crude was purified by column chromatography (100-200 silica mesh), and eluted with 20% ethyl acetate in hexane to afford mixture of 3,7-bis(benzyloxy)-2-(4-(benzyloxy)-3-(trifluoromethyl)phenyl)-4H-chromene (6a) and 3,7-bis(benzyloxy)-2-(4-(benzyloxy)-3-(trifluoromethyl)phenyl)-2H-chromene (6b) as brown sticky solid (1.0 g, 37% yield). ESI-MS: calculated m/z 594.2 [M]+, observed m/z 595.11 [M+H]+.

### 5. Synthesis of (2S,3S)-2-(4-hydroxy-3-(trifluoromethyl)phenyl)chromane-3,7-diol (Compound 77)

To a stirred solution of 3,7-bis(benzyloxy)-2-(4-(benzyloxy)-3-(trifluoromethyl)phenyl)-4H-chromene (6a) and 3,7-bis(benzyloxy)-2-(4-(benzyloxy)-3-(trifluoromethyl)phenyl)-2H-chromene (6b) (1.0 g, 1.68 mmol, 1.0 eq) in tetrahydrofuran (20 ml), Pd(OH)₂ (1.0 g) was added at room temperature. The suspension was hydrogenated in a par shaker pressure under 60 psi of hydrogen pressure for 3 h. After completion of the reaction, reaction was filtered over celite and concentrated to afford the crude product. The crude was purified by column chromatography (100-200 silica mesh) and eluted with 50% ethyl acetate in hexane, to afford 2-(4-hydroxy-3-(trifluoromethyl)phenyl)chromane-3,7-diol (7) (150 mg, 27%). ESI-MS: calculated m/z 326.29 [M]+, observed m/z 327.1 [M+H]+. The enantiomers were separated by chiral preparative HPLC [Chiralpak ADH (250×10) mm]. Peak-2 2-(4-hydroxy-3-(trifluoromethyl)phenyl)chromane-3,7-diol (Compound 77b): 38 mg; Rt 9.53 (ee>99%); HPLC purity 98.8%; 1H NMR (300 MHz, DMSO-d6): δ/ppm 10.5 (bs, 1H), 9.1 (bs, 1H), 7.59 (s, 1H), 7.50-7.47 (d, J=9.0 Hz, 1H), 7.01-6.98 (d, J=9.0 Hz, 1H), 6.85-6.83 (d, J=6.0 Hz, 1H), 6.31-6.29 (d, J=6.0 Hz, 1H), 6.21 (s, 1H), 4.98 (s, 1H), 4.90-4.88 (d, J=6.0 Hz, 1H), 3.95 (s, 1H), 3.17-2.99 (m, 2H). ESI-MS: calculated m/z 326.29 [M]+, observed m/z 326.9 [M+H]+.

### Example 4: Synthesis of Compound 78

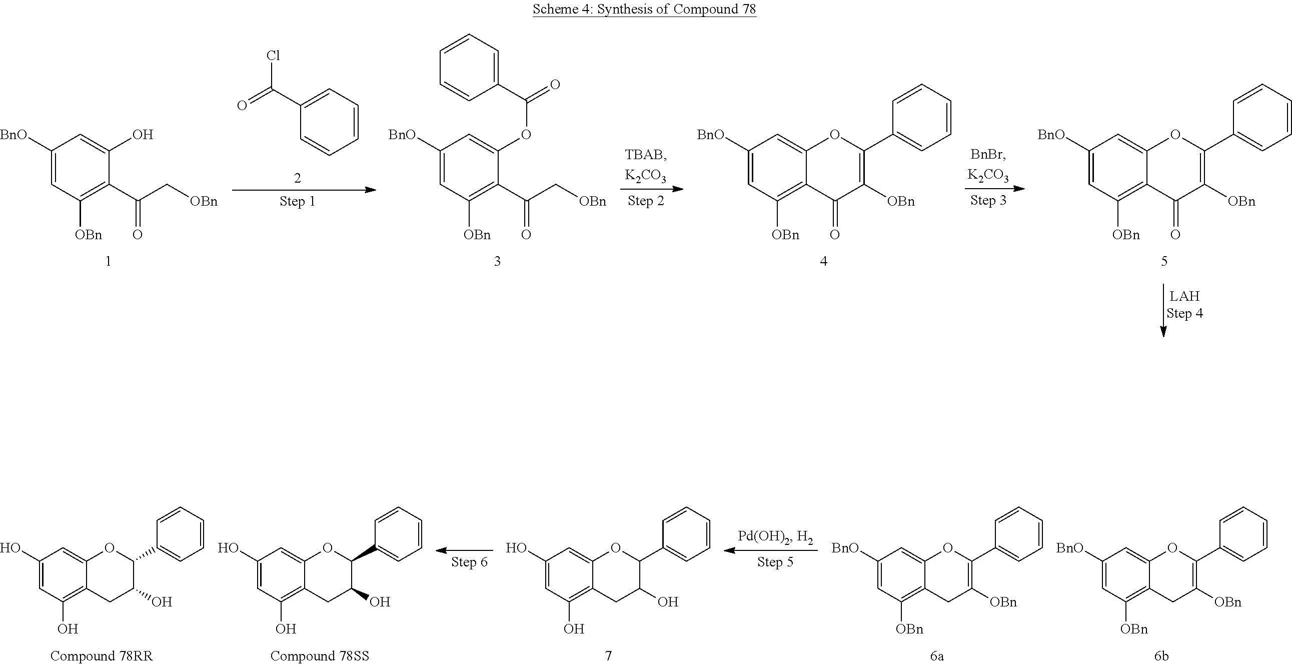

### 1. Synthesis of 3,5-bis(benzyloxy)-2-(2-(benzyloxy)acetyl)phenyl benzoate (3)

To a stirred solution of 2-(benzyloxy)-1-(2,4-bis(benzyloxy)-6-hydroxyphenyl)ethan-1-one (1, 16.0 g, 35.2 mmol, 1.0 eq), 4-dimethylaminopyridine (2.1 g, 17.62 mmol, 0.5 eq) and triethylamine (14.7 mL, 105.7 mmol, 3.0 eq) in dry dichloromethane (50.0 mL), benzoyl chloride (2, 5.92 g, 42.2 mmol, 1.2 eq) was added at 0° C. and stir at room temperature for 3 h. After completion of the reaction, the reaction mixture was diluted with water (100 mL) extracted with ethyl acetate (100 mL), dried over anhydrous sodium sulphate, and concentrated. The crude product was purified by column chromatography (100-200 silica mesh), using 30% ethyl acetate in hexane to obtain 3,5-bis(benzyloxy)-2-(2-(benzyloxy)acetyl)phenyl benzoate (3) as a dark brownish sticky compound (18.7 g, 95%). ESI-MS: calculated m/z 558.2 [M]+, observed m/z 559.0 [M+H]+.

### 2. Synthesis of 3,7-bis(benzyloxy)-5-hydroxy-2-phenyl-4H-chromen-4-one (4)

To a stirred solution of 3,5-bis(benzyloxy)-2-(2-(benzyloxy)acetyl)phenyl benzoate (3, 18.7 g, 33.5 mmol, 1.0 eq) in toluene (150 mL), tetrabutylammonium bromide (12.9 g, 40.20 mmol, 1.2 eq) and potassium carbonate (18.5 g, 134.0 mmol, 4.0 eq) were added and refluxed at 90° C. for 4 hrs. After completion of the reaction, the mixture was evaporated and diluted with dichloromethane (100 mL). The organic layer washed with water and brine, dried over anhydrous sodium sulphate. The crude product was purified by column chromatography (100-200 silica mesh) using 30% ethyl acetate in hexane to afford 3,7-bis(benzyloxy)-5-hydroxy-2-phenyl-4H-chromen-4-one (4), as a brown sticky compound (23.02 g, 88%). ESI-MS: calculated m/z 450.15 [M]+, observed m/z 451.2 [M+H]+.

### 3. Synthesis of 3,5,7-tris(benzyloxy)-2-phenyl-4H-chromen-4-one (5)

To a stirred solution of 3,7-bis(benzyloxy)-5-hydroxy-2-phenyl-4H-chromen-4-one (4, 23.0 g, 50.87 mmol, 1.0 eq) in N,N′-dimethylformamide (100 mL), benzyl bromide (13.0 g, 76.28 mmol, 1.5 eq) and potassium carbonate (28.1 g, 203.6 mmol, 4.0 eq) were added and stirred at room temperature for 16 hrs. After completion of the reaction, the reaction mixture was diluted with dichloromethane (100 mL) and washed with brine, and dried over anhydrous sodium sulphate. The organic layer was concentrated to obtain the crude. The crude product was purified by column chromatography (100-200 silica mesh) using 30% ethyl acetate in hexane to obtain 3,5,7-tris(benzyloxy)-2-phenyl-4H-chromen-4-one (5), as a brown solid (13.02 g, 56.56%). ESI-MS: calculated m/z 540.19 [M]+, observed m/z 541.2 [M+H]+.

### 4. Synthesis of 3,5,7-tris(benzyloxy)-2-phenyl-4H-chromene (6a) & 3,5,7-tris(benzyloxy)-2-phenyl-4H-chromene (6b)

To a stirred solution of 3,5,7-tris(benzyloxy)-2-phenyl-4H-chromen-4-one (5, 10.0 g, 19.01 mmol, 1.0 eq) in methyl tertiary-butyl ether (100 mL), lithium aluminium hydride (2.1 g, 57.02 mmol, 3.0 eq) was added at 50° C. After addition, the reaction mixture was stirred at 80° C. for 3 hrs. After completion of the reaction, the reaction mixture was quenched with ice water (50 mL), extracted with ethyl acetate (100 mL), washed with brine, and dried over anhydrous sodium sulphate. The organic layer was concentrated to obtain the crude. The crude product was purified by column chromatography (100-200 silica mesh) using 20% ethyl acetate in hexane to obtain a mixture of 3,5,7-tris(benzyloxy)-2-phenyl-4H-chromene (6a) and 3,5,7-tris(benzyloxy)-2-phenyl-4H-chromene (6b) as a white greasy liquid (8.0 g, 80%). ESI-MS: calculated m/z 526.2 [M]+, observed m/z 527.1 [M+H]+.

### 5. Synthesis of (2R,3R)-2-phenylchromane-3,5,7-triol (Compound 78)

To a stirred solution of 3,5,7-tris(benzyloxy)-2-phenyl-4H-chromene (6a) and 3,5,7-tris(benzyloxy)-2-phenyl-4H-chromene (6b) (8.0 g, 15.62 mmol) in mixture of tetrahydrofuran and ethanol (1:1, 52 ml), Pd(OH)₂ (5.0 g) was added at room temperature. The suspension was hydrogenated in a par shaker pressure under 60 psi for 3 h. The reaction was filtered over celite and concentrated to afford the crude product. The crude was purified by column chromatography (100-200 silica mesh) using 50% ethyl acetate in hexane, to afford 2-phenylchromane-3,5,7-triol (7) (900 mg, 11.25%). The enantiomers were separated by chiral preparative HPLC [Chiralpak ADH (250×10) mm]. Peak-1 2-phenylchromane-3,5,7-triol (Compound 78a), 50.86 mg; Chiral RT 9.567, ee>100%; HPLC purity: 99.96%; 1H NMR (300 MHz, DMSO-d6): δ/ppm 9.16 (s, 1H), 8.94 (s, 1H), 7.43-7.41 (m, 2H), 7.36-7.24 (m, 3H), 5.91-5.90 (d, J=2.1 Hz, 1H), 5.758-5.750 (d, J=2.4 Hz, 1H), 4.92 (s, 1H), 4.77-4.76 (d, J=4.5 Hz, 1H), 4.09-4.08 (bs, 1H), 2.75-2.69 (m, 2H); ESI-MS: calculated m/z 258.09 [M]+, observed m/z 259.3 [M+H]+. Peak-2 2-phenylchromane-3,5,7-triol (Compound 78b)), 58.45 mg; chiral RT 14.79, ee>99.9%; HPLC purity: 99.96%; 1H NMR (300 MHz, DMSO-d6): δ/ppm 9.16 (s, 1H), 8.94 (s, 1H), 7.43-7.41 (m, 2H), 7.36-7.27 (m, 3H), 5.91-5.90 (d, J=2.1 Hz, 1H), 5.758-5.750 (d, J=2.4 Hz, 1H), 4.92 (s, 1H), 4.77-4.76 (d, J=4.5 Hz, 1H), 4.09-4.08 (bs, 1H), 2.75-2.69 (m, 2H); ESI-MS: calculated m/z 258.09 [M]+, observed m/z 259.1 [M+H]+.

### Example 5: Characterization of Compounds

Additional compounds were prepared. Their characterization data is presented in Table 2.

TABLE 2
Characterization of prepared compounds.

| Compound | LCMS/1H NMR |
|---|---|
| Cis-2-(pyridin-4-yl)-3,4-dihydro-2H-1- | MS (ESI): calculated m/z 259.08 [M]⁺, |
| benzopyran-3,5,7-triol | observed m/z 260.0 [M + 1]⁺; HPLC purity: |
| (single enantiomer) | 98.03%. Chiral RT 38.81, ee = 100%. 1H |
| | NMR (300 MHz, DMSO-d6): δ/ppm 9.31 |
| | (s, 1H), 9.06 (s, 1H), 8.56-8.54 (d, J = 6 |
| | Hz, 2H), 7.38-7.36 (d, J = 6 Hz, 2H), 5.93- |
| | 5.92 (d, J = 3 Hz, 1H), 5.76 (d, J = 3 Hz, |
| | 1H), 5.21-5.20 (d, J = 3 Hz, 1H), 4.73-4.71 |
| | (d, J = 6 Hz, 1H), 3.90-3.86 (t, 1H), 2.67- |
| | 2.60 (m, 1H), 2.41-2.35 (m, 1H). |
| Cis-2-(2-hydroxypyridin-3-yl)chromane- | ESI-MS: calculate m/z 275.0 [M]⁺, |
| 3,5,7-triol | observed m/z: 275.9 [M + 1]⁺; HPLC |
| (single enantiomer) | purity: 89.9%. Chiral Rt 18.6 (ee = 99%). |
| | 1H NMR (300 MHz, DMSO-d6): δ/ppm |
| | 11.71 (bs, 1H), 9.17 (s, 1H), 8.96 (s, 1H), |
| | 7.36-7.26 (m, 2H), 6.19-6.15 (m, 1H), |
| | 5.89-5.88 (d, J = 3.1 Hz, 1H), 5.71-5.70 (d, |
| | J = 3.0 Hz, 1H), 5.02-5.00 (d, J = 5.9 Hz, |
| | 1H), 4.92-4.90 (d, J = 6.0 Hz, 1H), 4.19- |
| | 4.15 (m, 1H), 2.40-2.39 (m, 2H). |
| Cis-2-(5-fluoropyridin-2-yl)chromane- | MS (ESI): calculated m/z 277.08[M]⁺, |
| 3,5,7-triol | observed m/z 278.1[M + 1]⁺; Chiral RT |
| (single enantiomer) | 12.34, ee = 100%. ¹H NMR (300 MHz, |
| | DMSO-d6): δ/ppm 9.21 (s, 1H), 8.98 (s, |
| | 1H), 8.55 (s, 1H), 7.75 (m, 1H), 7.62-7.60 |
| | (m, 1H), 5.93 (s, 1H), 5.79 (s, 1H), 4.91 (br |
| | s, 1H), 4.76-4.74 (d, J = 6 Hz, 1 H), 4.29 (br |
| | s, 1H). |
| Cis-2-(5-fluoropyridin-2-yl)chromane- | MS (ESI): calculated m/z 277.08[M]⁺, |
| 3,5,7-triol | observed m/z 278.3[M + 1]⁺; Chiral RT |
| (single enantiomer) | 26.11, ee = 100%. ¹H NMR (300 MHz, |
| | DMSO-d6): δ/ppm 9.21 (s, 1H), 8.98 (s, |
| | 1H), 8.55 (s, 1H), 7.76 (m, 1H), 7.62-7. 60 |
| | (m, 1H), 5.93 (s, 1H), 5.79 (s, 1H), 4.91 (br |
| | s, 1H), 4.76-4.74 (d, J = 6 Hz, 1 H), 4.29 (br |
| | s, 1H). |
| Cis-4-(3-hydroxy-3,4-dihydro-2H- | ESI-MS (m/z): Calcd for C₁₄H₁₃NO₄: |
| pyrano[3,2-b]pyridin-2-yl)benzene-1,2-diol | 259.08, Observed 259.90 [M + H]⁺. HPLC |
| (single enantiomer) | purity: 99.85%. Chiral Rt 21.05, ee > 99%. |
| | ¹H NMR (300 MHz, DMSO-d6): δ/ppm |
| | 8.87 (s, 1H), 8.79 (s, 1H), 8.06 (d, J = 4.5 |
| | Hz, 1H), 7.18-7.13 (m, 2H), 6.93 (s, 1H), |
| | 6.69 (bs, 2H), 4.99-4.96 (m, 2H), 4.14 |
| | (bs, 1H), 2.83 (bs, 1H), 2.77 (bs, 1H). |
| Cis-4-(3-hydroxy-3,4-dihydro-2H- | ESI-MS (m/z): Calcd for C₁₄H₁₃NO₄: |
| pyrano[3,2-b]pyridin-2-yl)benzene-1,2-diol | 259.08, Observed 259.90 [M + H]⁺. HPLC |
| (single enantiomer) | purity: 96.56%. Chiral Rt 13.38, ee > 97%. |
| | ¹H NMR (300 MHz, DMSO-d6): δ/ppm |
| | 8.88 (s, 1H), 8.80 (s, 1H), 8.07 (d, J = 2.7 |
| | Hz, 1H), 7.17-7.14 (m, 2H), 6.94 (s, 1H), |
| | 6.70 (bs, 2H), 5.00-4.97 (m, 2H), 4.16 |
| | (bs, 1H), 2.83-2.77 (m, 1H), 2.61 (bs, |
| | 1H). |
| Cis-2-(3-fluoro-4-hydroxyphenyl)-3,4- | ESI-MS: Calculated m/z 261.08 [M]⁺, |
| dihydro-2H-pyrano[3,2-b]pyridin-3-ol | observed m/z 262.1 [M + H]⁺. Chiral RT |
| (single enantiomer) | 16.436 (ee > 99%); HPLC purity 94.43%; |
| | ¹H NMR (300 MHz, DMSO-d6): δ/ppm |
| | 9.82 (s, 1H), 8.09-8.07 (d, J = 6.0 Hz, 1H), |
| | 7.26-7.06 (m, 5H), 6.96-6.90 (m, 1H), |
| | 5.08-5.07 (d, 2H), 4.19 (bs, 1H), 2.84 (s, |
| | 1H), 2.80 (s, 1H). |
| Trans-2-(3-hydroxyphenyl)-3,4-dihydro-2H- | MS (ESI): calculated mass: 243.08 [M]⁺, |
| pyrano[3,2-b]pyridin-3-ol | observed m/z: 244.1 [M + 1]⁺; HPLC purity: |
| (single enantiomer) | 98.92%, Chiral RT 12.915, ee > 99%. ¹H |
| | NMR (300 MHz, DMSO-d6): δ/ppm 9.43 |
| | (s, 1H), 8.08-8.07 (d, J = 3 Hz, 1H), 7.26- |
| | 7.12(m, 3H), 6.75-6.67 (m, 3H), 5.34-5.32 |
| | (d, J = 6 Hz, 1H), 4.93-4.91 (d, J = 6 Hz, |
| | 1H), 4.16-4.13 (t, 1H), 2.85 (s, 2H). |
| Cis-2-(3-hydroxyphenyl)-3,4-dihydro-2H- | MS (ESI): calculated m/z 243.08 [M]⁺, |
| pyrano[3,2-b]pyridin-3-ol | observed m/z 244.3 [M + 1]⁺; HPLC purity: |
| (single enantiomer) | 93.17%.Chiral RT 13.937, ee > 94%. ¹H |
| | NMR (300 MHz, DMSO-d6): δ/ppm 9.35 |
| | (s, 1H), 8.09-8.08 (d, J = 3 Hz, 1H), 7.23- |
| | 7.12(m, 3H), 6.93-6.85 (m, 2H), 6.70- |
| | 6.66(m, 1H), 5.07-5.02 (m, 2H), 4.22 (s, |
| | 1H), 2.84 (s, 1H), 2.79(s, 1H). |
| Cis-4-(3-hydroxy-5,7-dimethoxy-1,2,3,4- | ESI-MS (m/z): Calcd for C₁₇H₁₉NO₅: |
| tetrahydroquinolin-2-yl)benzene-1,2-diol | 317.13, Observed 317.90 [M + H]⁺. HPLC |
| (single enantiomer) | purity: 83.87%. Chiral Rt 16.38, ee > 99%. |
| | ¹H NMR (300 MHz, DMSO-d6): δ/ppm |
| | 8.81 (s, 1H), 8.68 (s, 1H), 6.69-6.64 (m, |
| | 2H), 6.55-6.53 (m, 1H), 6.41-6.36 (m, |
| | 1H), 5.85 (bs, 1H), 5.75-5.74 (m, 1H), |
| | 4.59 (d, J = 4.8 Hz, 1H), 3.85 (d, J = 6.3 |
| | Hz, 2H), 3.67 (s, 3H), 3.63 (s, 3H), 2.91- |
| | 2.80 (m, 1H). |
| Cis-2-(3-fluoro-4-hydroxyphenyl)-1,2,3,4- | ESI-MS (m/z): Calcd for C₁₅H₁₄FNO₂: |
| tetrahydroquinolin-3-ol | 259.10, Observed 260.10 [M + H]⁺. HPLC |
| (single enantiomer) | purity: 99.64%. Chiral Rt 30.37, ee > 99%. |
| | ¹H NMR (300 MHz, DMSO-d6): δ/ppm |
| | 9.69 (s, 1H), 8.85 7.05-6.84 (m, 5H), 6.55 |
| | (d, J = 7.5 Hz, 1H), 6.46-6.44 (m, 1H), |
| | 5.94 (s, 1H), 4.80 (d, J = 4.8 Hz, 1H), 4.0 |
| | (d, J = 6.9 Hz, 1H), 3.76-3.73 (m, 1H), |
| | 2.80-2.68 (m, 2H). |
| Cis-2-(3-fluoro-4-hydroxyphenyl)-1-methyl- | MS (ESI): calculated m/z 273.12 [M]⁺, |
| 1,2,3,4-tetrahydroquinolin-3-ol | observed m/z 272.0 [M + 1]⁺; HPLC purity: |
| (single enantiomer) | 99.75%, Chiral RT 26.11, ee = 99.6%. ¹H |
| | NMR (300 MHz, DMSO-d6): δ/ppm 9.72 |
| | (s, 1H), 7.08-7.03 (t, 1H), 6.97-6.70 (m, |
| | 4H), 6.64-6.50 (m, 2H), 4.98-4.95 (t, 1H), |
| | 4.20 (s, 1H), 3.89 (br s, 1H), 2.79 (s, 3H). |
| Cis-2-cyclohexylchromane-3,5,7-triol | MS (ESI): calculated m/z 264.08 [M]⁺, |
| (single enantiomer) | observed m/z 265.1 [M + 1]⁺; HPLC purity: |
| | 98.37%, Chiral RT 9.99, ee > 100%. ¹H |
| | NMR (300 MHz, DMSO-d6): δ/ppm 9.03 |
| | (s, 1H), 8.81 (s, 1H), 5.83-5.82 (d, J = 3 |
| | Hz, 3H), 5.609-5.603 (m, J = 1.8 Hz, 3H), |
| | 4.55-4.54 (d, J = 3 Hz, 1H), 4.04 (bs, 1H), |
| | 3.29 (S, 2H), 2.14-2.09 (bs, 2 Hz), 1.80- |
| | 1.65 (m, 7H), 1.33-1.17(m, 9H), 0.92- |
| | 0.83 (m, 4H). |
| Cis-2-nonylchromane-3,5,7-triol | ESI-MS (m/z): Calcd for C₁₈H₂₈O₄: |
| (single enantiomer) | 308.20, Observed 309.00 [M + H]⁺. HPLC |
| | purity: 99.96%. Chiral Rt 13.89, ee > 99%. |
| | ¹H NMR (300 MHz, DMSO-d6): δ/ppm |
| | 9.08 (s, 1H), 8.85 (s, 1H), 5.83 (d, J = 2.1 |
| | Hz, 1H), 5.60 (d, J = 2.1 Hz, 1H), 4.73 (d, |
| | J = 5.4 Hz, 1H), 3.86 (bs, 1H), 3.73-3.71 |
| | (m, 1H), 1.56-1.52 (m, 2H), 1.40-1.25 |
| | (m, 16H), 0.97-0.80 (m, 3H). |
| Cis-2-(tetrahydro-2H-pyran-4-yl)chromane- | ESI-MS: calculated m/z 267.1 [M]⁺, |
| 3,5,7-triol | observed MS m/z 267.1 [M + 1]⁺; HPLC |
| (single enantiomer) | purity: 98.9%. Chiral Rt 14.801 (ee = |
| | 100%). 1H NMR (300 MHz, DMSO-d6): |
| | δ/ppm 9.05 (s, 1H), 8.83 (s, 1H), 5.84-5.83 |
| | (d, J = 3 Hz, 1H), 5.618-5.611 (d, J = 2.1 |
| | Hz, 1H), 4.651-4.637 (d, J = 4.2 Hz, 1H), |
| | 4.114-3.864 (m, 4H), 2.727 (s, 1H), 2.271 |
| | (s, 2H), 1.957-1.917 (d, J = 12 Hz, 2H), |
| | 1.647-1.603 (d, J = 13.2 Hz, 1H), 1.333- |
| | 1.106 (m, 3 H). |
| Cis-2-cyclohexylchromane-3,5,7-triol | MS (ESI): calculated m/z 264.08 [M]⁺, |
| (single enantiomer) | observed m/z 265.1 [M + 1]⁺; HPLC purity: |
| | 98.53%, Chiral RT 13.37, ee > 97.5%. ¹H |
| | NMR (300 MHz, DMSO-d6): δ/ppm 9.03 |
| | (s, 1H), 8.81 (s, 1H), 5.83-5.82 (d, J = 3 |
| | Hz, 3H), 5.609-5.603 (m, J = 1.8 Hz, 3H), |
| | 4.55-4.54 (d, J = 3 Hz, 1H), 4.04 (bs, 1H), |
| | 3.29 (S, 2H), 2.14-2.09 (bs, 2 Hz), 1.80- |
| | 1.65 (m, 7H), 1.33-1.17(m, 9H), 0.92- |
| | 0.83 (m, 4H). |
| Cis-2-(tetrahydro-2H-pyran-4-yl)chromane- | ESI-MS: calculated m/z 266.1 [M]⁺, |
| 3,5,7-triol | observed m/z: 267.1 [M + 1]⁺; HPLC |
| (single enantiomer) | purity: 98.7%. Chiral Rt 22.889 (ee |
| | 99.9%). ¹H NMR (300 MHz, DMSO-d6): |
| | δ/ppm 9.058 (s, 1H), 8.832 (s, 1H), 5.847- |
| | 5.840 (d, J = 2.1 Hz, 1H), 5.619-5.611 (d, |
| | J = 2.4 Hz, 1H), 4.651-4.637 (d, J = 4.2 Hz, |
| | 1H), 4.028-3.860 (m, 4 H), 2.728 (s, 1H), |
| | 2.272 (s, 2H), 1.959-1.915 (d, J = 12 Hz, |
| | 2H), 1.645-1.608 (d, J = 11.1 Hz, 1H), |
| | 1.295-1.205 (m, 3 H). |
| Cis-2-(4-fluoro-3-methoxyphenyl)chroman- | ESI-MS: calculated m/z 274.29 [M]⁺, |
| 3-ol | observed m/z 272.9 [M − H]⁻. HPLC purity: |
| (single enantiomer) | 99.9%; Chiral RT: 12.91 (ee = 99.71%). |
| | 1H NMR (300 MHz, DMSO-d6): δ/ppm |
| | 7.28-7.04 (m, 5H), 6.85-6.82 (m, 2H), 5.06 |
| | (s, 1H), 4.93-4.92 (d, J = 4.6 Hz, 1H), 4.15 |
| | (bs, 1H), 3.83 (s, 3H), 3.21-3.16 (d, J = 15 |
| | Hz, 1H), 2.74-2.69 (d, J = 15 Hz, 1H). |
| Cis-2-(3-fluoro-4-hydroxyphenyl)chromane- | ESI-MS: calculated m/z 292.1 [M]⁺, |
| 3,5,7-triol | observed m/z 293.21[M + 1]⁺. Chiral Rt |
| (single enantiomer) | 20.65 (ee = 99.9%); HPLC purity 99.16%; |
| | 1H NMR (300 MHz, DMSO-d6): δ/ppm |
| | 9.75 (s, 1H), 9.16 (s, 1H), 8.94 (s, 1H), |
| | 7.19-7.15 (d, J = 12.6 Hz, 1H), 7.04-7.01 |
| | (d, J = 8.4 Hz, 1H), 6.92-6.86 (m, 1H), |
| | 5.90-5.89 (d, J = 2.1 Hz, 1H), 5.73-5.72 (d, |
| | J = 1.8 Hz, 1H), 4.82 (s, 1H), 4.79-4.78 (d, |
| | J = 3 Hz, 2H), 4.03 (bs, 1H), 2.72 (m, 1H), |
| | 2.27 (m, 1H). |
| Cis-2-(4-hydroxy-3- | ESI-MS: calculated m/z 326.29 [M]⁺, |
| (trifluoromethyl)phenyl)chromane-3,7-diol | observed m/z 326.9 [M + H]⁺. Chiral Rt 9.53 |
| (single enantiomer) | (ee = 99%); HPLC purity 98.8%; 1H NMR |
| | (300 MHz, DMSO-d6): δ/ppm 10.5 (bs, |
| | 1H), 9.1 (bs, 1H), 7.59 (s, 1H), 7.50-7.47 |
| | (d, J = 9.0 Hz, 1H), 7.01-6.98 (d, J = 9.0 |
| | Hz, 1H), 6.85-6.83 (d, J = 6.0 Hz, 1H), |
| | 6.31-6.29 (d, J = 6.0 Hz, 1H), 6.21 (s, 1H), |
| | 4.98(s, 1H), 4.90-4.88 (d, J = 6.0 Hz, 1H), |
| | 3.95 (s, 1H), 3.17-2.99 (m, 2H). |
| Cis-2-phenylchromane-3,5,7-triol | ESI-MS: calculated m/z 258.09 [M]⁺, |
| (single enantiomer) | observed m/z 259.3 [M + H]⁺. Chiral RT |
| | 9.567 (ee = 100%); HPLC purity: 99.96%; |
| | 1H NMR (300 MHz, DMSO-d6): δ/ppm |
| | 9.16 (s, 1H), 8.94 (s, 1H), 7.43-7.41 (m, |
| | 2H), 7.36-7.24 (m, 3H), 5.91-5.90 (d, J = |
| | 2.1 Hz, 1H), 5.758-5.750 (d, J = 2.4 Hz, |
| | 1H), 4.92 (s, 1H), 4.77-4.76 (d, J = 4.5 Hz, |
| | 1H), 4.09-4.08 (bs, 1H), 2.75-2.69 (m, 2H). |
| Cis-2-(4-hydroxy-3- | ESI-MS: calculated m/z 342.07 [M]⁺, |
| (trifluoromethyl)phenyl)chromane- | observed m/z 342.9 [M + H]⁺; HPLC purity: |
| 3,5,7-triol | 96%. Chiral Rt 11.98 (ee = 100%). 1H |
| (single enantiomer) | NMR (300 MHz, DMSO-d6): δ/ppm 10.49 |
| | (s, 1H), 9.19 (s, 1H), 8.96 (s, 1H), 7.59 (s, |
| | 1H), 7.50-7.47 (d, 1H), 7.00-6.98 (d, 1H), |
| | 5.90 (s, 1H), 5.73 (s, 1H), 4.89 (s,1H), |
| | 4.84-4.82 (d, 1H), 4.03 (s, 1H), 2.72-2.69 |
| | (d, 2H). |

### Example 6: In Vitro PGC-1α Expression Studies

Culture conditions: DMEM containing 25 mM glucose with 10% FBS supplementation; grown at 37° C. and 5% CO₂.

Drugs: (+)Epicatechin, AICAR (direct activator of AMPK).

Assay Conditions: C2C12 cells were cultured in DMEM supplemented in 10% FBS up to 80% confluence. The cells were further trypsinized and seeded into a 96 well plate at a density of 5000 cells/well (well volume—100 μL) (Day 0). Following attachment, the cells were allowed to reach 80-90% confluence in the plate (typically 48 hours post seeding) and further differentiated using DMEM+2% Horse Serum (200 μL/well). The media was changed every day or at least every alternate day for 5 days to allow myoblasts to differentiate into myotubes. On day 7 (post seeding), the cells were treated with standards and test compounds for PGC-1 α assay.

PGC-1α assay: For the detection of PGC-1α, the cells were incubated with 0.5 μg/ml primary antibody (Merck, WH0010891M3) in PBS-T containing 5% BSA at 4° C. overnight. The cells were then washed three times with PBS-T for 5 minutes and incubated with 1:1000 dilution secondary antibody (Anti-rabbit IgG, HRP-linked Antibody, Cell Signaling) in PBS-T with 1% BSA for 1 hour at RT. Cells were washed three times with PBS-T for 5 minutes The cells were incubated with 100 μl TMB substrate solution for 30 minutes and the reaction was stopped with 100 μl of 2N H₂SO₄. Then the plate was read at 450 nM using ELISA plate reader and absorbance recorded. % activity was calculated using DMSO control as 100%. (For plate optical density calculations, a background correction was taken at 540 nm). The final PGC-1α activation % is average of activation of 6 replicates.

TABLE 3
Effect of Compound 75b on % PGC-1α expression
in differentiated C2C12 cells (OD@650 nm).

| | | |
|---|---|---|
| Treatment | Concentration | % Activity |

| | | | |
|---|---|---|---|
| Epicatechin | 0.001 | nM | 119 |
| Compound 75b | 1 | nM | 120 |
| | 0.1 | nM | 116 |
| | 0.01 | nM | 122 |
| | 0.001 | nM | 125 |
| | 0.0001 | nM | 118 |

TABLE 4
Effect of Compound 75b on PGC-1α expression in differentiated
C2C12 cells (OD @ 650 nm) (OD at 5 minutes).

| | | |
|---|---|---|
| Treatment | Concentration | % Activity |

| | | | |
|---|---|---|---|
| AICAR | 1 | mM | 128 |
| Epicatechin | 0.001 | nM | 120 |
| Compound 75b | 0.01 | nM | 130 |

TABLE 5
Effect of Compound 76b on PGC-1α expression in differentiated
C2C12 (OD @ 650 nm) (5000 cells/well)

| | | |
|---|---|---|
| Treatment | Concentration | % Activity |

| | | | |
|---|---|---|---|
| DMSO | | | 100 |
| AICAR | 1 | mM | 122 |
| Epicatechin | 0.001 | nM | 133 |
| Compound 76b | 0.01 | nM | 128 |

TABLE 6
Effect of Compound 77b on PGC-1α expression in C2C12
cells (OD @ 650 nm) (5000 cells/well)

| | | |
|---|---|---|
| Treatment | Concentration | % Activity |

| | | | |
|---|---|---|---|
| AICAR | 1 | mM | 136 |
| Epicatechin | 0.001 | nM | 127 |
| Compound 77b | 0.01 | nM | 138 |

TABLE 7
Effect of Epicatechin and Compound 77b on PGC-1α expression
in C2C12 cells (OD @ 650 nm) (5000 cells/well)

| | | |
|---|---|---|
| Treatment | Concentration | % Activity |

| | | | |
|---|---|---|---|
| DMSO | | | 100 |
| AICAR | 1 | mM | 122 |
| Epicatechin | 0.001 | nM | 133 |
| Compound 77b | 0.01 | nM | 135 |

TABLE 8
Effect of Compound 78a on PGC-1α expression in C2C12
cells (OD @ 650 nm) (5000 cells/well)

| | | |
|---|---|---|
| Treatment | Concentration | % Activity |

| | | | |
|---|---|---|---|
| DMSO | | | 100 |
| AICAR | 1 | mM | 140 |
| Epicatechin | 0.001 | nM | 139 |
| Compound 78a | 0.01 | nM | 128 |

### Example 7: In Vivo PGC-1α Expression Studies

C57BL/6 mice were procured from Vivo Bio Tech Ltd. Male mice were used for the study within an age range of 7 to 8 weeks. Body weight for these mice ranged from 20 g to 25 g. Animals were housed in a group of 6 in individually ventilated cages with temperature of 22±3° C., a relative humidity of 50 to 60%. The bottom of the cages was layered with sterilized corn cob. Animals were provided with gamma irradiated feed and autoclaved drinking water ad-libitum. The IVCs were maintained at an environment controlled (Centrally Air-Conditioned) room at a temperature of 22±3° C., a relative humidity of 50 to 60%, light intensity of 250-300 lux and a 12 hour light-dark cycle and noise intensity of <85 db.

Method of Preparation: LPS was dosed at 3 mg/kg in a dose volume of 10 ml/kg. For this, 0.3 mg/ml of LPS solution (Micronized suspension) was prepared. Butter paper was placed on analytical balance, tarred and LPS was placed on butter paper and its weight was noted down. LPS was transferred to 15 ml falcon tube. Then required amount of normal saline was added to it to make it 0.3 mg/ml. The solution was vortexed properly for 5 minutes and placed on ice. LPS solution was vortexed every time before aspiration and injection.

LPS was dosed at 3 mg/kg. Each test compound was dosed at 10 mg/kg, 2 hour and 15 min post LPS administration.

0.5% CMC was used as a vehicle with a dose volume of 10 ml/kg. An IP route was used for LPS, and a PO route was used for each test compound. Animals were sacrificed 24 hours post LPS dosing, and plasma were collected. PGC-1α level was measured in liver samples, and other tissues were stored for future analysis. PGC-1α level was checked using ELISA kit—CUSABIO Mouse PPARGCIA ELISA kit.

Protocol: One day before start of the experiment C57BL/6 Mice (Male, 6-7-week-old) were randomized on the basis of their body weight and were divided into groups. All the cages were assigned the respective group name. Animals inside each cage were marked on the base of the tails with permanent marker to distinguish each animal. Mice were fasted overnight. Next day morning animals were dosed with test item. Group 1 (Control) and Group 2 (LPS) Animals were dosed with vehicle (0.5% CMC). Test items were prepared in 0.5% CMC by trituration in mortar and pestle and addition of CMC drop by drop. 2 hour post test item/vehicle dosing animals were administered with LPS through IP route at 3 mg/kg of body weight. Group 1 (Control) animals were administered with saline. 0.25 hour post LPS administration, animals were again dosed with test item/vehicle. Post dosing, animals were returned to their respective cages and kept until termination of the study. No feed was offered to any group. Animals were provided with water. 24 hour post LPS administration, blood was collected from each animal under influence of mild isoflurane anesthesia through retro-orbital technique. Animals were sacrificed by CO₂ asphyxiation technique. Post-termination, animals were dissected and vital organs, liver, brain, skeleton muscle were collected in Eppendorf tubes and flash frozen in liquid nitrogen. Serum was collected from whole blood by centrifuging it at 4° C. at 10,000 RPM and for 10 minutes. (Blood was left for 2 hour for coagulation before centrifugation) After completion of the experiment, all collected samples were stored at −80° C. until analysis.

Assay Methods/Protocol: The mortar was chilled with liquid nitrogen and then the small tissue pieces were grinded in the presence of liquid nitrogen to a fine powder. Immediately after grinding, transferred small amount of tissue powder to a 1.5 ml micro centrifuge tube containing 1.0 ml lysis buffer (RIPA buffer+Protease inhibitor). The samples were then incubated on ice for 2 hours. The samples were vortexed every 15 minutes. The samples were then centrifuged at 13000×g for 15 minutes at 2-8° C. After centrifugation the supernatant was removed carefully and pellet was discarded. The protein assay was performed using a Bio-Rad Protein Assay Dye Reagent Concentrate (#5000006).

BSA standard curve and protein estimation: The Bio-Rad dye reagent was prepared by diluting 1 part dye reagent with 4 parts of distilled water. The dilutions for BSA were prepared from 10 mg/ml stock. The range of the dilutions was 1.0 mg/ml to 0.0125 mg/ml. For protein estimation 10 μl of each standard or sample was added to 96 well plate. To this 200 μl of diluted dye reagent was added. The samples and reagent were mixed thoroughly using micro plate mixer. The samples were incubated at room temperature for 5 minutes. The plate was read at 595 nm using Spectramax Me5.

For PGC-1α assay ELISA, samples were diluted in such a way that the saturation in signal can be avoided. This was achieved by running a small experiment every time with 8 wells. The 3-4 dilutions concentrations of a sample with known protein concentration were prepared and PGC-1α assay was ran along with 3-4 standard dilutions with lowest and highest value (pg/ml). This was to decide on which dilution should be used for final assay. After the confirmation of the working dilution, all the samples were diluted accordingly, and PGC-1α assay was performed according to the kit protocol. The final values were multiplied by the dilution factor. The data was analyzed using the PGC-1α standard curve.

TABLE 9
in vivo PGC-1α expression studies.

| Groups (n = 6) | PGC-1α level (pg/ml) | PGC-1α level (%) |
|---|---|---|
| Control | 35303 | 100 |
| LPS (3 mg/Kg) | 18139 | 51 |
| LPS (3 mg/Kg) + | 31729 | 90 |
| Epicatechin (BID) | | |
| LPS (3 mg/Kg) + | 32354 | 92 |
| Compound 75b (BID) | | |
| Control | 25677 | 100 |
| LPS (3 mg/Kg) | 16956 | 66 |
| LPS (3 mg/Kg) + | 31225 | 122 |
| Epicatechin (BID) | | |
| LPS (3 mg/Kg) + | 37954 | 148 |
| Compound 77b (BID) | | |
| LPS (3 mg/Kg) + | 20790 | 81 |
| Compound 76b (BID) | | |
| No Saline Control | 24206 | 94 |
| Groups (n = 6) | PGC-1α levels (pg/ml) | PGC-1α level (%) |
| Control | 38327 | 100 |
| LPS (3 mg/Kg) | 16860 | 44 |
| LPS (3 mg/Kg) + | 27272 | 71 |
| Epicatechin (BID) | | |
| LPS (3 mg/Kg) + | 36967 | 96 |
| Compound 78a (BID) | | |

### Example 8: Pharmacokinetics of Compounds

C57BL/6 Mice, Age 7-9 weeks, body weight range 20-25 g were used. The mice were fasted overnight. Mice were dosed once a day for a single day at 10 mg/kg, 10 mg/kg. 6 time-points were used for whole blood. 10% NMP was used as a vehicle. 9 animals were used per treatment, with three groups of three animals each. Group 1 had time points at 15 and 60 minutes, Group 2 had time points at 30 and 120 minutes, and Group 3 had time points at 90 and 240 minutes. An Oral (PO) route was used. Plasma and brain samples were collected (brain sample collected at terminal time point). Plasma was collected through the centrifugation of whole blood at 10,000 rpm for 10 minutes at 4° C. Samples were stored at −80° C. until processing.

### LCMS-Plasma Method:

1.00 mg/mL in 100% MeOH PP vials (prepared at RT yellow light) at 2-8° C. were used as stock solution. 12.22-50000 ng/ml using 80% MeOH PP vials (prepared at RT yellow light) at 2-8° C. were used as working solution. 0.98-4000 ng/ml (prepared at RT yellow light) was used for the curve range. For Compounds 75b and 78a, 1.00 mg/mL of Compound 79 in 100% methanol Ria vials (Prepared at RT yellow light) was used as IS stock solution, and 60 ng/ml of Compound 79 in 80% methanol (Prepared at RT yellow light) was used as IS working solution. For Compound 76b, 1.00 mg/mL of Compound 44 in 100% methanol Ria vials (Prepared at RT yellow light) was used as IS stock solution, and 60 ng/ml of Compound 44 in 80% methanol (Prepared at RT yellow light) was used as IS working solution. A linear, 1/x2 fit type was used. The injection volume was 15 μL. Mobile Phase A was 0.1% Formic Acid in water at room temperature. Mobile Phase B was 100% methanol at room temperature. R0 wash solution was 80% methanol at room temperature. R³ wash solution was IPA/MeOH/ACN/water (1:1:1:1) at room temperature. The column was Agilent Eclipse 50*2.1 mm 1.8 μm and the instrument was Shimadzu LC-40 UPLC and AB Sciez 6500+ with LCMS002.

For Compound 75b, using a pipette with Methanol-rinsed pipette tips, 25 μL of each sample was aliquoted (calibration curve sample, QC samples and blank matrix) to labeled 12×75-mm glass tubes. Using Methanol-rinsed pipette tips, 5.0 μL of IS of conc. 60 ng (Compound 79) was aliquoted to each standards and samples except Blank. All tubes were mixed on a multi-tube vortex for approximately 30 seconds. 50 μL of 10 mM Sodium bicarbonate were added. 1 ml of Ethyl Acetate was added. All tubes were mixed on a multi-tube vortex for approximately 5 minutes then centrifuged for 10 min at 5,000 rpm, 4° C. The sample was kept in methanol containing dry ice for a few seconds to freeze the Aqueous layer (flash freeze method). The upper organic layer was poured from the frozen tubes into the labeled 12×75-mm glass tubes then evaporated to dryness under nitrogen using Liquid Evaporator (30° C., ˜15 psi, ˜10 mins). 200 μl of reconstitution solution (0.1% Acetic acid:MeOH (60:40 v/v) were added into each tube. Samples were vortexed for 30 seconds then transferred to amber glass injection vials for injection.

For Compounds 76b and 78a, using a pipette, 25 μL of each sample were aliquoted (calibration curve sample, QC samples and blank matrix) to labeled 1.5 ml eppendorfs. Using a pipette, 5.0 μL of IS of conc. 100 ng (Cocktail IS) was aliquoted to each standards and samples except Blank. All tubes were mixed on a multi-tube vortex for approximately 30 seconds. 1 ml of tert-butyl methyl ether (TBME) was added. All tubes were mixed on a multi-tube vortex for approximately 5 minutes then centrifuged for 10 min at 10,000 rpm, 4° C. The sample was kept in methanol containing dry ice for few seconds to freeze the Aqueous layer (flash freeze method). The upper organic layer was poured from the frozen tubes into the labeled 12×75-mm rial vials then vaporated to dryness under nitrogen using Liquid Evaporator (30° C., −15 psi, 10 mins). 200 μl of reconstitution solution (10 mM Ammonium Formate: MeOH) (40:60 v/v) was added into each tube. Samples were vortexed for 30 seconds then transferred to amber glass injection vials for injection.

After test item administration, blood samples were collected from individual animals in previously labeled Heparinised blood collection tubes. Blood samples were collected from retro orbital plexus. The time points for collection of blood samples from all the animals are; 0.25, 0.5, 1, 2 and 4 hr. The collected samples were subjected to centrifugation @ 10,000 rpm for 10 minutes at 40ºC for separation of plasma. Thereafter, plasma samples were collected in previously labeled plasma tubes. These were stored at −80° C. until analysis.

### LCMS-Brain Method

1.00 mg/mL in 100% MeOH PP vials (prepared at RT yellow light) at 2-8° C. were used as stock solution. 12.22-50000 ng/mL using 80% MeOH PP vials (prepared at RT yellow light) at 2-8° C. were used as working solution. 0.48-2000 ng/ml (prepared at RT yellow light) was used for the curve range. For Compounds 75b and 78a, 1.00 mg/mL of Compound 79 in 100% methanol Ria vials (Prepared at RT yellow light) was used as IS stock solution, and 60 ng/ml of Compound 79 in 80% methanol (Prepared at RT yellow light) was used as IS working solution. For Compound 76b, 1.00 mg/mL of Compound 44 in 100% methanol Ria vials (Prepared at RT yellow light) was used as IS stock solution, and 60 ng/ml of Compound 44 in 80% methanol (Prepared at RT yellow light) was used as IS working solution. A linear, 1/x2 fit type was used. The injection volume was 15 μL. Mobile Phase A was 0.1% Formic Acid in water at room temperature. Mobile Phase B was 100% methanol at room temperature. R0 wash solution was 80% methanol at room temperature. R³ wash solution was IPA/MeOH/ACN/water (1:1:1:1) at room temperature. The column was Agilent Eclipse 50*2.1 mm 1.8 μm and the instrument was Shimadzu LC-40 UPLC and AB Sciez 6500+ with LCMS002.

For Compound 75b, 100 mg of brain tissue were weighed, 400 μL of Ice cold PBS was added, and the sample was homogenated using a polytron homogenizer. Before starting the experiment the blade was cleaned with NS, Methanol, Methanol:Water (50:50) followed by NS. Using a pipette with Methanol-rinsed pipette tips, 50 μL of each sample (calibration curve sample, QC samples and blank matrix) was aliquot to labeled 12×75-mm glass tubes. Using Methanol-rinsed pipette tips, 5.0 μL of IS of conc. 60 ng (Compound 79) was added to each standard and samples except Blank. All tubes were mixed on a multi-tube vortex for approximately 30 seconds. 1 ml of Ethyl Acetate was added. All tubes were mixed on a multi-tube vortex for approximately 5 minutes then centrifuged for 10 min at 5,000 rpm, 4° C. The sample was kept in methanol containing dry ice for few seconds to freeze the Aqueous layer (flash freeze method). The upper organic layer was poured from the frozen tubes into the labeled 12×75-mm glass tubes then evaporated to dryness under nitrogen using Liquid Evaporator (30° C., −15 psi, ˜ 10 mins). 200 μl of reconstitution solution (10 mM Ammonium Formate:MeOH (40:60 v/v) was added into each tube, which was then vortexed for 30 seconds and transferred to amber glass injection vials for injection. At the terminal time points, animals were euthanized after blood sample collection. Their skulls were opened and whole brains were separated and collected in previously labeled tubes. The tubes were then immediately transferred into liquid nitrogen container for snap freezing. Later the samples were stored at −80° C.

For Compounds 76b and 78a, 100 mg of brain tissue were weighed, 400 μL of Ice cold PBS was added, and the samples were homogenated using a polytron homogenizer. Before starting the experiment the blade was cleaned with NS, Methanol, Methanol:Water (50:50) followed by NS. Using a pipette with Methanol-rinsed pipette tips, 50 μL of each sample (calibration curve sample, QC samples and blank matrix) was aliquoted to labeled 12×75-mm RIA vials. Using a pipette, 5.0 μL of IS of conc. 100 ng (Cocktail IS) was aliquoted to each standard and samples except Blank. All tubes were mixed on a multi-tube vortex for approximately 30 seconds. 1 ml of tert-butyl methyl ether (TBME) was added. All tubes were mixed on a multi-tube vortex for approximately 5 minutes then centrifuged for 10 min at 10,000 rpm, 4° C. The sample was kept in methanol containing dry ice for few seconds to freeze the Aqueous layer (flash freeze method). The upper organic layer was poured from the frozen tubes into the labeled 12×75-mm rial vials then evaporated to dryness under nitrogen using Liquid Evaporator (30° C., ˜15 psi, ˜10 mins). 200 μl of reconstitution solution (10 mM Ammonium Formate:MeOH (40:60 v/v) were added into each tube. The samples were Vortexed for 30 seconds then transferred to amber glass injection vials for injection. After test item administration, blood samples were collected from individual animals in previously labeled Heparinised blood collection tubes. Blood samples were collected from retro orbital plexus. The time points for collection of blood samples from all the animals are; 0.25, 0.5, 1, 2 and 4 hr. The collected samples were subjected to centrifugation @ 10,000 rpm for 10 minutes at 40ºC for separation of plasma. Thereafter, plasma samples were collected in previously labeled plasma tubes. These were stored at −80° C. until analysis.

TABLE 10
Quantitative Determination of Compounds in Mice Plasma.

| | Tmax | Cmax | AUC0-4 h |
|---|---|---|---|
| Compound | (hr) | (ng/ml) | (hr*ng/mL) |
| Compound 75b | 1.5 | 138.16 | 132.9172 |
| Compound 76b | 0.25 | 89.33 | 45.72235 |
| Compound 78a | 0.25 | 49.79 | 59.52464 |

TABLE 11
Quantitative Determination of Compounds in Mice Brains.

| | Tmax | Cmax | AUC0-4 |
|---|---|---|---|
| Compound | (hr) | (g/tissue) | (hr*g/tissue) |
| Compound 75b | 1 | 111.83 | 238.5399 |
| Compound 76b | 1 | 5.17 | 5.99257 |
| Compound 78a | 1 | 58.27 | 29.135 |

### Example 9: Hydrolysis Conditions

To assess whether the fraction of synthetic and reverse hydrolytic activities of mitochondrial ATP synthase can be measured in the same preparation, a protocol was developed to monitor changes in oxygen consumption and proton release in response to changes in mitochondrial ATP synthase activity. The proton released and the concomitant changes in the pH can be monitored using the ECAR (extracellular acidification) channel in the Agilent Seahorse XF analyzer using isolated mitochondria, to ensure that the changes in pH cannot be attributed to glycolysis or other contributors to ECAR.

Mice were anesthetized with isoflurane followed by a cervical dislocation, and the heart was immediately removed and placed in ice cold relaxation buffer (5 mM sodium pyrophosphate, 100 mM KCl, 5 mM EGTA, 5 mM HEPES; pH 7.4). The heart was squeezed with tweezers to remove blood, minced with scissors, and then placed in a glass-glass Dounce homogenizer with 3 mL of HES homogenization buffer (250 mM sucrose, 5 mM HEPES, 1 mM EDTA; pH to 7.2, adjusted with KOH). The heart tissue was homogenized first with the loose pestle and followed by the tight pestle. The homogenized tissue was placed in a pre-chilled 15 ml conical tube and centrifuged at 900×g (4° C.) for 10 minutes (min). The supernatant was removed, placed in a new tube and centrifuged again at 900×g for 10 min. The supernatant was then transferred to 2 mL microcentrifuge tubes and centrifuged at 10,000×g (4° C.) for 10 min. The mitochondrial pellets were re-suspended in ice cold HES buffer and mitochondrial protein was measured with a BCA assay (Pierce). The concentrated mitochondrial pellet was stored on ice.

Heart mitochondria (0.75-1.5 μg) were loaded into a Seahorse XF96 microplate in 20 μL of MAS (70 mM Sucrose, 220 mM Mannitol, 5 mM KH2PO4, 5 mM MgCl2, 1 mM EGTA, 2 mM HEPES; pH 7.2) plus 1% free fatty acid BSA containing substrates. The loaded plate was centrifuged at 2,000×g for 5 min at 4° C. (no brake) and an additional 130 μL of MAS was added to each well. When assessing compounds effect on respirometry, the compounds were added at this point at the indicated concentration in MAS buffer. To avoid disrupting mitochondrial adherence to the bottom of the plate, MAS was added using a multichannel pipette pointed at a 45° angle to the top of the well-chamber, as instructed by the manufacturer. Substrate concentrations in the well when assay was starting in state 4 were as follow: (i) 5 mM pyruvate+5 mM malate or ii) 5 mM succinate+2 μM rotenone. Substrate concentrations in the well when assay was starting in state 3 were as follow: (i) 5 mM pyruvate+5 mM malate+4 mM ADP or ii) 5 mM succinate+2 μM rotenone+4 mM ADP. Injections were performed as indicated in the figure descriptions at the following final concentration in the well: oligomycin (3.5 μM), FCCP (4 μM), Antimycin A (2 μM). Compounds were added at the indicated concentration.

ATP hydrolysis capacity or state 4 acidification was measured using Seahorse XF96 as described in Divakaruni et al. (2018) Anal Biochem 552:60-65 and Acin-Perez et al. (2021) Life 11(9):949, in MAS (70 mM Sucrose, 220 mM Mannitol, 5 mM KH₂PO₄, 5 mM MgCl₂, 1 mM EGTA, 2 mM HEPES; pH 7.2). Plates were loaded with 0.75-1.5 μg of mouse heart mitochondria, or 25 μg of cell lysate. Cell lysates were prepared by subjecting the samples to 4 cycles of free thaw (liquid nitrogen—37° C. water bath) before measuring protein concentration. When assessing compounds effect on respirometry, the compounds were added in MAS after sample centrifugation. Initial respiration of the samples was sustained by the addition of 5 mM succinate+2 μM rotenone in the MAS after centrifugation. Injections were performed at the following final concentration in the well: Antimycin A (2 μM), oligomycin (5 μM), FCCP (1 μM), ATP (20 mM). To assess maximal ATP concentration, ATP was injected consecutively.

Oxygen consumption was measured in isolated fresh intact mitochondria from mouse heart, as oxygen consumption is linked to maximal ATP synthesis and proton release when mitochondria are respiring either in the presence of substrates and ADP (ATP synthesizing mitochondria or state 3) or in the presence of substrates but not ADP (state 4). Oxygen consumption was fueled by Pyruvate plus Malate (Pyr+Mal) or Succinate plus Rotenone (Succ+Rot). Proton release in isolated mitochondria is mostly a result of ATP hydrolysis.

An additional assay was developed to determine the maximal ATP hydrolysis capacity in frozen mitochondria. Maximal ATP hydrolytic capacity was assessed in frozen heart mitochondria where mitochondrial respiration is inhibited by Antimycin A and maximal ATP hydrolysis is driven by co-injection of FCCP and ATP, followed by inhibition in the presence of oligomycin. Addition of oligomycin at different concentrations in the assay media revealed a dose dependent inhibition of ATP hydrolysis, demonstrating the specificity of the assay.

OCR and ECAR were measured in a Seahorse XF96 analyzer under basal conditions as well as after injection of 2 μM oligomycin, two sequential additions of 1.5 μM FCCP, followed by 1 μM rotenone with 2 μM antimycin A. Respiratory parameters were calculated according to standard protocols, and all rates were corrected for non-mitochondrial respiration/background signal by subtracting the oxygen consumption rate insensitive to rotenone plus antimycin A.

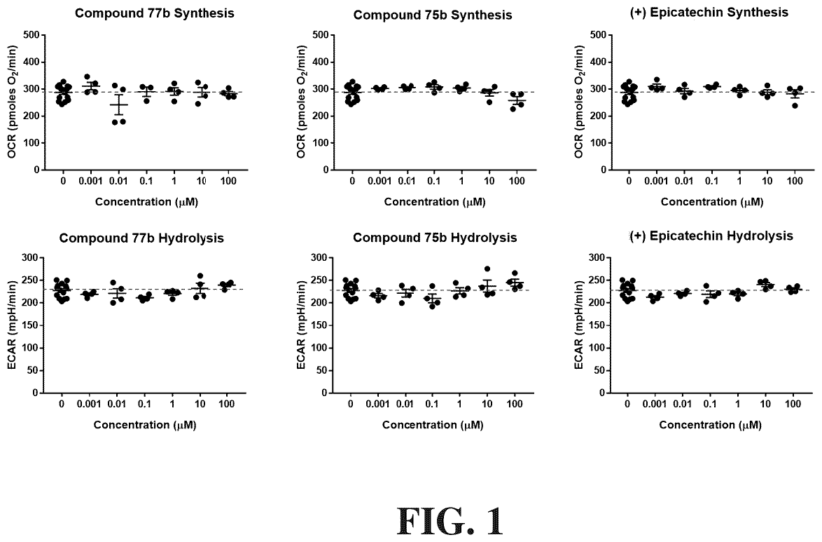

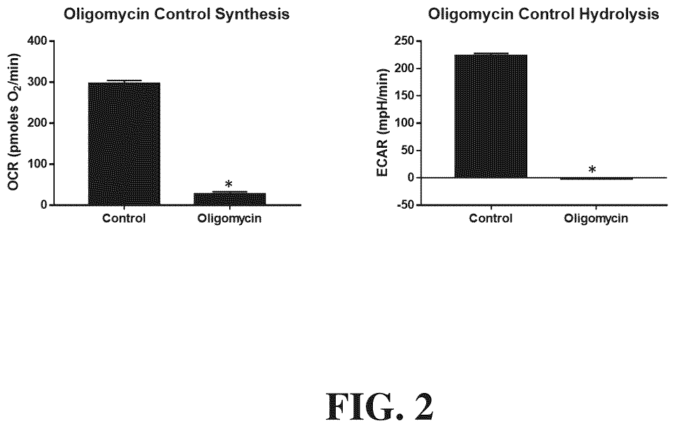

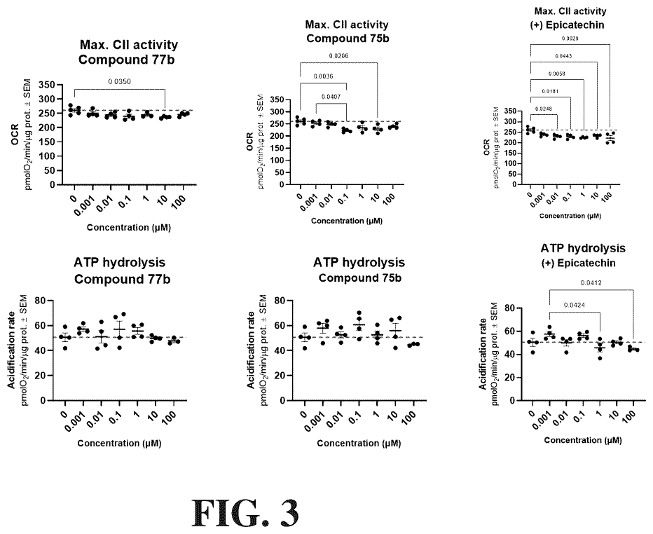

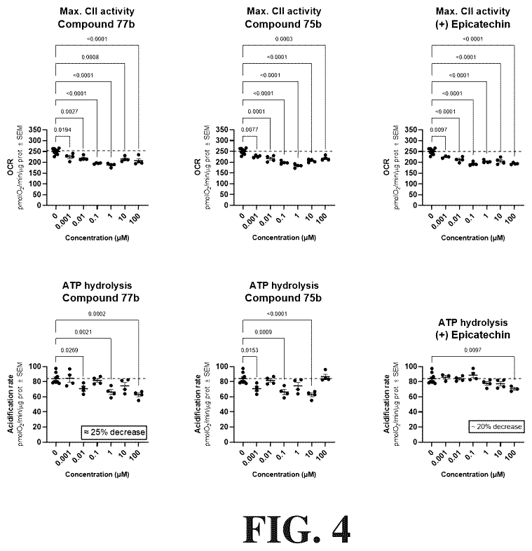

Compound 75b and Compound 77b were tested. There was no change in ATP synthesis for Compound 77b (FIG. 1). There was also no change in ATP synthesis for epicatechin. Oligomycin controls worked as expected for both ATP synthesis and hydrolysis (FIG. 2), but only very mild changes in hydrolysis were observed for Compounds 75b and 77b in this assay. Therefore, frozen mitochondria were used to robustly test hydrolysis. Epicatechin showed no synthesis change with Complex I or Complex II substrates and showed a 10-25% decrease in hydrolysis. In fresh mitochondria, Compound 77b showed no change in synthesis and a 20% decrease in hydrolysis with Complex II, and no change in synthesis and no clear change in hydrolysis in Complex I. In frozen mitochondria, Compound 77b showed a 25% decrease in hydrolysis (FIGS. 3 and 4).

All documents, including patents, patent application and publications cited herein, including all documents cited therein, tables, and drawings, are hereby expressly incorporated by reference in their entirety for all purposes.

While the foregoing written description of the compounds, uses, and methods described herein enables one of ordinary skill in the art to make and use the compounds, uses, and methods described herein, those of ordinary skill in the art will understand and appreciate the existence of variations, combinations, and equivalents of the specific embodiment, method, and examples herein. The compounds, uses, and methods provided herein should therefore not be limited by the above-described embodiments, methods, or examples, but rather encompasses all embodiments and methods within the scope and spirit of the compounds, uses, and methods provided herein.

1. A compound of Formula (I): 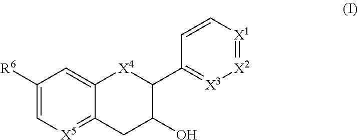
or a pharmaceutically acceptable salt, stereoisomer or tautomer thereof, wherein:
X¹ is N or CR¹;
R¹ is H, OH, halo, or C₁₋₆ alkoxy;
X² is N or CR²;
R² is H, OH, halo, C₁₋₆ alkyl optionally substituted with one or more halo, or C₁₋₆ alkoxy;
X³ is N or CR³;
R³ is H or OH;
X⁴ is O or NR⁴;
R⁴ is H or CH₃;
X⁵ is N or CR⁵;
R⁵ is H, OH, or C₁₋₆ alkoxy;
provided that if X⁴ is NR⁴, none of X¹, X², X³, and X⁵ is N, and if X⁴ is O, then one of X¹, X², X³, and X⁵ is N; and
R⁶ is H, OH, or C₁₋₆ alkoxy.

2. The compound of claim 1, wherein the compound is of Formula (Ia):

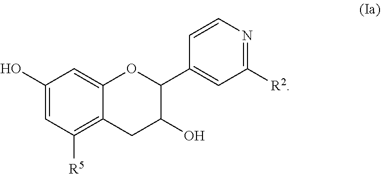

3. The compound of claim 2, wherein R⁵ is H or OH.

4. The compound of claim 2 or 3, wherein R² is H or methoxy.

5. The compound of claim 1, wherein the compound is of Formula (Ib):

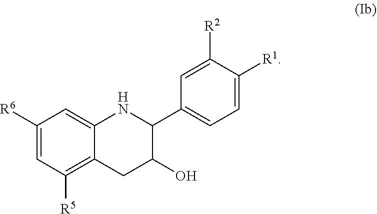

6. The compound of claim 5, wherein R⁵ is H or methoxy.

7. The compound of claim 5 or 6, wherein R⁶ is H or methoxy.

8. The compound of claim 1, wherein the compound is of Formula (Ic):

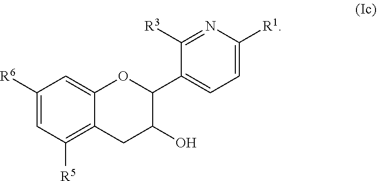

9. The compound of claim 8, wherein R⁵ is H or OH.

10. The compound of claim 8 or 9, wherein R⁶ is H or OH.

11. The compound of any one of claims 8-10, wherein R³ is H or OH.

12. The compound of any one of claims 8-11, wherein R¹ is H or methoxy.

13. The compound of claim 1, wherein the compound is of Formula (Id):

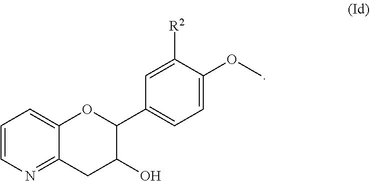

14. The compound of claim 13, wherein R² is F or CF₃.

15. The compound of claim 1, wherein the compound is of Formula (Ie):

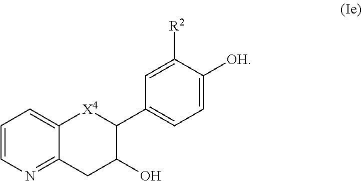

16. The compound of claim 15, wherein R² is H, F, or CF₃.

17. The compound of claim 15, wherein the compound is of Formula (If):

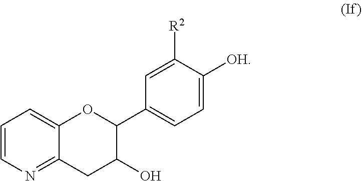

18. The compound of claim 17, wherein R² is H, F, or CF₃.

19. The compound of claim 15, wherein the compound is of Formula (Ig):

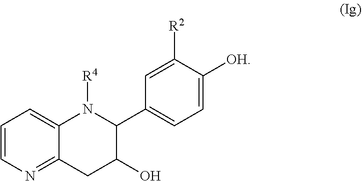

20. The compound of claim 19, wherein R⁴ is methyl.

21. The compound of claim 19 or 20, wherein R² is F or CF₃.

22. The compound of claim 1, wherein the compound is of Formula (Ih):

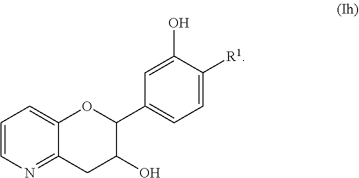

23. The compound of claim 22, wherein R¹ is H or F.

24. The compound of claim 1, wherein the compound is of Formula (Ii):

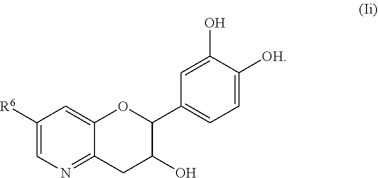

25. The compound of claim 24, wherein R⁶ is H or OH.

26. A compound of Formula (II): 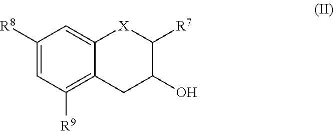
or a pharmaceutically acceptable salt, stereoisomer or tautomer thereof, wherein:
X is O or NCH₃;
R⁷ is C₁₋₆ alkyl, C₃₋₁₀ cycloalkyl optionally substituted with one or more OH or C₁₋₆ alkoxy, or 5-6 membered heterocyclyl optionally substituted with one or more C₁₋₆ alkyl; and
R⁸ and R⁹ are each independently H or OH.

27. The compound of claim 26, wherein the compound is of Formula (IIa):

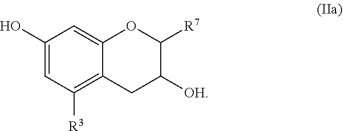

28. The compound of claim 27, wherein the compound is of Formula (IIb):

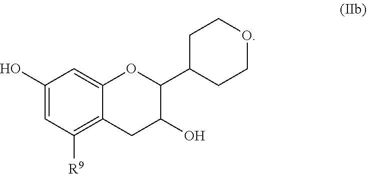

29. The compound of claim 28, wherein R⁹ is H.

30. The compound of claim 28, wherein R⁹ is OH.

31. The compound of claim 26, wherein the compound is of Formula (IIc): 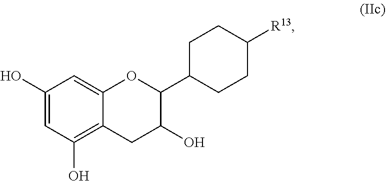
wherein R¹³ is H or OR¹⁴; and
R¹⁴ is H or methyl.

32. The compound of claim 31, wherein R¹³ is H.

33. The compound of claim 31, wherein R¹³ is OR¹⁴, and R¹⁴ is H.

34. The compound of claim 31, wherein R¹³ is OR¹⁴, and R¹⁴ is methyl.

35. A compound of Formula (III): 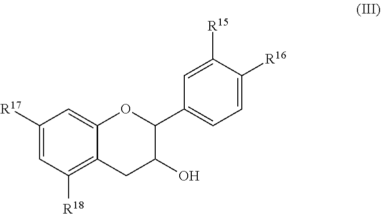
or a pharmaceutically acceptable salt, stereoisomer or tautomer thereof, wherein:
R¹⁵ is H, C₁₋₆ alkoxy, halo, or C₁₋₆ alkyl optionally substituted with halo;
R¹⁶ is H, halo, or OH; and
R¹⁷ and R¹⁸ are each independently H or OH.

36. The compound of claim 35, wherein R¹⁵ is OH.

37. The compound of claim 35, wherein R¹⁵ is methoxy.

38. The compound of claim 35, wherein R¹⁵ is CF₃.

39. The compound of any one of claims 35-38, wherein R¹⁶ is OH.

40. The compound of any one of claims 35-38, wherein R¹⁶ is F.

41. The compound of any one of claims 35-40, wherein R¹⁷ is OH.

42. The compound of any one of claims 35-41, wherein R¹⁸ is H.

43. A compound selected from the group consisting of: 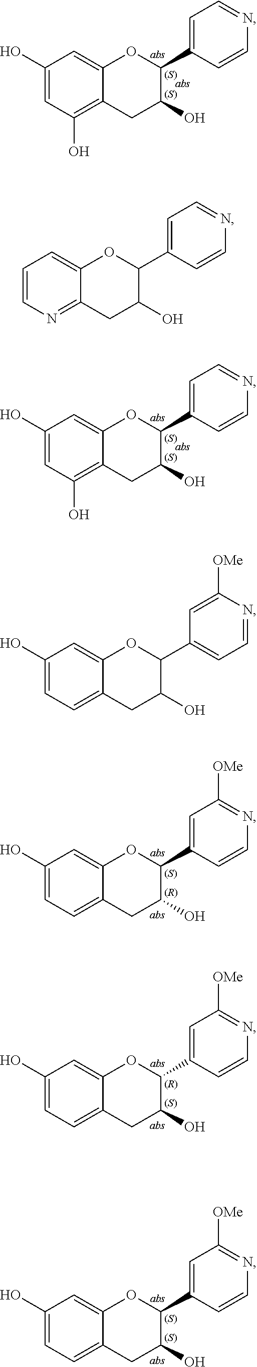 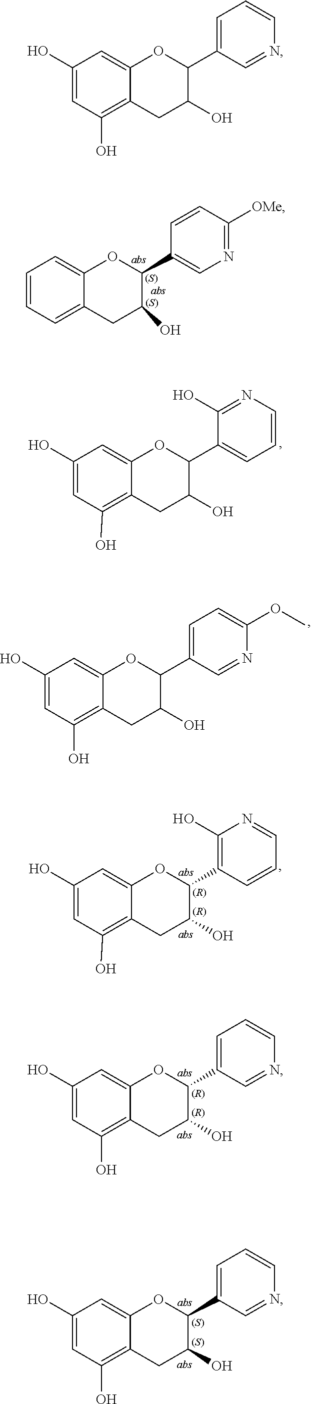 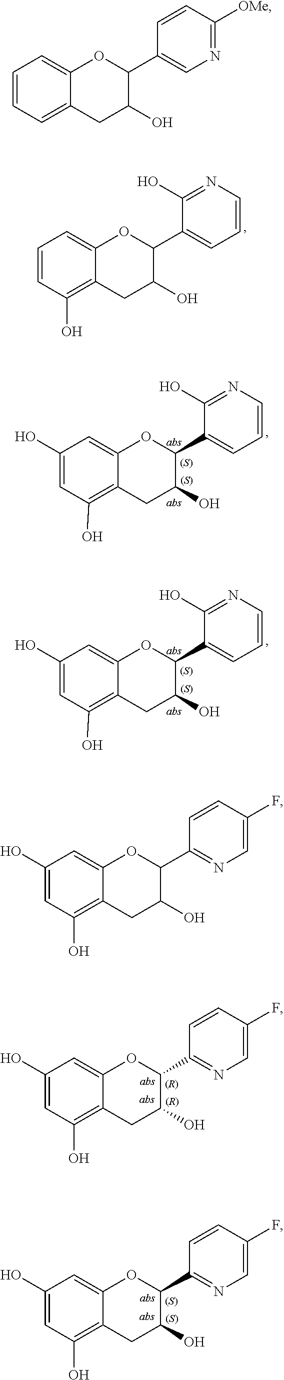 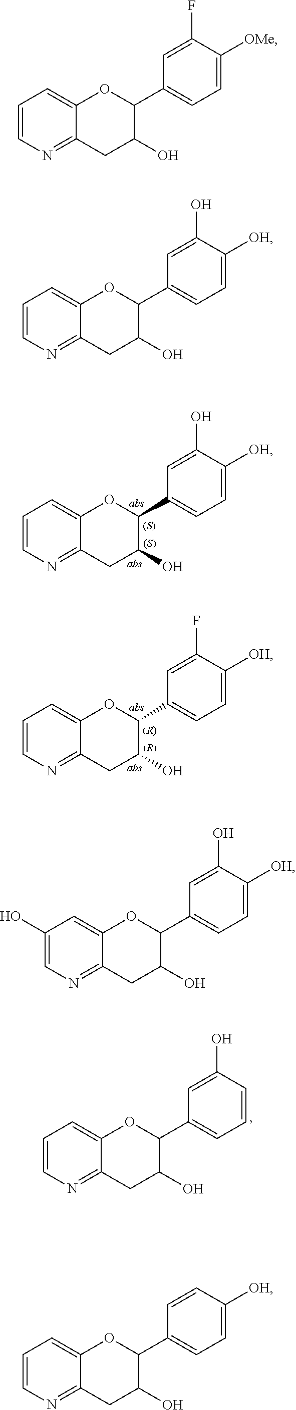 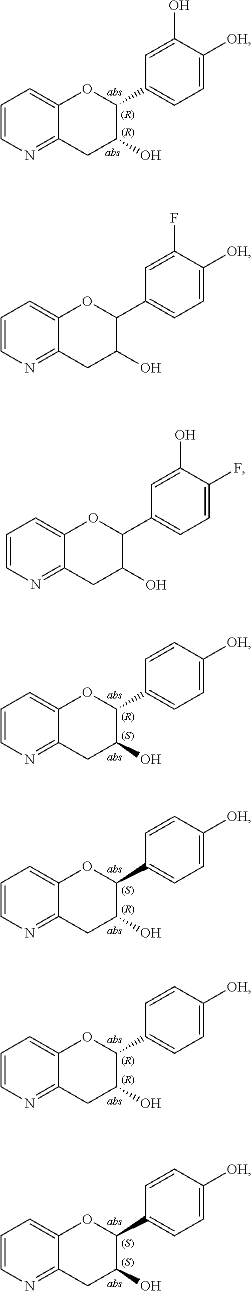 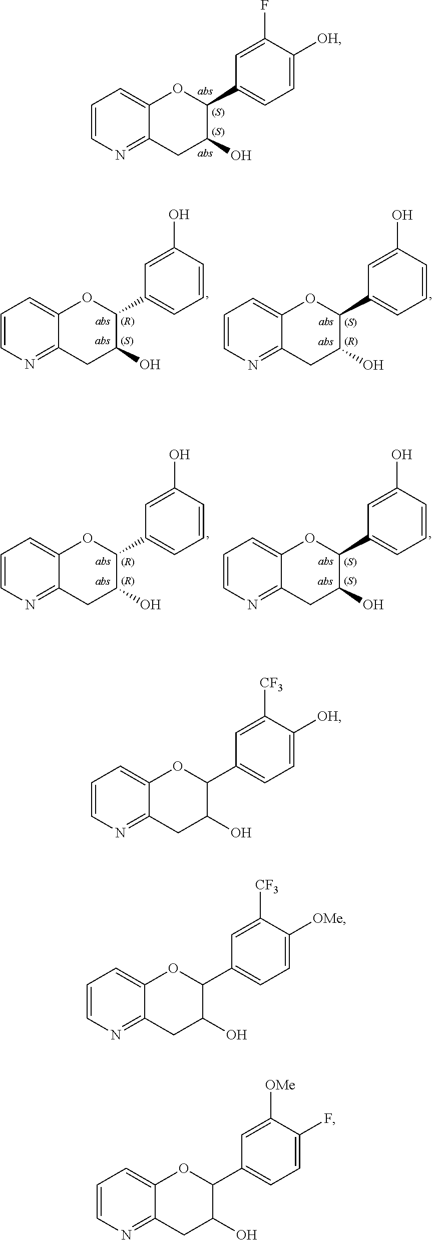 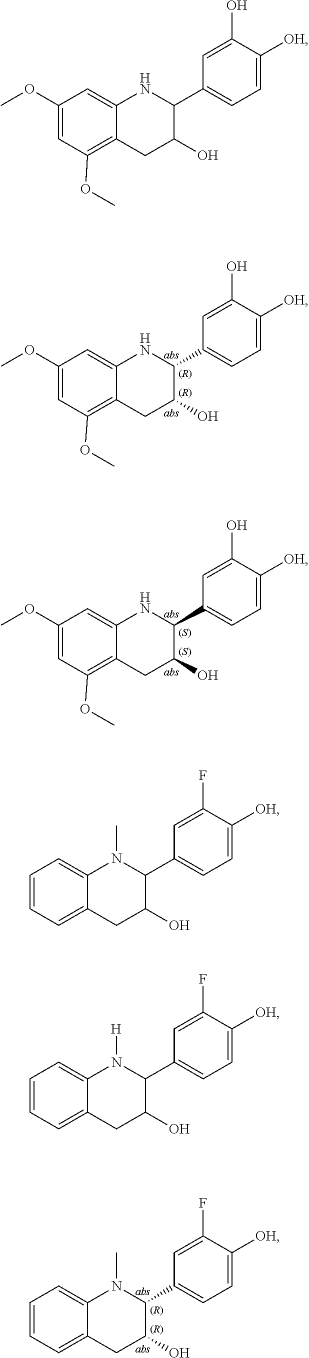 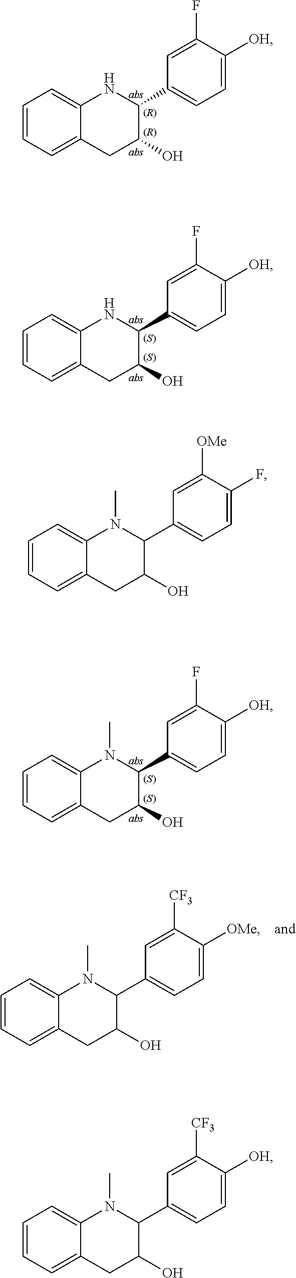
or a pharmaceutically acceptable salt, stereoisomer or tautomer thereof.

44. A compound selected from the group consisting of: 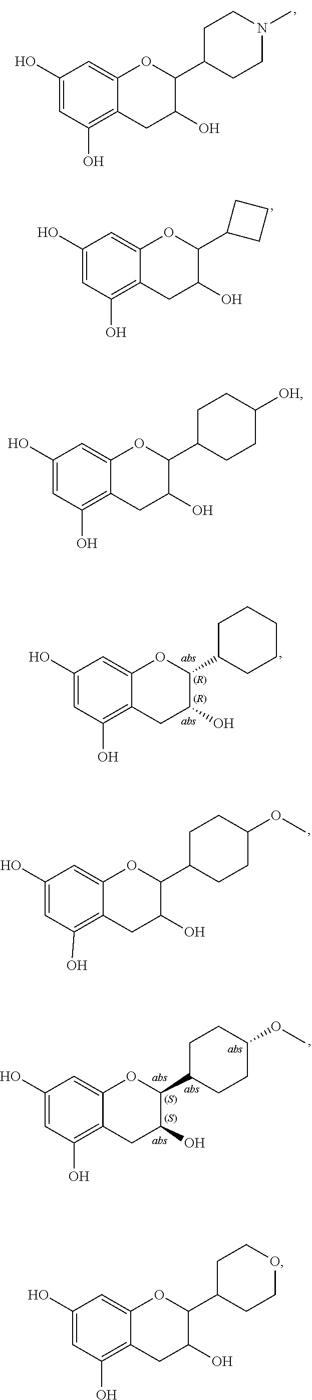 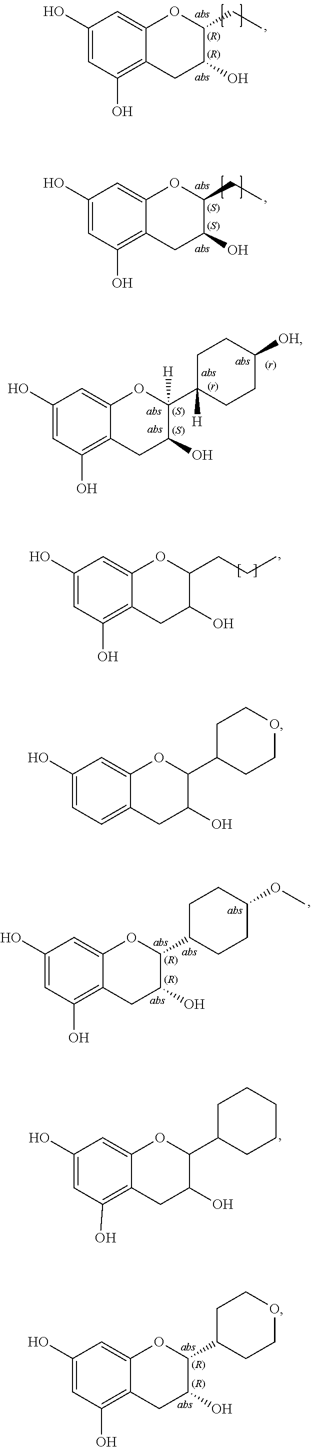 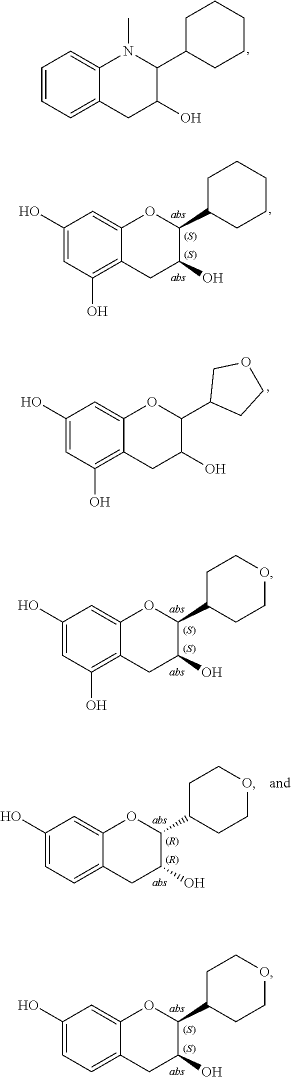
or a pharmaceutically acceptable salt, stereoisomer or tautomer thereof.

45. A compound selected from the group consisting of: 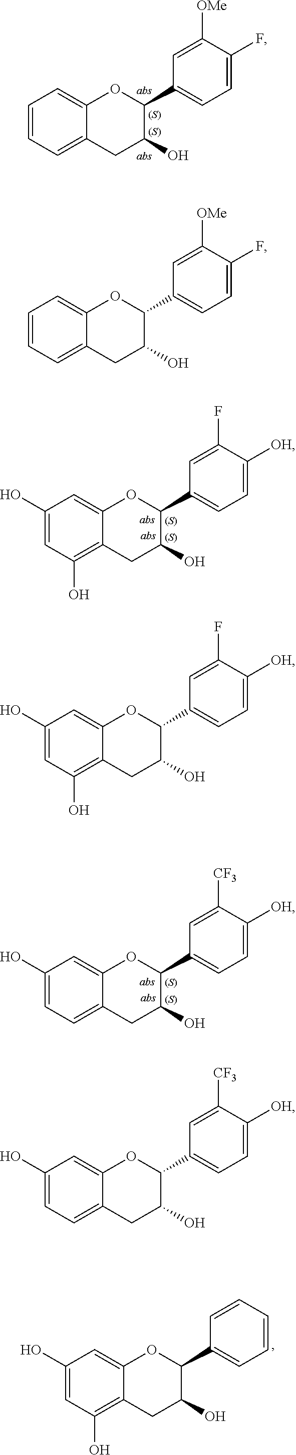 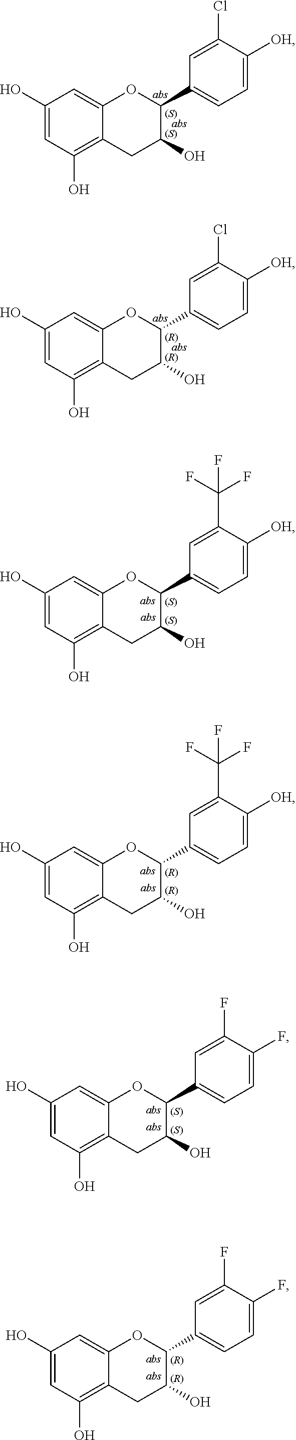 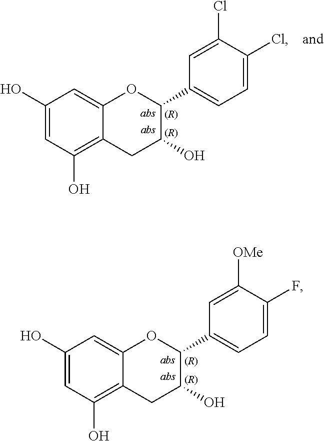
or a pharmaceutically acceptable salt, stereoisomer or tautomer thereof.

46. A compound selected from the group consisting of 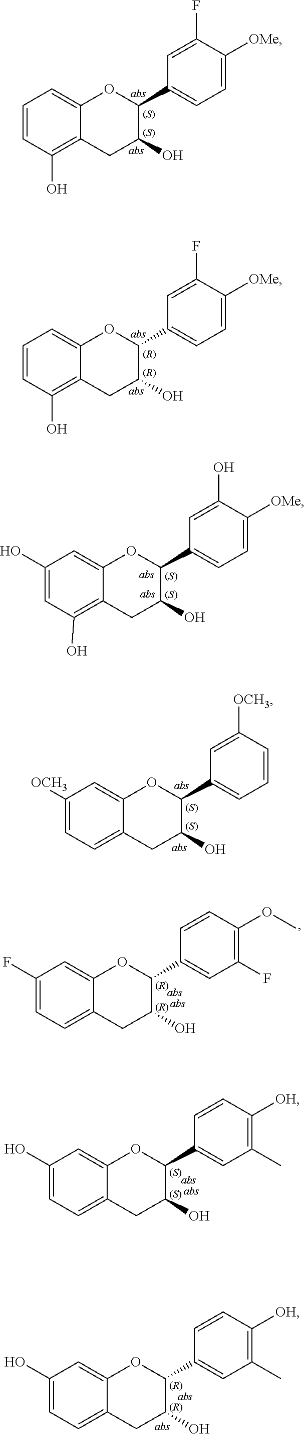 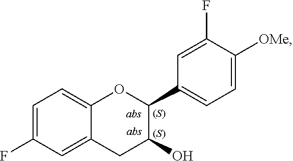 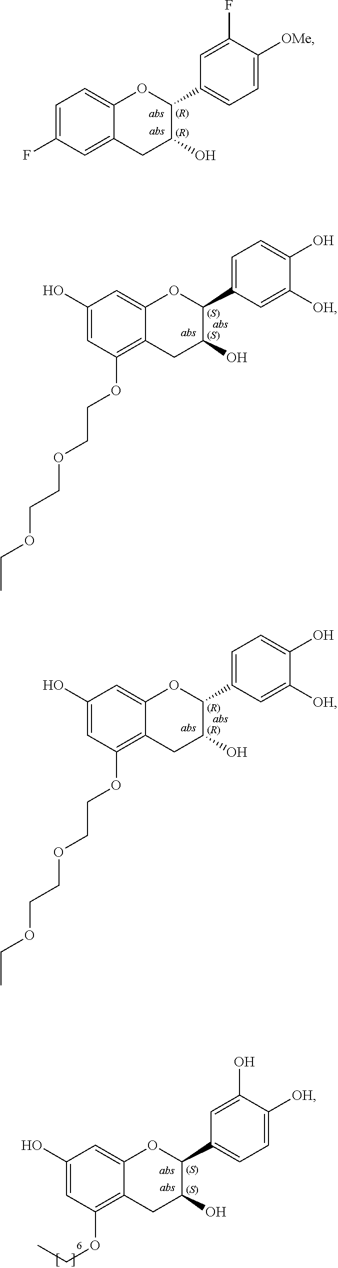 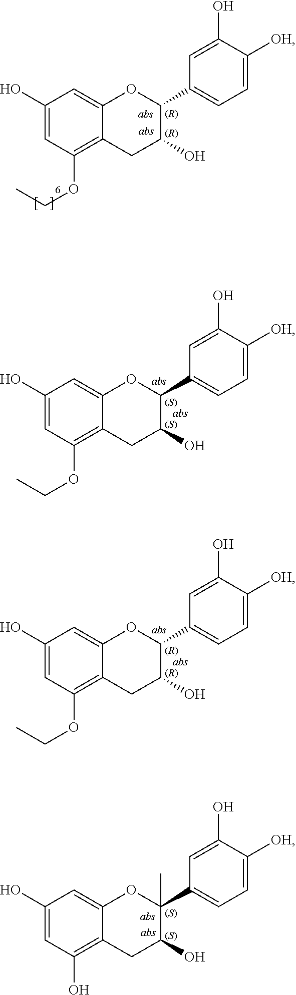 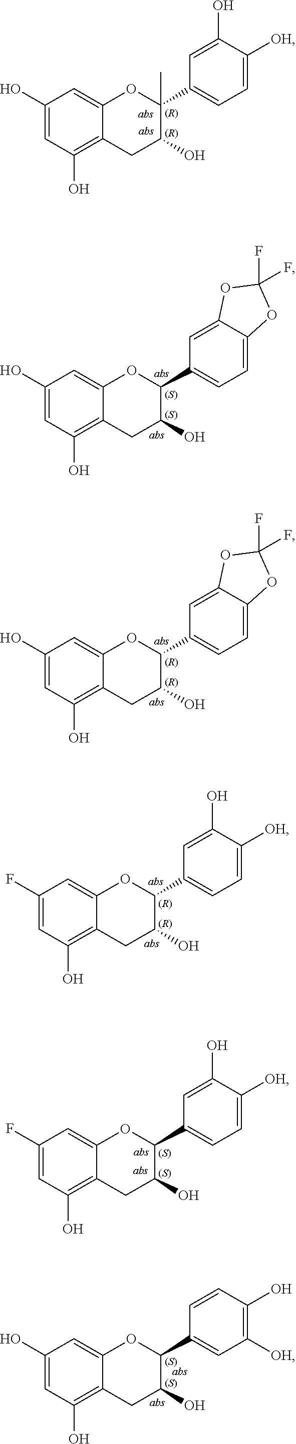 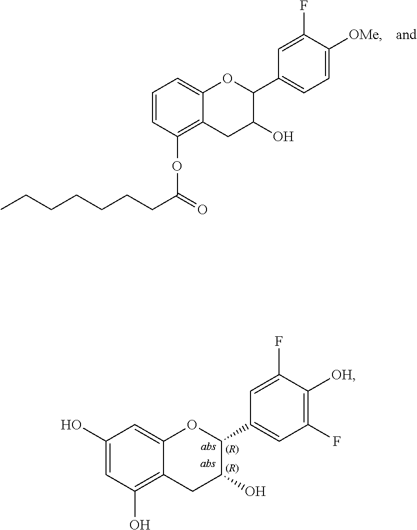
or a pharmaceutically acceptable salt, stereoisomer or tautomer thereof.

47. A pharmaceutical composition comprising a compound of any one of claims 1-46, a pharmaceutically acceptable salt thereof, and a pharmaceutically acceptable carrier.

48. A compound, or a pharmaceutically acceptable salt thereof, of any one of claims 1-46 for use as therapeutically active substance.

49. Use of a compound, or a pharmaceutically acceptable salt thereof, of any one of claims 1-46 or a pharmaceutical composition of claim 46 for the preparation of a medicament for the treatment of a disease, disorder or condition.
